(19) United States
(12) Patent Application Publication
(10) Pub. No.: US 2025/0382627 A1
(43) Pub. Date: Dec. 18, 2025

(54) ENGINEERING REMONTANT FLOWERING IN ROSACEAE

(71) Applicant: J.R. Simplot Company, Boise, ID (US)

(72) Inventors: Michele Krucker, Boise, ID (US); Vanessa Roullier, Boise, ID (US)

(21) Appl. No.: 19/230,543

(22) Filed: Jun. 6, 2025

Related U.S. Application Data
(60) Provisional application No. 63/659,698, filed on Jun. 13, 2024.

(51) Int. Cl. C12N 15/82; C12N 9/22; C12N 15/11

(52) U.S. Cl. CPC C12N 15/827; C12N 9/226; C12N 15/11; C12N 15/8205; C12N 15/8213; C12N 2310/20

(57) ABSTRACT

The present disclosure relates to cultivated Rosaceae plants, such as strawberries, blackberries, and raspberries, having an early flowering trait resulting from genetically engineered Terminal Flowering alleles (TFL1) or homologs thereof that reduce or knockout TFL1 protein function. The disclosure further teaches methods of producing the same.

### CROSS-REFERENCE TO RELATED APPLICATION

This application claims the benefit of priority to U.S. Provisional Application No. 63/659,698, filed Jun. 13, 2024, the entire contents of which is incorporated herein by reference.

### FIELD

The field of the invention concerns genetically modified Rosaceae plants having reduced activity of a Terminal Flower (TFL) gene, resulting in earlier flowering and higher yield.

### REFERENCE TO AN ELECTRONIC SEQUENCE LISTING

The contents of the electronic sequence listing (JRSI_090_00US_SeqList_ST26.xml; Size: 157,097 bytes; and Date of Creation: May 13, 2024) are herein incorporated by reference in its entirety.

### BACKGROUND

Flowering in plants is determined by the timing of the transition from vegetative to reproductive stage. Endogenous signal transduction cascade initiated or altered by environmental factors causes this phase change during the life cycle of a plant. A set of genes involved in signal cascades finely regulate the transition to flowering. Mechanisms to reduce the time to flower and increase yield are needed.

### SUMMARY

In some aspects, the disclosure relates to a cultivated Rosaceae plant, plant part, or plant cell having genetically engineered Terminal Flowering d1 and d2 alleles (TFL1d1 and TFL1d2), or homologs thereof, wherein each TFL1d allele has one or more edits that reduce or knockout protein function. In some aspects, the plant, plant part, or plant cell further includes one or more edits in a TFL1a, TFL1b1, TFL1b2, TFL1c, or TFL1e allele, or homologs thereof, that reduce or knockout protein function.

In some aspects, the techniques described herein relate to a cultivated Rosaceae plant, wherein the plant flowers earlier than a cultivated Rosaceae plant of the same variety having wildtype TFL1d1 and TFL1d2 alleles when grown under the same environmental conditions. In some aspects, the plant flowers at least one week earlier than a cultivated Rosaceae plant of the same variety having wildtype TFL1d1 and TFL1d2 alleles when grown under the same environmental conditions. In some aspects, the plant flowers between two and 12 weeks earlier than a cultivated Rosaceae plant of the same variety having wildtype TFL1d1 and TFL1d2 alleles when grown under the same environmental conditions. In some aspects, the plant flowers between 12 and 20 weeks earlier than a cultivated Rosaceae plant of the same variety having wildtype TFL1d1 and TFL1d2 alleles when grown under the same environmental conditions.

In some aspects, the disclosure relates to a cultivated Rosaceae plant, wherein the plant has increased yield compared to a cultivated Rosaceae plant of the same variety having wildtype TFL1d1 and TFL1d2 alleles when grown under the same environmental conditions. In some aspects, the plant has between 1% and 10% increase in yield compared to a cultivated Rosaceae plant of the same variety having wildtype TFL1d1 and TFL1d2 alleles when grown under the same environmental conditions. In some aspects, the plant has between 10% and 25% increase in yield compared to a cultivated Rosaceae plant of the same variety having wildtype TFL1d1 and TFL1d2 alleles when grown under the same environmental conditions. In some aspects, the plant has between 25% and 50% increase in yield compared to a cultivated Rosaceae plant of the same variety having wildtype TFL1d1 and TFL1d2 alleles when grown under the same environmental conditions. In some aspects, the plant has between 50% and 100% increase in yield compared to a cultivated Rosaceae plant of the same variety having wildtype TFL1d1 and TFL1d2 alleles when grown under the same environmental conditions.

In some aspects, the techniques described herein relate to a cultivated Rosaceae plant, plant part, or plant cell, wherein the plant, plant part, or plant cell is a species of Fragaria.

In some aspects, the disclosure relates to a cultivated Fragaria sp. plant, plant part, or plant cell having an early flowering trait, wherein said early flowering trait is caused by genetically engineered Terminal Flowering d1 and d2 alleles (TFL1d1 and TFL1d2), and wherein each TFL1d allele has one or more edits that reduce or knockout protein function. In some aspects, each TFL1d allele has one or more edits in exon 2. In some aspects, each TFL1d allele has one or more edits that disrupt the TFL protein interaction with a 14-3-3 protein. In some aspects, each TFL1d allele has one or more edits that disrupt the TFL proteins substrate binding. In some aspects, the plant further includes one or more edits in a TFL1a, TFL1b1, TFL1b2, TFL1c, and/or TFL1e allele that reduce or knockout protein function. In some aspects, the plant has increased yield compared to another cultivated Fragaria sp. plant of the same variety having wildtype TFL1d1 and TFL1d2 alleles when grown under the same environmental conditions.

In some aspects, the techniques described herein relate to a method for producing a cultivated Rosaceae plant having increased yield, the method including: targeting one or more TFL1 alleles in a Rosaceae plant, plant part, or plant cell to reduce or knockout TFL1 function, wherein at least one of the targeted TFL1 alleles shares 80% or more sequence identity with SEQ ID NO: 62, and producing a cultivated Rosaceae plant therefrom, wherein the plant has increased yield compared to another cultivated Rosaceae plant of the same variety having wild-type TFL1 alleles and grown under the same conditions.

In some aspects, the techniques described herein relate to a method for producing a cultivated Rosaceae plant, plant part, or plant cell having an early flowering trait, the method including: targeting one or more TFL1 alleles in a Rosaceae plant, plant part, or plant cell to reduce or knockout TFL1 function, wherein at least one of the targeted TFL1 alleles shares 80% or more sequence identity with SEQ ID NO: 62, and producing a cultivated Rosaceae plant therefrom, wherein the plant flowers earlier compared to another cultivated Rosaceae plant of the same variety having wild-type TFL1 alleles and grown under the same conditions.

### BRIEF DESCRIPTION OF THE DRAWINGS

FIG. 1 is a graphic of various member of the PEBP Superfamily generated with InterPro (available on the world wide web at ebi.ac.uk/interpro/). The vertical rectangle shows an exemplary region targeted for genomic editing in the present disclosure.

FIG. 2 is a partial alignment of various strawberry TFL and FT-like genes generated with QIAGEN® CLC Genomics. The top two stars indicate key amino acids that differentiate TFL from FT. FT will have a Tyr-85 (Y) and Gln-140 (Q) while TFL will have a His-88 (H) and Asp-144 (D). The bottom two stars also indicate FTs with a Try-134 (Y) and Trp-138 (W).

FIG. 3A is a partial protein alignment of TFL genes in other Rosaceae species generated with QIAGEN® CLC Genomics.

FIG. 3B is a phylogenetic tree of the TFL homologs shown in FIG. 3A generated with QIAGEN® CLC Genomics.

FIG. 4 is a schematic representation of the TFL1 gene generated with QIAGEN® CLC Genomics. The arrow labeled TFLg6 is an example region in exon 2 targeted for editing.

FIG. 5 is a line graph plotting the weeks to flowering after planting in relation to the number of TFL alleles edited.

FIG. 6 is a bar graph showing the percentage of plants to reach anthesis by 9 weeks after planting for individual plant events, grown in Salinas, California in 2022-2023. Numbers shown in parentheticals for each event represent the number of TFL1 alleles edited. Striped bars and plant events scoring 0% did not have both TFL1d1 and TFL1d2 alleles edited.

FIG. 7 is a bar graph showing the percentage of plants to reach anthesis by 7 weeks after planting for individual plant events, grown in Oxnard, California in 2022-2023. Numbers shown in parentheticals for each event represent the number of TFL1 alleles edited. Striped bars and plant events scoring 0% did not have both TFL1d1 and TFL1d2 alleles edited.

FIG. 8 is a bar graph showing the percentage of plants to reach anthesis by 7 weeks after planting for individual plant events, grown in Oxnard, California in 2022-2023. Striped bars and plant events scoring 0% did not have both TFL1d1 and TFL1d2 alleles edited.

FIG. 9 is a bar graph showing the percentage of plants to reach anthesis by 8 weeks after planting for individual plant events, grown in Oxnard, California in 2023. Numbers shown in parentheticals for each event represent the number of TFL1 alleles edited. Striped bars and plant events scoring 0% did not have both TFL1d1 and TFL1d2 alleles edited.

FIG. 10 is a bar graph showing the percentage of plants to reach anthesis by 8 weeks after planting for individual plant events, grown in Oxnard, California in 2023-2024. Numbers shown in parentheticals for each event represent the number of TFL1 alleles edited. Striped bars and plant events scoring 0% did not have both TFL1d1 and TFL1d2 alleles edited.

FIG. 11 is a bar graph showing the percentage of plants to reach anthesis by 8 weeks after planting for individual plant events, grown in Salinas, California in 2023-2024. Numbers shown in parentheticals for each event represent the number of TFL1 alleles edited. Striped bars and plant events scoring 0% did not have both TFL1d1 and TFL1d2 alleles edited

FIG. 12A is an amino acid alignment of allele TFL1d2 in line S4A-018 (SEQ ID NO: 61) compared to wildtype (SEQ ID NO: 62), showing a deletion of V73, prepared using BLAST®.

FIG. 12B is an amino acid alignment of allele TFL1d2 in line S4A-162 (SEQ ID NO: 47) compared to wildtype (SEQ ID NO: 62), showing a D72E change and deletion of V73, prepared using BLAST®.

FIG. 12C is an amino acid alignment of allele TFL1d2 in line S4A-019 (SEQ ID NO: 43) compared to wildtype (SEQ ID NO: 62), showing an amino acid change from V73-G75 with an early stop codon, prepared using BLAST®.

FIG. 12D is an amino acid alignment of allele TFL1d1 in line S4A-176 (SEQ ID NO: 71) compared to wildtype (SEQ ID NO: 62), showing a deletion spanning V73-D78, prepared using BLAST®.

### DETAILED DESCRIPTION

In the description, which follows, a number of terms are used. In order to provide a clear and consistent understanding of the specification and claims, including the scope to be given such terms, the following definitions are provided:

“Bare root” refers to the technique wherein plants are removed from the soil (this may also be referred to as harvested) when they are dormant, and the soil is removed from their roots. Bare root plants may then be stored and re-planted while still dormant. Examples of plants that may be sold or transplanted as bare root plants include fruit trees, strawberries, raspberries, roses, and ornamental trees and shrubs.

“Commercial production field” or “fruiting field” refers to a field or environment where strawberry plants are grown for fruit production.

“Conditioned” or “conditioning” refers to the process of growing strawberry plug plants such that the plants undergo vernalization.

As used herein, the term “independently of vernalization” or “independently of temperature and/or photoperiod” refers to plants which did not experience conditioning, or did not experience sufficient vernalization, for example where the plant may have been subjected to low temperature briefly, but does not receive enough Accumulative Chilling Unit (e.g., the plant receives an ACU less than 70° C. hr, less than 100° C. hr, or less than 200° C. hr.), or the situation where the plant may have been subjected to photoperiod conditions briefly, but the duration is so short that it does not materially change the flowering time of the plants.

“Cross”, “crossing”, “cross pollination” or “cross-breeding” refer to the process by which the pollen of one flower on one plant is applied (artificially or naturally) to the ovule (stigma) of a flower on another plant.

“Day neutral” refers to a plant that produces flowers regardless of the length of the period of light exposure. A day neutral variety is sometimes referred to as a perpetual flowering variety, or a recurrent variety, or a remontant variety (repeat flowering), or an ever-bearing variety, or a long-day variety.

“Everbearing” refers to a strawberry variety that produces two or three harvests of strawberry fruit per year, one in the spring and another in the late summer or fall, and under ideal conditions, a third harvest.

As used herein, the terms “endogenous,” and “native” refer to the naturally occurring copy of a gene or promoter.

“Foreign,” or “exogenous” with respect to a nucleic acid, means that that nucleic acid is derived from non-plant organisms, or derived from a plant that is not the same species as the plant to be transformed, or is derived from a plant that cannot be crossed with the plant to be transformed.

“Genome” refers to the complete DNA component of an organism. In plants, a genome may be a nuclear genome, a chloroplast genome, or a mitochondrial genome.

“Genetically modified” refers to a man-made change in a genome of an organism. The genetic modification may be induced by a mutagen, or generated by targeted genome editing.

“High elevation” refers to an elevation within a range of about 3000 to 6000 feet above sea level.

“Homologous” or “homologue” or “ortholog” is known in the art and refers to related sequences that share a common ancestor or family member and are determined based on the degree of sequence identity. They refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant disclosure such as deletion or insertion of one or more nucleotides that do not substantially alter the functional properties of the resulting nucleic acid fragment relative to the initial, unmodified fragment. It is therefore understood, as those skilled in the art will appreciate, that the disclosure encompasses more than the specific exemplary sequences. These terms describe the relationship between a gene found in one species, subspecies, variety, cultivar or strain and the corresponding or equivalent gene in another species, subspecies, variety, cultivar or strain. For purposes of this disclosure, homologous sequences are compared. “Homologous sequences” or “homologues” or “orthologs” are thought, believed, or known to be functionally related. A functional relationship may be indicated in any one of a number of ways, including, but not limited to: (a) degree of sequence identity and/or (b) the same or similar biological function. Where a particular sequence is said to have a specific percent identity to a reference sequence of a defined length, the percent identity is relative to the reference sequence. Thus, a sequence that is 50% identical to a reference sequence that is 100 amino acids (or 100 nucleotides long) can be a 50 amino acid polypeptide or a 50 nucleotide sequence that is completely identical to a 50 amino acid long portion of the reference polypeptide or a 50 nucleotides long portion of the reference nucleotide sequence. It might also be a 100 amino acid long polypeptide, or a 100 nucleotide sequence, which is 50% identical to the reference polypeptide or the reference nucleotide sequence over its entire length. Of course, other sequences unspecified may also meet the same criteria. Homology can be determined using software programs readily available in the art, such as NCBI BLAST (Basic Local Alignment Search Tool), using default parameters.

“June-Bearing” refers to a strawberry variety that produces fruit around the month of June. The June-bearing strawberry varieties can be divided into ‘early season’, ‘early midseason’, ‘midseason’, ‘late midseason’, and ‘late season’ referring to the relative timing of when fruiting begins. For example, relative to the early season varieties, fruiting begins about 5 days later for the early midseason variety; fruiting begins about 8 days later for the midseason varieties; fruiting begins about 10 days later for the late midseason varieties; and fruiting begins about 14 days later for the late season varieties. June-bearing varieties may also be referred to as a short-day variety or a seasonal flowering variety.

“Locus”. A locus confers one or more traits and may comprise one or more genes.

“Long day” is a 24 hour period (a day) with more than 12 hours of light.

“Low elevation” refers to an elevation of less than sea level to about 3000 feet above sea level.

“Non-natural mutant” refers to mutants or genetic changes induced or created by humans.

As used herein, the term “transgenic” refers to an organism that comprises genetic material from another species has been artificially introduced. The term “non-transgenic” thus refers to an organism which does not comprise genetic material from another species introduced by artificial (non-breeding) means.

“Offspring” refers to any plant progeny derived from an initial variety (parent plant). For instance, an offspring plant may be obtained by cloning (asexual reproduction) or selfing of a parent plant or by crossing two parental plants and include selfings as well as the F1 or F2 or still further generations.

“Photoperiod” refers to the length of time in a 24-hour cycle that a plant receives illumination. In some embodiments, a photoperiod is a short day with less than 12 hours of illumination per 24-hour period. In some embodiments, a photoperiod is a long day with more than 12 hours of illumination per 24-hour period.

“Plant part” refers to any part of a plant including but not limited to a plant cell, embryo, shoot, root, stem, seed, stipule, leaf, petiole, petal, calyx, sepal, flower, ovule, bract, branch, internode, pubescence, tiller, rhizome, frond, blade, ovule, pollen, stamen, runner, stolon, achene.

“Plug plants” are young plants grown with the intent of being replanted in a secondary location. Plug plants have a characteristic root ball that improves the chances for survival after transplanting, and increases the growth rate after transplanting into the fruit production field. Plug plants are also referred to as a daughter plants.

“Promoter” refers to a DNA sequence capable of controlling the expression of a coding sequence or functional RNA. The promoter sequence consists of proximal and more distal upstream elements, the latter elements often referred to as enhancers. Accordingly, an “enhancer” is a DNA sequence that can stimulate promoter activity, and may be an innate element of the promoter or a heterologous element inserted to enhance the level or tissue specificity of a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity.

A “plant promoter” is a promoter capable of initiating transcription in plant cells whether or not its origin is a plant cell, for example, it is well known that Agrobacterium promoters are functional in plant cells. Thus, plant promoters include promoter DNA obtained from plants, plant viruses and bacteria such as Agrobacterium and Bradyrhizobium bacteria. A plant promoter can be a constitutive promoter or a non-constitutive promoter.

A “constitutive promoter” is a promoter which is active under most conditions and/or during most development stages. There are several advantages to using constitutive promoters in expression vectors used in plant biotechnology, such as: high level of production of proteins used to select transgenic cells or plants; high level of expression of reporter proteins or scorable markers, allowing easy detection and quantification; high level of production of a transcription factor that is part of a regulatory transcription system; production of compounds that requires ubiquitous activity in the plant; and production of compounds that are required during all stages of plant development. Non-limiting exemplary constitutive promoters include: CaMV 35S promoter, opine promoter, ubiquitin promoter, and alcohol dehydrogenase promoter.

A “non-constitutive promoter” is a promoter which is active under certain conditions, in certain types of cells, and/or during certain development stages. For example, tissue specific, tissue preferred, cell type specific, cell type preferred, inducible promoters, and promoters under development control are non-constitutive promoters. Examples of promoters under developmental control include promoters that preferentially initiate transcription in certain tissues, such as stems, leaves, roots, or seeds.

An “inducible” or “repressible” promoter is a promoter which is under chemical or environmental factors control. Examples of environmental conditions that may affect transcription by inducible promoters include anaerobic conditions, or certain chemicals, or the presence of light.

A “tissue specific” promoter is a promoter that initiates transcription only in certain tissues. Unlike constitutive expression of genes, tissue-specific expression is the result of several interacting levels of gene regulation. As such, in the art sometimes it is preferable to use promoters from homologous or closely related plant species to achieve efficient and reliable expression of transgenes in particular tissues. This is one of the main reasons for the large amount of tissue-specific promoters isolated from particular plants and tissues found in both scientific and patent literature.

A “target nucleic acid” as used herein is a polynucleotide (e.g., RNA, DNA) that includes a “target site” or “target sequence.” The terms “target site” or “target sequence” are used interchangeably herein to refer to a nucleic acid sequence present in a target nucleic acid to which a targeting segment of a subject guide nucleic acid will bind, provided sufficient conditions for binding exist. Suitable hybridization conditions include physiological conditions normally present in a cell. For a double stranded target nucleic acid, the strand of the target nucleic acid that is complementary to and hybridizes with the guide nucleic acid is referred to as the “complementary strand”; while the strand of the target nucleic acid that is complementary to the “complementary strand” (and is therefore not complementary to the guide nucleic acid) is referred to as the “noncomplementary strand” or “non-complementary strand”. In embodiments where the target nucleic acid is a single stranded target nucleic acid (e.g., single stranded DNA (ssDNA), single stranded RNA (ssRNA)), the guide nucleic acid is complementary to and hybridizes with single stranded target nucleic acid.

A nucleic acid molecule that binds to an RNA-guided endonuclease (e.g., the Cas9 Polypeptide) and targets the polypeptide to a specific location within the target nucleic acid is referred to herein as a “guide nucleic acid”. When the guide nucleic acid is an RNA molecule, it can be referred to as a “guide RNA” or a “gRNA”. A guide nucleic acid comprises two segments, a first segment (referred to herein as a “targeting segment”); and a second segment (referred to herein as a “protein-binding segment”). By “segment” it is meant a segment/section/region of a molecule, e.g., a contiguous stretch of nucleotides in a nucleic acid molecule. A segment can also mean a region/section of a complex such that a segment may comprise regions of more than one molecule. For example, in some embodiments the protein-binding segment (described below) of a guide nucleic acid is one nucleic acid molecule (e.g., one RNA molecule) and the protein-binding segment therefore comprises a region of that one molecule. In other embodiments, the protein-binding segment (described below) of a guide nucleic acid comprises two separate molecules that are hybridized along a region of complementarity.

The first segment (targeting segment) of a guide nucleic acid (e.g., guide RNA or gRNA) comprises a nucleotide sequence that is complementary to a specific sequence (a target site) within a target nucleic acid (e.g., a target ssRNA, a target ssDNA, the complementary strand of a double stranded target DNA, etc.). The protein-binding segment (or “protein-binding sequence”) interacts with an RNA-guided endonuclease (e.g., Cas9) polypeptide. Site-specific binding and/or cleavage of the target nucleic acid can occur at locations determined by base-pairing complementarity between the guide nucleic acid (e.g., guide RNA) and the target nucleic acid.

The protein-binding segment of a subject guide nucleic acid comprises two complementary stretches of nucleotides that hybridize to one another to form a double stranded RNA duplex (dsRNA duplex).

A subject guide nucleic acid (e.g., guide RNA) linked to a donor polynucleotide forms a complex with a subject RNA-guided endonuclease (e.g., Cas9) (i.e., binds via non-covalent interactions). The guide nucleic acid (e.g., guide RNA) provides target specificity to the complex by comprising a nucleotide sequence that is complementary to a sequence of a target nucleic acid. Thus, the RNA-guided endonuclease (e.g., Cas9) of the complex provides site-specific or “targeted” activity by virtue of its association with the protein-binding segment of the guide nucleic acid.

The term “guide nucleic acid” is inclusive, referring to both dual guide nucleic acids and to single guide nucleic acids and the term “guide RNA” is also inclusive, referring to both dual guide RNA (dgRNA) and single guide RNA (sgRNA).

The term “protospacer” refers to the DNA sequence targeted by a crRNA guide strand.

The “protospacer-adjacent motif” or “PAM” sequence is a 2-6 base pair DNA sequence immediately following the DNA sequence targeted by an RNA-guided endonuclease (e.g., Cas9). The PAM sequences is required for cleavage of the target nucleic acid and varies depending on the source of the RNA-guided endonuclease (e.g., Cas9). For example, in case of the Streptococcus pyogenes Cas9 the PAM sequence is NGG.

“Synthetic promoter” refers to a promoter that is not naturally found in nature. The nucleotide sequence is artificial or synthetic. A synthetic promoter may be a constitutive promoter, it may be a non-constitutive promoter, it may an inducible promoter, or it may be a tissue specific promoter. Exemplary synthetic promoters useful for transgene expression are disclosed in U.S. Pat. No. 9,670,497, which is herein incorporated by reference in its entirety.

Recombinant: the disclosure also provides chimeric or recombinant molecules for altering gene function in a plant. As used herein, the term “chimeric” or “recombinant” when describing a nucleic acid sequence or a protein sequence refers to a nucleic acid or a protein sequence that links at least two heterologous polynucleotides or two heterologous polypeptides into a single macromolecule, or that re-arranges one or more elements of at least one natural nucleic acid or protein sequence. For example, the term “recombinant” can refer to an artificial combination of two otherwise separated segments of sequence, for example, by chemical synthesis or by the manipulation of isolated segments of nucleic acids by genetic engineering techniques.

“Repress” or “repression” refers to any mean that can reduce the activity of a target gene when compared to the activity of a check gene (for example, a wild type allele in the same plant species). The reduction can be at gene expression level, RNA activity level, and/or protein activity level, including but not limited to, reduced gene copy number, reduced gene amplification, reduced mRNA abundance, synthesis rate, and/or stability, reduced protein synthesis, protein abundance, stability, enzymatic activity, or phosphorylation. In some embodiments, the repression happens when a DNA-binding repressor blocks the attachment of RNA polymerase to the promoter, thus preventing transcription of the genes into messenger RNA. In some embodiments, the repression happens when one or more mutations are introduced into the promoter/coding/intron or terminator region of the target gene. In some embodiments, the repression happens when interference RNA is introduced into the plant to inhibit the target gene. The definition also encompasses varies degrees of modified gene activity, such as modified gene activity achieved by gene silencing, loss-of-function mutant, knock-out, knock-down, leaky mutation. The degree to which the function of a target gene is lost can vary. For example, the target gene can completely lose its function (for example, a null mutation), or partially maintain its function, but not at the level of a wild-type check allele (for example, a leaky mutation). In the present disclosure, for instance, repression of a TFL gene, such as TFL1, using known techniques results in reduced TFL1 activity.

“Gene activity” refers to gene expression level, RNA activity level, or protein activity level. As used herein, the term “RNA activity level refers to mRNA abundance, synthesis rate, and/or stability. As used herein, the term “protein activity level” refers to protein abundance, synthesis rate, stability, enzymatic activity, phosphorylation rate.

“Root ball” is a spherically shaped mass of a plant's root system. For the present disclosure strawberry plug plants are grown in such a way to produce a desirable root ball for the purpose of increasing the survival and health of the plant after replanting.

“Season” refers to the time of the year in which a plant is actively growing in size, undergoing phenotypic changes, and is therefore not dormant.

“Sequence identity” or “identity” in the context of two nucleic acid or polypeptide sequences includes reference to the number of residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988). The comparison of sequences and determination of percent identity between two sequences can be accomplished using a mathematical algorithm. An example of a local alignment algorithm utilized for the comparison of sequences is the NCBI Basic Local Alignment Search Tool (BLAST®) (Altschul et al. 1990 J. Mol. Biol. 215: 403-10), which is available from several sources, including the National Center for Biotechnology Information (NCBI, Bethesda, Md.) and on the Internet, for use in connection with the sequence analysis programs blastp, blastn, blastx, tblastn and tblastx. It can be accessed on the internet via the National Library of Medicine (NLM)'s world-wide-web URL. A description of how to determine sequence identity using this program is available at the NLM's website on BLAST tutorial. Another example of a mathematical algorithm utilized for the global comparison of sequences is the Clustal W and Clustal X (Larkin et al. 2007 Bioinformatics, 23, 2947-294, Clustal W and Clustal X version 2.0) as well as Clustal Omega. Unless otherwise stated, references to sequence identity used herein refer to BLAST®.

“Short day” is a 24 hour period (a day) with less than 12 hours of light.

“Strawberries” are plants whose fruits are juicy, edible, low growing, and of genus Fragaria. According to this present disclosure, a strawberry is the desired product to be harvested from a plug plant grown at low elevation.

“Tissue culture” refers to a composition comprising isolated cells of the same or a different type or a collection of such cells organized into parts of a plant. Exemplary types of tissue cultures are protoplasts, calli, plant clumps, and plant cells that can generate tissue culture that are intact in plants or parts of plants, such as embryos, pollen, flowers, seeds, leaves, stems, roots, root tips, anthers, pistils, meristematic cells, axillary buds, ovaries, seed coat, endosperm, hypocotyls, cotyledons and the like. Means for preparing and maintaining plant tissue culture are well known in the art. By way of example, a tissue culture comprising organs has been used to produce regenerated plants. U.S. Pat. Nos. 5,750,870, 5,959,185, 5,973,234, and 5,977,445 describe certain techniques, the disclosures of which are incorporated herein by reference.

“Vernalization” is the process of promoting flowering by exposing plants to prolonged chilling and/or controlled photoperiods. The process of vernalization may also be referred to as ‘conditioning’.

“Yield” refers to the weight of fruit harvested. Yield can be measured by the number of fruit, weight of fruit, or the number and weight of fruit harvested per plant, or per acre of plants, within a given period of time, such as a season or a year.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); Cell and Tissue Culture: Laboratory Procedures in Biotechnology (Doyle & Griffiths, John Wiley & Sons 1998); and Current Protocols in Molecular Biology (Ausubel et al. eds., John Wiley & Sons 2003), including supplements 1-117, the disclosures of which are incorporated herein by reference.

### Overview

The present disclosure relates to a cultivated Rosaceae plant, such as strawberries, blackberries, and raspberries, having an early flowering trait that can lead to increased yields resulting from genetically engineered Terminal Flowering alleles (TFL1) or homologs thereof, wherein one or more TFL1 alleles has one or more edits that reduce or knockout protein function. The disclosure further teaches methods of producing the same.

### The Terminal Flower Gene

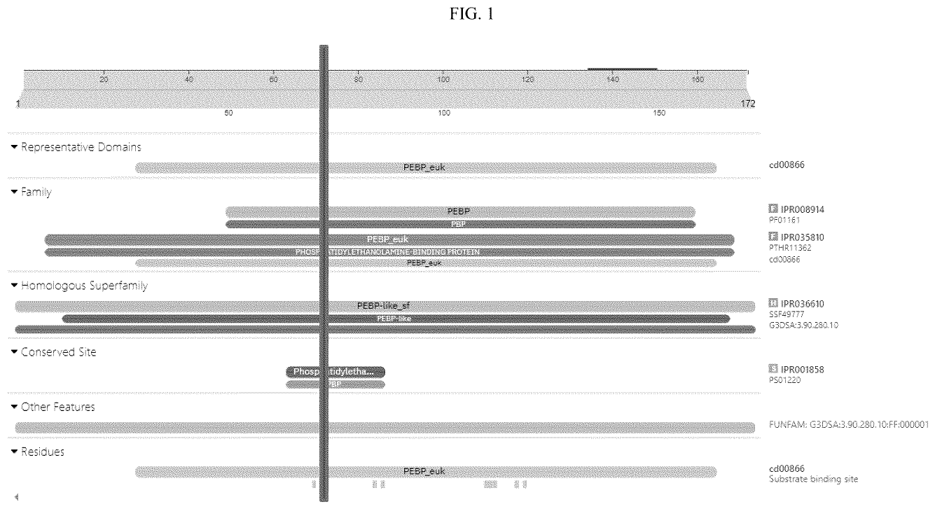

The Terminal Flower gene, “TFL” is part of the PEBP Superfamily (FIG. 1). Members of the PEBP family bind different substrates including phospholipids, opioids, and hydrophobic odorant molecules as well as having different oligomerization states (monomer/dimer/tetramer) (J. Khosa, et al., PHOSPHATIDYLETHANOLAMINE-BINDING PROTEINS: the conductors of dual reproduction in plants with vegetative storage organs, Journal of Experimental Botany, Volume 72, Issue 8, 2 Apr. 2021, Pages 2845-2856).

Unlike the Flowering Locus T gene, “FT”, which encodes a protein that functions as an activator of flowering (J Plant Physiol. 2015 Apr. 1; 177:60-6), TFL1 has been shown to function as a suppressor of flowering. In Rosaceae plants like strawberry, expression of endogenous TFL1 is regulated by temperature and photoperiod. For example, in short-day varieties, conditioning the strawberry plants at low temperature and with a short-day photoperiod reduces expression of TFL1.

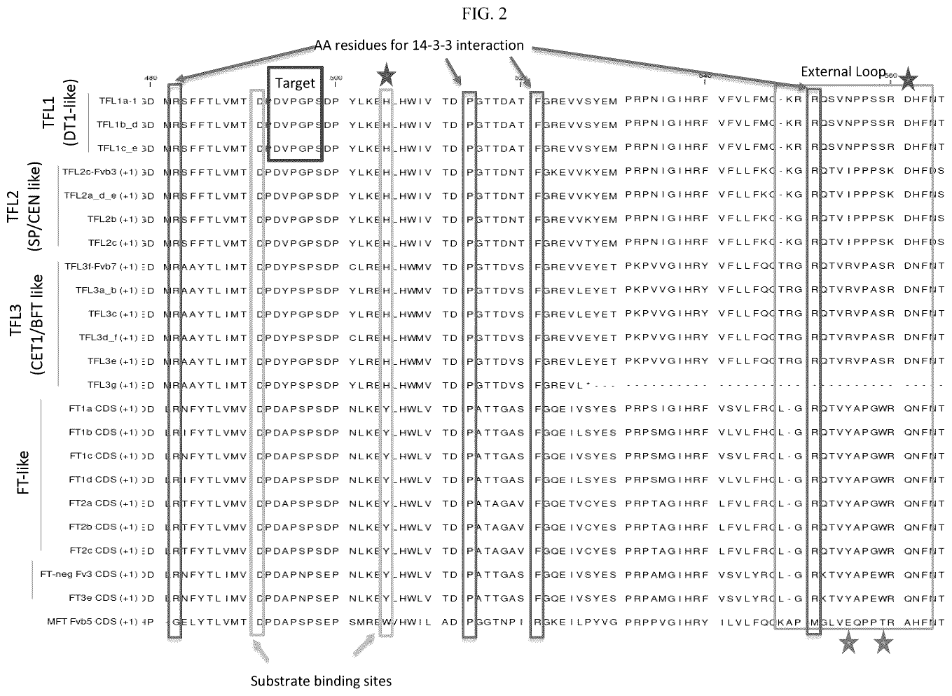

To inhibit flowering, TFL1 forms a floral repressive complex with 14-3-3 proteins and FD proteins, which brings TFL into proximity with DNA. Taoka, Ki., et al. 2011 found four amino acids, R64, P96, F103 and R132 in FT that interact with the 14-3-3 proteins in rice (Taoka, Ki., Ohki, I., Tsuji, H. et al. 14-3-3 proteins act as intracellular receptors for rice Hd3a florigen. Nature 476, 332-335 (2011). These four amino acids are conserved in TFL (FIG. 2).

For an additional summary of the flowering time signaling pathway, see Bradley et al., (1997) Science 275:80-83; Ruiz-Garcia et al. (1997) Plant Cell 9:1921-1934; Corbesier and Coupland, (2005) Plant Cell and Environment 28:54-66; Mandel and Yanofsky, (1995) Nature 377:522-524; Weigel and Nilsson, (1995) Nature 377:495-500; Kardailsky et al., (1999) Science 286:1962-1965; Iwata et al., (2012) The Plant J. 69:116-125; Koskela et al., (2012) Plant Physiol. 159:1043-1054; and Nakano et al., (2015) J. Plant Physiol. 177:60-66.

Fragaria, known as strawberry is a genus of flowering plants in the rose family, Rosaceae. As used herein, the term strawberry encompasses plant species in the Fragaria genus. The genetics of strawberry plants are uniquely diverse in terms of ploidy. Strawberry plant species can be diploid, tetraploid, pentaploid, hexaploid, heptaploid, octoploid, or decaploid (which have 2, 4, 5, 6, 7, 8, or 10 sets of chromosomes, respectively). Some species of Fragaria have uncategorized ploidy. Table 1 below shows TFL1 allele copy number in different types of strawberry.

TABLE 1
TFL1 copy number in different strawberry varieties

| Photoperiod | Variety | TFL1a | TFL1b | TFL1d | TFL1c | TFL1e | Total |
|---|---|---|---|---|---|---|---|
| Short day | S1 | 1 | 3 | 2 | 0 | 2 | 8 |
| Short day | S2 | 1 | 2 | 2 | 1 | 1 | 7 |
| Short day | S3 | 2 | 2 | 2 | 0 | 2 | 8 |
| Short day | S4 | 1 | 2 | 2 | 1 | 1 | 7 |
| Short day | S5 | 0 | 2 | 2 | 1 | 1 | 6 |
| Short day | CM | 1 | 2 | 2 | 0 | 2 | 7 |
| Short day | LF9 | 2 | 2 | 2 | 0 | 2 | 8 |
| CF: Day Neutral | N1* | 2 | 3 | 2* | 0 | 2 | 9 |
| CF: Everbearer | E1 | 2 | 2 | 2 | 2 | 0 | 8 |
| CF | H4 | 0 | 0 | 0 | 0 | 2 | 2 |

*most similar to TFL1d allele
CF=Continual Flowering

As shown above, all varieties except S5 have at least one copy of TFL1a. All varieties except H4 have at least two copies of both TFL1b and TFL1d. Lastly, all varieties have two copies of TFLc or TFLe, or, one copy each of TFLc and TFLe.

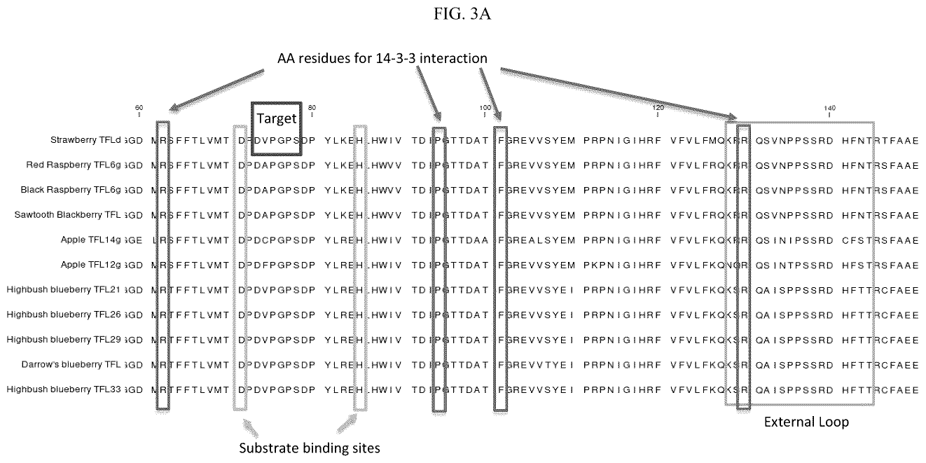

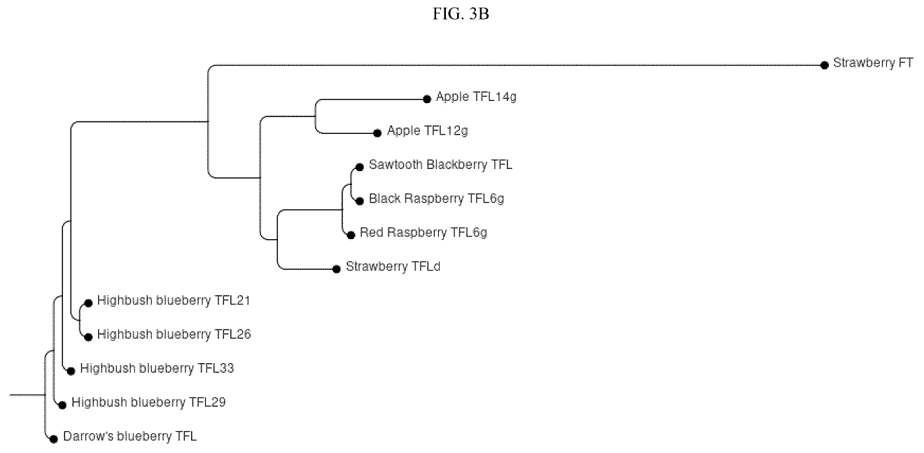

FIG. 3A shows a partial protein alignment of TFL1 amino acid sequences across different genera and species within Rosaceae compared to strawberry TFL1d; phylogenetic tree shown in FIG. 3B. Sequence ID numbers for each and the % identity shared with strawberry TFL1d are shown below in Table 2.

TABLE 2
Percent Identity of TFL1d amino acid sequences in different berry crops

| SEQ | | | |
|---|---|---|---|
| ID | | | |
| NO: | % Identity AA sequence | Strawberry_TFL1d | Strawberry_FT |
| 117 | Strawberry_FT | 60% | 100% |
| 112 | Highbush_Blueberry_TFL21 | 84% | 61% |
| 113 | Highbush_Blueberry_TFL26 | 84% | 61% |
| 115 | Darrow's_Blueberry_TFL | 83% | 61% |
| 114 | Highbush_Blueberry_TFL29 | 84% | 61% |
| 116 | Highbush_Blueberry_TFL33 | 71% | 52% |
| 106 | Strawberry_TFL1d | 100% | 60% |
| 107 | Red Raspberry_TFL6g | 92% | 62% |
| 108 | Black Raspberry_TFL6g | 92% | 62% |
| 109 | Sawtooth_Blackberry_TFL | 92% | 62% |
| 110 | Apple_TFL14g | 87% | 59% |
| 111 | Apple_TFL12g | 90% | 58% |

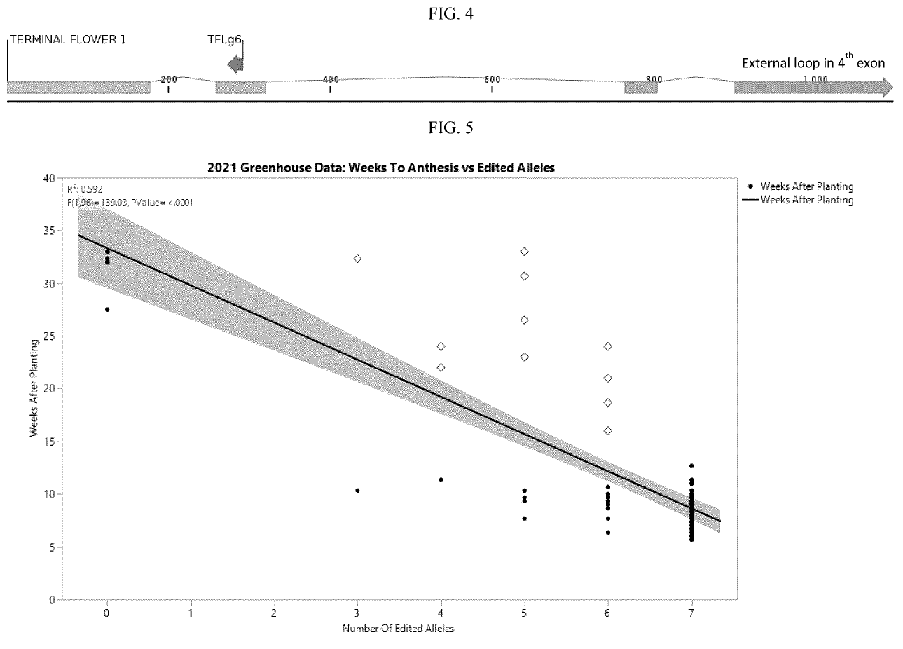

TFL1 has 4 exons (FIG. 4). The arrow of FIG. 4 labeled “TFLg6” was the region targeted herein (see also the boxes labeled “target” in FIGS. 2 and 3A) for genomic editing, and lies between two substrate binding sites.

In some embodiments, the disclosure relates to a cultivated Rosaceae plant, wherein an unedited TFL1d wild-type allele encodes a protein that shares at least 70%, at least 75%, at least 80%, at least 85%, at least 90%, at least about 91%, at least about 92%, at least about 93%, at least about 94%, at least about 95%, at least about 96%, at least about 97%, at least about 98%, or at least 98.5%, or at least about 99%, or at least 99.5%, or at least 99.8%, or at least 99.9% sequence identity with SEQ ID NO: 62.

In some embodiments, the disclosure relates to a cultivated Rosaceae plant, wherein an unedited TFL1d wild-type allele encodes a protein that shares between 70% and 100% sequence identity, between 75% and 100% sequence identity, between 80% and 100% sequence identity, between 85% and 100% sequence identity, between 86% and 100% sequence identity, between 87% and 100% sequence identity, between 88% and 100% sequence identity, between 89% and 100% sequence identity, between 90% and 100% sequence identity, between 91% and 100% sequence identity, between 92% and 100% sequence identity, between 93% and 100% sequence identity, between 94% and 100% sequence identity, between 95% and 100% sequence identity, between 96% and 100% sequence identity, between 97% and 100% sequence identity, between 98% and 100% sequence identity, or between 99% and 100% sequence identity with SEQ ID NO: 62.

In some embodiments, the unedited TFL1d wild-type allele encodes a protein that shares between 99.1% and 100% sequence identity, between 99.2% and 100% sequence identity, between 99.3% and 100% sequence identity, between 99.4% and 100% sequence identity, between 99.5% and 100% sequence identity, between 99.6% and 100% sequence identity, between 99.7% and 100% sequence identity, between 99.8% and 100% sequence identity, or between 99.9% and 100% sequence identity with SEQ ID NO: 62.

In some embodiments, the disclosure relates to a cultivated Rosaceae plant, wherein an unedited TFL1d wild-type allele shares at least 70%, at least 75%, at least 80%, at least 85%, at least 90%, at least about 91%, at least about 92%, at least about 93%, at least about 94%, at least about 95%, at least about 96%, at least about 97%, at least about 98%, or at least 98.5%, or at least about 99%, or at least 99.5%, or at least 99.8%, or at least 99.9% sequence identity with SEQ ID NO: 21.

In some embodiments, the disclosure relates to a cultivated Rosaceae plant, wherein an unedited TFL1d wild-type allele shares between 70% and 100% sequence identity, between 75% and 100% sequence identity, between 80% and 100% sequence identity, between 85% and 100% sequence identity, between 86% and 100% sequence identity, between 87% and 100% sequence identity, between 88% and 100% sequence identity, between 89% and 100% sequence identity, between 90% and 100% sequence identity, between 91% and 100% sequence identity, between 92% and 100% sequence identity, between 93% and 100% sequence identity, between 94% and 100% sequence identity, between 95% and 100% sequence identity, between 96% and 100% sequence identity, between 97% and 100% sequence identity, between 98% and 100% sequence identity, or between 99% and 100% sequence identity with SEQ ID NO: 21.

In some embodiments, the unedited TFL1d wild-type allele shares between 99.1% and 100% sequence identity, between 99.2% and 100% sequence identity, between 99.3% and 100% sequence identity, between 99.4% and 100% sequence identity, between 99.5% and 100% sequence identity, between 99.6% and 100% sequence identity, between 99.7% and 100% sequence identity, between 99.8% and 100% sequence identity, or between 99.9% and 100% sequence identity with SEQ ID NO: 21.

### Targeted Genome Editing to Reduce TFL1d Function

Emerging genome editing technology provides the opportunity to establish non-transgenic traits, which substantially reduces costs and development times.

The disclosure provides novel, engineered TFL1 proteins. As used herein, the term “engineered” refers to a non-natural DNA, protein, cell, or organism that would not normally be found in nature and was created by human intervention. An “engineered protein” refers to a protein whose amino acid sequence was conceived of and created in the laboratory using one or more of the techniques of biotechnology, protein design, or protein engineering, such as molecular biology, protein biochemistry, bacterial transformation, plant transformation, site-directed mutagenesis, directed evolution using random mutagenesis, genome editing, gene editing, gene cloning, DNA ligation, DNA synthesis, protein synthesis, and DNA shuffling. For example, an engineered protein may have one or more deletions, insertions, or substitutions relative to the coding sequence of the wild-type protein and each deletion, insertion, or substitution takes place on one or more amino acids. In some embodiments, the engineered proteins are genetically engineered with a targeted genome or gene editing system such as CRISPR-Cas system described below.

In some embodiments, provided are novel, engineered TFL1 proteins that decrease the time to flower (or, increase flower earliness) and increase yield. In some aspects, the novel, engineered TFL1 proteins are TFL1d proteins. In some aspects, the early flowering trait is independent of vernalization. Further provided are methods of making plants comprising engineered TFL1 alleles and plants produced therefrom.

In some embodiments, genetically modified cultivated Rosaceae plants of the present disclosure can be grown directly in a production field without a prior growth season of vernalization. By this method, the time necessary for plant production is reduced. In some embodiments, the time necessary for plant production is reduced by at least 0.5 month, 1 month, 2 months, 3 months, 4 months, 5 months, 5.5 months, 6 months, 6.5 months, 7 months, 8 months, or more.

Because of the degeneracy of the genetic code, a variety of different DNA sequences can encode the altered or engineered proteins disclosed herein. DNA sequences encoding TFL1d with the amino acid substitutions, deletions, and insertions described herein can be produced by introducing mutations into the DNA sequence encoding a wild-type TFL1d allele using methods known in the art. It is well within the capability of one of skill in the art to create alternative DNA sequences encoding the same, or essentially the same, altered or engineered proteins as described herein. These variant or alternative DNA sequences are within the scope of the embodiments described herein. As used herein, references to “essentially the same” sequence refers to sequences which encode amino acid substitutions, deletions, additions, or insertions that do not materially alter the functional activity of the protein encoded by the DNA molecule of the embodiments described herein. Allelic variants of the nucleotide sequences encoding a wild-type or engineered protein are also encompassed within the scope of the embodiments described herein.

The above referenced genomic alterations may be achieved by any number of means well known in art, for example by genome modification using site-specific integration or genome editing. Targeted modification of plant genomes through the use of genome editing methods can be used to create improved plant lines through modification of plant genomic DNA. As used herein “site-directed integration” or “site-specific integration” refers to genome editing methods the enable targeted insertion of one or more nucleic acids of interest into a plant genome. Suitable methods for altering a wild-type DNA sequence or a preexisting transgenic sequence or for inserting DNA into a plant genome at a pre-determined chromosomal site include any method known in the art. Exemplary methods include the use of sequence specific nucleases, such as zinc-finger nucleases, engineered or native meganucleases, TALE-endonucleases, or an RNA-guided endonucleases (for example, a Clustered Regularly Interspersed Short Palindromic Repeat (CRISPR)/Cas9 system, a CRISPR/Cpf1 system, a CRISPR/CasX system, a CRISPR/CasY system, a CRISPR/Cascade system). Several embodiments relate to methods of genome editing by using single-stranded oligonucleotides to introduce precise base pair modifications in a plant genome, as described by Sauer et al., Plant Physiology 170(4): 1917-1928 (2016). Methods of genome editing to modify, delete, or insert nucleic acid sequences into genomic DNA are known in the art.

In some embodiments, the disclosure relates to plants and plant parts of a cultivated Rosaceae plant having genetically engineered TFL1d1 and TFL1d2 alleles, or homologs thereof, wherein each TFL1d allele has one or more edits that reduce protein function. In some embodiments, the Rosaceae plant or plant part is a species of Fragaria.

In some embodiments, the disclosure relates to a cultivated Fragaria plant having an early flowering trait, wherein said early flowering trait is caused by genetically engineered TFL1d1 and TFL1d2 alleles, and wherein each TFL1d allele has one or more edits that reduce protein function.

In some embodiments, the disclosure relates to a cultivated Fragaria plant having an early flowering trait, wherein said early flowering trait is caused by genetically engineered TFL1d1 and TFL1d2 alleles, and wherein each TFL1d allele has one or more edits in exon 2.

In some embodiments, the disclosure relates to a cultivated Fragaria plant having an early flowering trait, wherein said early flowering trait is caused by genetically engineered TFL1d1 and TFL1d2 alleles, and wherein each TFL1d allele has one or more edits that disrupt TFL protein interaction with a 14-3-3 protein.

In some embodiments, the disclosure relates to a cultivated Fragaria plant having an early flowering trait, wherein said early flowering trait is caused by genetically engineered TFL1d1 and TFL1d2 alleles, and wherein each TFL1d allele has one or more edits that disrupt the TFL proteins substrate binding.

In some aspects, the targeted edits of TFL1d1 and TFL1d2 are combined with one or more edits in another TFL1 allele.

TABLE 3
Combinations of TFL1 allele edits with
TFL1d1 and TFL1d2 in Fragaria sp.

| | | | | | | |
|---|---|---|---|---|---|---|
| TFL1d1 | TFL1d2 | | | | | |
| TFL1d1 | TFL1d2 | TFL1a | | | | |
| TFL1d1 | TFL1d2 | TFL1b1 | | | | |
| TFL1d1 | TFL1d2 | TFL1b2 | | | | |
| TFL1d1 | TFL1d2 | TFL1c | | | | |
| TFL1d1 | TFL1d2 | TFL1e | | | | |
| TFL1d1 | TFL1d2 | TFL1a | TFL1b1 | | | |
| TFL1d1 | TFL1d2 | TFL1a | TFL1b2 | | | |
| TFL1d1 | TFL1d2 | TFL1a | TFL1c | | | |
| TFL1d1 | TFL1d2 | TFL1a | TFL1e | | | |
| TFL1d1 | TFL1d2 | TFL1b1 | TFL1b2 | | | |
| TFL1d1 | TFL1d2 | TFL1b1 | TFL1c | | | |
| TFL1d1 | TFL1d2 | TFL1b1 | TFL1e | | | |
| TFL1d1 | TFL1d2 | TFL1b2 | TFL1c | | | |
| TFL1d1 | TFL1d2 | TFL1b2 | TFL1e | | | |
| TFL1d1 | TFL1d2 | TFL1c | TFL1e | | | |
| TFL1d1 | TFL1d2 | TFL1a | TFL1b1 | TFL1b2 | | |
| TFL1d1 | TFL1d2 | TFL1a | TFL1b1 | TFL1c | | |
| TFL1d1 | TFL1d2 | TFL1a | TFL1b1 | TFL1e | | |
| TFL1d1 | TFL1d2 | TFL1a | TFL1b2 | TFL1c | | |
| TFL1d1 | TFL1d2 | TFL1a | TFL1b2 | TFL1e | | |
| TFL1d1 | TFL1d2 | TFL1a | TFL1c | TFL1e | | |
| TFL1d1 | TFL1d2 | TFL1b1 | TFL1b2 | TFL1c | | |
| TFL1d1 | TFL1d2 | TFL1b1 | TFL1b2 | TFL1e | | |
| TFL1d1 | TFL1d2 | TFL1b1 | TFL1c | TFL1e | | |
| TFL1d1 | TFL1d2 | TFL1b2 | TFL1c | TFL1e | | |
| TFL1d1 | TFL1d2 | TFL1a | TFL1b1 | TFL1b2 | TFL1c | |
| TFL1d1 | TFL1d2 | TFL1a | TFL1b1 | TFL1b2 | TFL1e | |
| TFL1d1 | TFL1d2 | TFL1a | TFL1b1 | TFL1c | TFL1e | |
| TFL1d1 | TFL1d2 | TFL1a | TFL1b2 | TFL1c | TFL1e | |
| TFL1d1 | TFL1d2 | TFL1b1 | TFL1b2 | TFL1c | TFL1e | |
| TFL1d1 | TFL1d2 | TFL1a | TFL1b1 | TFL1b2 | TFL1c | TFL1e |

In some aspects, the techniques described herein relate to a cultivated Fragaria sp. plant having an early flowering trait, wherein said early flowering trait is caused by genetically engineered TFL1d1, TFL1d2, and TFL1a alleles.

In some aspects, the techniques described herein relate to a cultivated Fragaria sp. plant having an early flowering trait, wherein said early flowering trait is caused by genetically engineered TFL1d1, TFL1d2, and TFL1b1 alleles.

In some aspects, the techniques described herein relate to a cultivated Fragaria sp. plant having an early flowering trait, wherein said early flowering trait is caused by genetically engineered TFL1d1, TFL1d2, and TFL1b2 alleles.

In some aspects, the techniques described herein relate to a cultivated Fragaria sp. plant having an early flowering trait, wherein said early flowering trait is caused by genetically engineered TFL1d1, TFL1d2, and TFL1c alleles.

In some aspects, the techniques described herein relate to a cultivated Fragaria sp. plant having an early flowering trait, wherein said early flowering trait is caused by genetically engineered TFL1d1, TFL1d2, and TFL1e alleles.

In some aspects, the techniques described herein relate to a cultivated Fragaria sp. plant having an early flowering trait, wherein said early flowering trait is caused by genetically engineered TFL1d1, TFL1d2, TFL1a, and TFL1b1 alleles.

In some aspects, the techniques described herein relate to a cultivated Fragaria sp. plant having an early flowering trait, wherein said early flowering trait is caused by genetically engineered TFL1d1, TFL1d2, TFL1a, and TFL1b2 alleles.

In some aspects, the techniques described herein relate to a cultivated Fragaria sp. plant having an early flowering trait, wherein said early flowering trait is caused by genetically engineered TFL1d1, TFL1d2, TFL1a, and TFL1c alleles.

In some aspects, the techniques described herein relate to a cultivated Fragaria sp. plant having an early flowering trait, wherein said early flowering trait is caused by genetically engineered TFL1d1, TFL1d2, TFL1a, and TFL1e alleles.

In some aspects, the techniques described herein relate to a cultivated Fragaria sp. plant having an early flowering trait, wherein said early flowering trait is caused by genetically engineered TFL1d1, TFL1d2, TFL1b1, and TFL1b2 alleles.

In some aspects, the techniques described herein relate to a cultivated Fragaria sp. plant having an early flowering trait, wherein said early flowering trait is caused by genetically engineered TFL1d1, TFL1d2, TFL1b1, and TFL1c alleles.

In some aspects, the techniques described herein relate to a cultivated Fragaria sp. plant having an early flowering trait, wherein said early flowering trait is caused by genetically engineered TFL1d1, TFL1d2, TFL1b1, and TFL1e alleles.

In some aspects, the techniques described herein relate to a cultivated Fragaria sp. plant having an early flowering trait, wherein said early flowering trait is caused by genetically engineered TFL1d1, TFL1d2, TFL1b2, and TFL1c alleles.

In some aspects, the techniques described herein relate to a cultivated Fragaria sp. plant having an early flowering trait, wherein said early flowering trait is caused by genetically engineered TFL1d1, TFL1d2, TFL1b2, and TFL1e alleles.

In some aspects, the techniques described herein relate to a cultivated Fragaria sp. plant having an early flowering trait, wherein said early flowering trait is caused by genetically engineered TFL1d1, TFL1d2, TFL1c, and TFL1e alleles.

In some aspects, the techniques described herein relate to a cultivated Fragaria sp. plant having an early flowering trait, wherein said early flowering trait is caused by genetically engineered TFL1d1, TFL1d2, TFL1a, TFL1b1, and TFL1b2 alleles.

In some aspects, the techniques described herein relate to a cultivated Fragaria sp. plant having an early flowering trait, wherein said early flowering trait is caused by genetically engineered TFL1d1, TFL1d2, TFL1a, TFL1b1, and TFL1c alleles.

In some aspects, the techniques described herein relate to a cultivated Fragaria sp. plant having an early flowering trait, wherein said early flowering trait is caused by genetically engineered TFL1d1, TFL1d2, TFL1a, TFL1b1, and TFL1e alleles.

In some aspects, the techniques described herein relate to a cultivated Fragaria sp. plant having an early flowering trait, wherein said early flowering trait is caused by genetically engineered TFL1d1, TFL1d2, TFL1a, TFL1b2, and TFL1c alleles.

In some aspects, the techniques described herein relate to a cultivated Fragaria sp. plant having an early flowering trait, wherein said early flowering trait is caused by genetically engineered TFL1d1, TFL1d2, TFL1a, TFL1b2, and TFL1e alleles.

In some aspects, the techniques described herein relate to a cultivated Fragaria sp. plant having an early flowering trait, wherein said early flowering trait is caused by genetically engineered TFL1d1, TFL1d2, TFL1a, TFL1c, and TFL1e alleles.

In some aspects, the techniques described herein relate to a cultivated Fragaria sp. plant having an early flowering trait, wherein said early flowering trait is caused by genetically engineered TFL1d1, TFL1d2, TFL1b1, TFL1b2, and TFL1c alleles.

In some aspects, the techniques described herein relate to a cultivated Fragaria sp. plant having an early flowering trait, wherein said early flowering trait is caused by genetically engineered TFL1d1, TFL1d2, TFL1b1, TFL1b2, and TFL1e alleles.

In some aspects, the techniques described herein relate to a cultivated Fragaria sp. plant having an early flowering trait, wherein said early flowering trait is caused by genetically engineered TFL1d1, TFL1d2, TFL1b1, TFL1c, and TFL1e alleles.

In some aspects, the techniques described herein relate to a cultivated Fragaria sp. plant having an early flowering trait, wherein said early flowering trait is caused by genetically engineered TFL1d1, TFL1d2, TFL1b2, TFL1c, and TFL1e alleles.

In some aspects, the techniques described herein relate to a cultivated Fragaria sp. plant having an early flowering trait, wherein said early flowering trait is caused by genetically engineered TFL1d1, TFL1d2, TFL1a, TFL1b1, TFL1b2, and TFL1c alleles.

In some aspects, the techniques described herein relate to a cultivated Fragaria sp. plant having an early flowering trait, wherein said early flowering trait is caused by genetically engineered TFL1d1, TFL1d2, TFL1a, TFL1b1, TFL1b2, and TFL1e alleles.

In some aspects, the techniques described herein relate to a cultivated Fragaria sp. plant having an early flowering trait, wherein said early flowering trait is caused by genetically engineered TFL1d1, TFL1d2, TFL1a, TFL1b1, TFL1c, and TFL1e alleles.

In some aspects, the techniques described herein relate to a cultivated Fragaria sp. plant having an early flowering trait, wherein said early flowering trait is caused by genetically engineered TFL1d1, TFL1d2, TFL1a, TFL1b2, TFL1c, and TFL1e alleles.

In some aspects, the techniques described herein relate to a cultivated Fragaria sp. plant having an early flowering trait, wherein said early flowering trait is caused by genetically engineered TFL1d1, TFL1d2, TFL1b1, TFL1b2, TFL1c, and TFL1e alleles.

In some aspects, the techniques described herein relate to a cultivated Fragaria sp. plant having an early flowering trait, wherein said early flowering trait is caused by genetically engineered TFL1d1, TFL1d2, TFL1a, TFL1b1, TFL1b2, TFL1c, and TFL1e alleles.

In some aspects, the techniques described herein relate to a cultivated Fragaria sp. plant having an early flowering trait, wherein said early flowering trait is caused by at least one genetically engineered TFL1d allele, wherein the engineered TFL1d allele includes any one of SEQ ID NOs: 1-20, SEQ ID NOs: 22-41, or sequences at least 75% identical thereto. In some aspects, the techniques described herein relate to a cultivated Fragaria sp. plant having an early flowering trait, wherein said early flowering trait is caused by at least one genetically engineered TFL1d allele, wherein the engineered TFL1d allele includes any one of SEQ ID NOs: 1-20, SEQ ID NOs: 22-41, or sequences at least 80% identical thereto. In some aspects, the techniques described herein relate to a cultivated Fragaria sp. plant having an early flowering trait, wherein said early flowering trait is caused by at least one genetically engineered TFL1d allele, wherein the engineered TFL1d allele includes any one of SEQ ID NOs: 1-20, SEQ ID NOs: 22-41, or sequences at least 85% identical thereto. In some aspects, the techniques described herein relate to a cultivated Fragaria sp. plant having an early flowering trait, wherein said early flowering trait is caused by at least one genetically engineered TFL1d allele, wherein the engineered TFL1d allele includes any one of SEQ ID NOs: 1-20, SEQ ID NOs: 22-41, or sequences at least 90% identical thereto. In some aspects, the techniques described herein relate to a cultivated Fragaria sp. plant having an early flowering trait, wherein said early flowering trait is caused by at least one genetically engineered TFL1d allele, wherein the engineered TFL1d allele includes any one of SEQ ID NOs: 1-20, SEQ ID NOs: 22-41, or sequences at least 95% identical thereto. In some aspects, the techniques described herein relate to a cultivated Fragaria sp. plant having an early flowering trait, wherein said early flowering trait is caused by at least one genetically engineered TFL1d allele, wherein the engineered TFL1d allele includes any one of SEQ ID NOs: 1-20, SEQ ID NOs: 22-41, or sequences sharing between 90% and 99.9% identity thereto.

In some aspects, the techniques described herein relate to a cultivated Fragaria sp. plant having an early flowering trait, wherein said early flowering trait is caused by at least one genetically engineered TFL1d allele, wherein the at least one engineered TFL1d allele is selected from SEQ ID NOs: 1-2, and sequences at least 75% identical thereto.

In some aspects, the techniques described herein relate to a cultivated Fragaria sp. plant having an early flowering trait, wherein said early flowering trait is caused by at least one genetically engineered TFL1d allele, wherein the at least one engineered TFL1d allele is selected from SEQ ID NOs: 3-4, and sequences at least 75% identical thereto.

In some aspects, the techniques described herein relate to a cultivated Fragaria sp. plant having an early flowering trait, wherein said early flowering trait is caused by at least one genetically engineered TFL1d allele, wherein the at least one engineered TFL1d allele is selected from SEQ ID NOs: 5-6, and sequences at least 75% identical thereto.

In some aspects, the techniques described herein relate to a cultivated Fragaria sp. plant having an early flowering trait, wherein said early flowering trait is caused by at least one genetically engineered TFL1d allele, wherein the at least one engineered TFL1d allele is selected from SEQ ID NOs: 7-8, and sequences at least 75% identical thereto.

In some aspects, the techniques described herein relate to a cultivated Fragaria sp. plant having an early flowering trait, wherein said early flowering trait is caused by at least one genetically engineered TFL1d allele, wherein the at least one engineered TFL1d allele is selected from SEQ ID NOs: 9-10, and sequences at least 75% identical thereto.

In some aspects, the techniques described herein relate to a cultivated Fragaria sp. plant having an early flowering trait, wherein said early flowering trait is caused by at least one genetically engineered TFL1d allele, wherein the at least one engineered TFL1d allele is selected from SEQ ID NOs: 11-12, and sequences at least 75% identical thereto.

In some aspects, the techniques described herein relate to a cultivated Fragaria sp. plant having an early flowering trait, wherein said early flowering trait is caused by at least one genetically engineered TFL1d allele, wherein the at least one engineered TFL1d allele is selected from SEQ ID NOs: 13-14, and sequences at least 75% identical thereto.

In some aspects, the techniques described herein relate to a cultivated Fragaria sp. plant having an early flowering trait, wherein said early flowering trait is caused by at least one genetically engineered TFL1d allele, wherein the at least one engineered TFL1d allele is selected from SEQ ID NOs: 15-16, and sequences at least 75% identical thereto.

In some aspects, the techniques described herein relate to a cultivated Fragaria sp. plant having an early flowering trait, wherein said early flowering trait is caused by at least one genetically engineered TFL1d allele, wherein the at least one engineered TFL1d allele is selected from SEQ ID NOs: 17-18, and sequences at least 75% identical thereto.

In some aspects, the techniques described herein relate to a cultivated Fragaria sp. plant having an early flowering trait, wherein said early flowering trait is caused by at least one genetically engineered TFL1d allele, wherein the at least one engineered TFL1d allele is selected from SEQ ID NOs: 19-20, and sequences at least 75% identical thereto.

In some aspects, the techniques described herein relate to a cultivated Fragaria sp. plant having an early flowering trait, wherein said early flowering trait is caused by at least one genetically engineered TFL1d allele, wherein the at least one engineered TFL1d allele is selected from SEQ ID NOs: 22-23, and sequences at least 75% identical thereto.

In some aspects, the techniques described herein relate to a cultivated Fragaria sp. plant having an early flowering trait, wherein said early flowering trait is caused by at least one genetically engineered TFL1d allele, wherein the at least one engineered TFL1d allele is selected from SEQ ID NOs: 24-25, and sequences at least 75% identical thereto.

In some aspects, the techniques described herein relate to a cultivated Fragaria sp. plant having an early flowering trait, wherein said early flowering trait is caused by at least one genetically engineered TFL1d allele, wherein the at least one engineered TFL1d allele is selected from SEQ ID NOs: 26-27, and sequences at least 75% identical thereto.

In some aspects, the techniques described herein relate to a cultivated Fragaria sp. plant having an early flowering trait, wherein said early flowering trait is caused by at least one genetically engineered TFL1d allele, wherein the at least one engineered TFL1d allele is selected from SEQ ID NOs: 28-29, and sequences at least 75% identical thereto.

In some aspects, the techniques described herein relate to a cultivated Fragaria sp. plant having an early flowering trait, wherein said early flowering trait is caused by at least one genetically engineered TFL1d allele, wherein the at least one engineered TFL1d allele is selected from SEQ ID NOs: 30-31, and sequences at least 75% identical thereto.

In some aspects, the techniques described herein relate to a cultivated Fragaria sp. plant having an early flowering trait, wherein said early flowering trait is caused by at least one genetically engineered TFL1d allele, wherein the at least one engineered TFL1d allele is selected from SEQ ID NOs: 32-33, and sequences at least 75% identical thereto.

In some aspects, the techniques described herein relate to a cultivated Fragaria sp. plant having an early flowering trait, wherein said early flowering trait is caused by at least one genetically engineered TFL1d allele, wherein the at least one engineered TFL1d allele is selected from SEQ ID NOs: 34-35, and sequences at least 75% identical thereto.

In some aspects, the techniques described herein relate to a cultivated Fragaria sp. plant having an early flowering trait, wherein said early flowering trait is caused by at least one genetically engineered TFL1d allele, wherein the at least one engineered TFL1d allele is selected from SEQ ID NOs: 36-37, and sequences at least 75% identical thereto.

In some aspects, the techniques described herein relate to a cultivated Fragaria sp. plant having an early flowering trait, wherein said early flowering trait is caused by at least one genetically engineered TFL1d allele, wherein the at least one engineered TFL1d allele is selected from SEQ ID NOs: 38-39, and sequences at least 75% identical thereto.

In some aspects, the techniques described herein relate to a cultivated Fragaria sp. plant having an early flowering trait, wherein said early flowering trait is caused by at least one genetically engineered TFL1d allele, wherein the at least one engineered TFL1d allele is selected from SEQ ID NOs: 40-41, and sequences at least 75% identical thereto.

In some aspects, the techniques described herein relate to a cultivated Fragaria sp. plant having an early flowering trait, wherein said early flowering trait is caused by at least one genetically engineered TFL1d allele, wherein the at least one engineered TFL1d allele encodes any one of SEQ ID NOs: 42-61, SEQ ID NOs: 63-82, or sequences at least 75% identical thereto. In some aspects, the techniques described herein relate to a cultivated Fragaria sp. plant having an early flowering trait, wherein said early flowering trait is caused by at least one genetically engineered TFL1d allele, wherein the at least one engineered TFL1d allele encodes any one of SEQ ID NOs: 42-61, SEQ ID NOs: 63-82, or sequences at least 80% identical thereto. In some aspects, the techniques described herein relate to a cultivated Fragaria sp. plant having an early flowering trait, wherein said early flowering trait is caused by at least one genetically engineered TFL1d allele, wherein the at least one engineered TFL1d allele encodes any one of SEQ ID NOs: 42-61, SEQ ID NOs: 63-82, or sequences at least 85% identical thereto. In some aspects, the techniques described herein relate to a cultivated Fragaria sp. plant having an early flowering trait, wherein said early flowering trait is caused by at least one genetically engineered TFL1d allele, wherein the at least one engineered TFL1d allele encodes any one of SEQ ID NOs: 42-61, SEQ ID NOs: 63-82, or sequences at least 90% identical thereto. In some aspects, the techniques described herein relate to a cultivated Fragaria sp. plant having an early flowering trait, wherein said early flowering trait is caused by at least one genetically engineered TFL1d allele, wherein the at least one engineered TFL1d allele encodes any one of SEQ ID NOs: 42-61, SEQ ID NOs: 63-82, or sequences at least 95% identical thereto. In some aspects, the techniques described herein relate to a cultivated Fragaria sp. plant having an early flowering trait, wherein said early flowering trait is caused by at least one genetically engineered TFL1d allele, wherein the at least one engineered TFL1d allele encodes any one of SEQ ID NOs: 42-61, SEQ ID NOs: 63-82, and sequences sharing between 90% and 99.9% identity thereto.

In some aspects, the techniques described herein relate to a cultivated Fragaria sp. plant having an early flowering trait, wherein said early flowering trait is caused by at least one genetically engineered TFL1d allele, wherein the at least one engineered TFL1d allele encodes a sequence selected from SEQ ID NOs: 42-43, and sequences at least 75% identical thereto.

In some aspects, the techniques described herein relate to a cultivated Fragaria sp. plant having an early flowering trait, wherein said early flowering trait is caused by at least one genetically engineered TFL1d allele, wherein the at least one engineered TFL1d allele encodes a sequence selected from SEQ ID NOs: 44-45, and sequences at least 75% identical thereto.

In some aspects, the techniques described herein relate to a cultivated Fragaria sp. plant having an early flowering trait, wherein said early flowering trait is caused by at least one genetically engineered TFL1d allele, wherein the at least one engineered TFL1d allele encodes a sequence selected from SEQ ID NOs: 46-47, and sequences at least 75% identical thereto.

In some aspects, the techniques described herein relate to a cultivated Fragaria sp. plant having an early flowering trait, wherein said early flowering trait is caused by at least one genetically engineered TFL1d allele, wherein the at least one engineered TFL1d allele encodes a sequence selected from SEQ ID NOs: 48-49, and sequences at least 75% identical thereto.

In some aspects, the techniques described herein relate to a cultivated Fragaria sp. plant having an early flowering trait, wherein said early flowering trait is caused by at least one genetically engineered TFL1d allele, wherein the at least one engineered TFL1d allele encodes a sequence selected from SEQ ID NOs: 50-51, and sequences at least 75% identical thereto.

In some aspects, the techniques described herein relate to a cultivated Fragaria sp. plant having an early flowering trait, wherein said early flowering trait is caused by at least one genetically engineered TFL1d allele, wherein the at least one engineered TFL1d allele encodes a sequence selected from SEQ ID NOs: 52-53, and sequences at least 75% identical thereto.

In some aspects, the techniques described herein relate to a cultivated Fragaria sp. plant having an early flowering trait, wherein said early flowering trait is caused by at least one genetically engineered TFL1d allele, wherein the at least one engineered TFL1d allele encodes a sequence selected from SEQ ID NOs: 54-55, and sequences at least 75% identical thereto.

In some aspects, the techniques described herein relate to a cultivated Fragaria sp. plant having an early flowering trait, wherein said early flowering trait is caused by at least one genetically engineered TFL1d allele, wherein the at least one engineered TFL1d allele encodes a sequence selected from SEQ ID NOs: 56-57, and sequences at least 75% identical thereto.

In some aspects, the techniques described herein relate to a cultivated Fragaria sp. plant having an early flowering trait, wherein said early flowering trait is caused by at least one genetically engineered TFL1d allele, wherein the at least one engineered TFL1d allele encodes a sequence selected from SEQ ID NOs: 58-59, and sequences at least 75% identical thereto.

In some aspects, the techniques described herein relate to a cultivated Fragaria sp. plant having an early flowering trait, wherein said early flowering trait is caused by at least one genetically engineered TFL1d allele, wherein the at least one engineered TFL1d allele encodes a sequence selected from SEQ ID NOs: 60-61, and sequences at least 75% identical thereto.

In some aspects, the techniques described herein relate to a cultivated Fragaria sp. plant having an early flowering trait, wherein said early flowering trait is caused by at least one genetically engineered TFL1d allele, wherein the at least one engineered TFL1d allele encodes a sequence selected from SEQ ID NOs: 63-64, and sequences at least 75% identical thereto.

In some aspects, the techniques described herein relate to a cultivated Fragaria sp. plant having an early flowering trait, wherein said early flowering trait is caused by at least one genetically engineered TFL1d allele, wherein the at least one engineered TFL1d allele encodes a sequence selected from SEQ ID NOs: 65-66, and sequences at least 75% identical thereto.

In some aspects, the techniques described herein relate to a cultivated Fragaria sp. plant having an early flowering trait, wherein said early flowering trait is caused by at least one genetically engineered TFL1d allele, wherein the at least one engineered TFL1d allele encodes a sequence selected from SEQ ID NOs: 67-68, and sequences at least 75% identical thereto.

In some aspects, the techniques described herein relate to a cultivated Fragaria sp. plant having an early flowering trait, wherein said early flowering trait is caused by at least one genetically engineered TFL1d allele, wherein the at least one engineered TFL1d allele encodes a sequence selected from SEQ ID NOs: 69-70, and sequences at least 75% identical thereto.

In some aspects, the techniques described herein relate to a cultivated Fragaria sp. plant having an early flowering trait, wherein said early flowering trait is caused by at least one genetically engineered TFL1d allele, wherein the at least one engineered TFL1d allele encodes a sequence selected from SEQ ID NOs: 71-72, and sequences at least 75% identical thereto.

In some aspects, the techniques described herein relate to a cultivated Fragaria sp. plant having an early flowering trait, wherein said early flowering trait is caused by at least one genetically engineered TFL1d allele, wherein the at least one engineered TFL1d allele encodes a sequence selected from SEQ ID NOs: 73-74, and sequences at least 75% identical thereto.

In some aspects, the techniques described herein relate to a cultivated Fragaria sp. plant having an early flowering trait, wherein said early flowering trait is caused by at least one genetically engineered TFL1d allele, wherein the at least one engineered TFL1d allele encodes a sequence selected from SEQ ID NOs: 75-76, and sequences at least 75% identical thereto.

In some aspects, the techniques described herein relate to a cultivated Fragaria sp. plant having an early flowering trait, wherein said early flowering trait is caused by at least one genetically engineered TFL1d allele, wherein the at least one engineered TFL1d allele encodes a sequence selected from SEQ ID NOs: 77-78, and sequences at least 75% identical thereto.

In some aspects, the techniques described herein relate to a cultivated Fragaria sp. plant having an early flowering trait, wherein said early flowering trait is caused by at least one genetically engineered TFL1d allele, wherein the at least one engineered TFL1d allele encodes a sequence selected from SEQ ID NOs: 79-80, and sequences at least 75% identical thereto.

In some aspects, the techniques described herein relate to a cultivated Fragaria sp. plant having an early flowering trait, wherein said early flowering trait is caused by at least one genetically engineered TFL1d allele, wherein the at least one engineered TFL1d allele encodes a sequence selected from SEQ ID NOs: 81-82, and sequences at least 75% identical thereto.

In some aspects, the techniques described herein relate to a cultivated Fragaria sp. plant, wherein the one or more edits include an insertion in exon 2 of TFL1d. In some aspects, the techniques described herein relate to a cultivated Fragaria sp. plant, wherein the one or more edits include a deletion in exon 2 of TFL1d. In some aspects, the techniques described herein relate to a cultivated Fragaria sp. plant, wherein the one or more edits to TFL1d result in a frameshift mutation. In some aspects, the techniques described herein relate to a cultivated Fragaria sp. plant, wherein the one or more edits to TFL1d result in an early stop codon. In some aspects, the techniques described herein relate to a cultivated Fragaria sp. plant, wherein the one or more edits to TFL1d include a complete deletion of exon 2. In some aspects, the techniques described herein relate to a cultivated Fragaria sp. plant, wherein the one or more edits to each TFL1d allele include a combination of insertions and deletions in exon 2.

In some aspects, the cultivated Fragaria sp. plants described herein having targeted non-functional edits in TFL1d alleles flower at least one week earlier than a cultivated Fragaria sp. plant of the same variety having wildtype TFL1d1 and TFL1d2 alleles when grown under the same environmental conditions. In some aspects, the plant flowers at least two weeks earlier, at least three weeks earlier, at least four weeks earlier, at least five weeks earlier, at least six weeks earlier, at least seven weeks earlier, at least eight weeks earlier, at least nine weeks earlier, at least 10 weeks earlier, at least 11 weeks earlier, at least 12 weeks earlier, at least 13 weeks earlier, at least 14 weeks earlier, at least 15 weeks earlier, at least 16 weeks earlier, at least 17 weeks earlier, at least 18 weeks earlier, at least 19 weeks earlier, or at least 20 weeks earlier than a cultivated Fragaria sp. plant of the same variety having wildtype TFL1d1 and TFL1d2 alleles when grown under the same environmental conditions.

In some aspects, the cultivated Fragaria sp. plants described herein having targeted non-functional edits in TFL1d alleles flower between six and 12 weeks earlier, between 12 and 20 weeks, or between 15 and 19 weeks earlier than a cultivated Fragaria sp. plant of the same variety having wildtype TFL1d1 and TFL1d2 alleles when grown under the same environmental conditions.

In some aspects, the cultivated Fragaria sp. plants described herein having targeted non-functional edits in TFL1d alleles produce between 1% and 10% increase in yield, between 10% and 25% increase in yield, between 25% and 50% increase in yield, between 50% and 100% increase in yield, or between 100% and 500% increase in yield compared to a cultivated Fragaria sp. plant of the same variety having wildtype TFL1d1 and TFL1d2 alleles when grown under the same environmental conditions.

In some embodiments, the methods disclosed herein increase fruit production of the plants. In some embodiments, the yield is measured by fruit weight produced per plant, or fruit number per plant, or per acre, within a given period of time, such as per season or per year. In some embodiments, the yield is increased by at least about 5%, about 10%, about 15%, about 20%, about 25%, about 30%, about 40%, about 50%, about 60%, about 70%, about 80%, about 90%, about one time, about two times, about three times, or more.

### Methods of Generating Plants Described Herein

Methods for modify gene activity that can be utilized in the present disclosure include, but are not limited to, mutagenesis (for example, chemical mutagenesis, radiation mutagenesis, transposon mutagenesis, insertional mutagenesis, signature tagged mutagenesis, site-directed mutagenesis, and natural mutagenesis), knock-outs/knock-ins, antisense, RNA interference, and any other suitable methods known to a skilled artisan, such as Zinc finger nuclease (ZFN) technology (ZFN-1, ZFN-2 and ZFN-3, see U.S. Pat. No. 9,145,565, incorporated by reference in its entirety), Oligonucleotide directed mutagenesis (ODM), Cisgenesis and intragenesis, RNA-dependent DNA methylation (RdDM), Grafting (on GM rootstock), Reverse breeding, Agro-infiltration (agro-infiltration “sensu stricto”, agro-inoculation, floral dip), Transcription Activator-Like Effector Nucleases (TALENs, see U.S. Pat. Nos. 8,586,363 and 9,181,535, incorporated by reference in their entireties), the CRISPR/Cas system (see U.S. Pat. Nos. 8,697,359; 8,771,945; 8,795,965; 8,865,406; 8,871,445; 8,889,356; 8,895,308; 8,906,616; 8,932,814; 8,945,839; 8,993,233; and 8,999,641, which are all hereby incorporated by reference), engineered meganuclease re-engineered homing endonucleases, DNA guided genome editing (Gao et al., Nature Biotechnology (2016), doi: 10.1038/nbt.3547, incorporated by reference in its entirety), and Synthetic genomics. For more information of gene modification in plants, such as agents, protocols, see Acquaah et al. (Principles of plant genetics and breeding, Wiley-Blackwell, 2007, ISBN 1405136464, 9781405136464, which is herein incorporated by reference in its entity).

In some embodiments, the gene activity is modified by introducing a mutation into the plants. Methods of introducing a mutation into an endogenous gene or replacing an endogenous gene or a portion thereof with a mutant gene are well known in the art. In certain embodiments, a variety of DNA nucleases may be utilized to introduce mutations into an endogenous gene. In certain embodiments, the DNA nuclease is deficient in its nuclease activity. In certain embodiments, the enzyme is a Zinc-finger nuclease. In further embodiments, the Zinc-finger nuclease is ZF-FokI or ZF-Tn3. In certain embodiments, the enzyme is a transcription activator-like effector nuclease (TALEN). In further embodiments, the TALEN is TAL-FokI. In certain embodiments, the enzyme is a homing endonuclease. In further embodiments, the homing endonuclease is LAGLIDADG, GIY-YIG, His-Cys, H-N-H, PD-(D/E)xK, or Vsr-like. In certain embodiments, the enzyme is an RNA-guided nuclease such as a Clustered Regularly Interspaced Short Palindromic Repeats (CRISPR) enzyme. In further embodiments, the CRISPR enzyme is a type II CRISPR enzyme. In further embodiments, the type II CRISPR enzyme is Cas9. In certain embodiments, the CRISPR enzyme is deficient in its nuclease activity. In certain embodiments, various DNA integrases may be utilized to introduce mutations into an endogenous gene or replace an endogenous gene with a mutant gene. In certain embodiments, the DNA integrase is λ-int or ϕC31. In certain embodiments, a DNA recombinase may be utilized to introduce mutations into an endogenous gene or replace an endogenous gene with a mutant gene. In certain embodiments, the DNA recombinase is Cre, Flp, or RMCE. In other embodiments, the Cas9 peptide can include one or more of the mutations described in the literature, including but not limited to the functional mutations described in: Fonfara et al. (2014) Nucleic Acids Res. 42(4):2577-90; Nishimasu H. et al. (2014) Cell. 156(5):935-49; Jinek M. et al. (2012) Science 337:816-21; and Jinek M. et al. (2014) Science 343(6176); see also U.S. patent application Ser. No. 13/842,859, filed Mar. 15, 2013, which is hereby incorporated by reference; further, see U.S. Pat. Nos. 8,697,359; 8,771,945; 8,795,965; 8,865,406; 8,871,445; 8,889,356; 8,895,308; 8,906,616; 8,932,814; 8,945,839; 8,993,233; and 8,999,641, which are all hereby incorporated by reference.

In some embodiments, the activity of one or more TFL1 alleles is disrupted by using an inhibitory nucleotide sequence, such as nucleotide sequences for RNA interference, antisense oligonucleotides, microRNA, and/or steric-blocking oligonucleotides (See Kole et al., (2012) Drug Discovery 11:125-140; Ossowski et al. (2008) The Plant Journal, 53(4):674-690; Wang et al. (2002) Current Opinion in Plant Biology, 5(2):146-150; Vaucheret et al. (2001) Journal of Cell Science 114:3083-3091; Stam et al. (1997) Annals of Botany 79(1):3-12; Schwab et al. (2006) The Plant Cell 18(5):1121-1133; C. David Allis et al., Epigenetics, CSHL Press (2007) ISBN 10: 0879697245, ISBN 13: 978087969724; Sohail et al., Gene silencing by RNA interference: technology and application, CRC Press (2005) ISBN 0849321417, 9780849321412; Engelke et al., RAN Interference, Academic Press (2005) ISBN 0121827976, 9780121827977; and Doran et al., RNA Interference: Methods for Plants and Animals, CABI (2009) ISBN 1845934105, 9781845934101, each of which is incorporated herein by reference in its entirety for all purposes). In some embodiments one or more TFL1 alleles are disrupted by RNA interference (RNAi). RNAi is the process of sequence-specific, post-transcriptional gene silencing or transcriptional gene silencing in animals and plants, initiated by double-stranded RNA (dsRNA) that is homologous in sequence to the silenced gene. The preferred RNA effector molecules useful in this disclosure must be sufficiently distinct in sequence from any host polynucleotide sequences for which function is intended to be undisturbed after any of the methods of this disclosure are performed. Computer algorithms may be used to define the essential lack of homology between the RNA molecule polynucleotide sequence and host, essential, normal sequences.

In some embodiments, one or more TFL1 alleles are disrupted by double-strand RNA. The term “dsRNA” or “dsRNA molecule” or “double-strand RNA effector molecule” refers to an at least partially double-strand ribonucleic acid molecule containing a region of at least about 19 or more nucleotides that are in a double-strand conformation. The double-stranded RNA effector molecule may be a duplex double-stranded RNA formed from two separate RNA strands, or it may be a single RNA strand with regions of self-complementarity capable of assuming an at least partially double-stranded hairpin conformation (for example, a hairpin dsRNA or stem-loop dsRNA). In various embodiments, the dsRNA consists entirely of ribonucleotides or consists of a mixture of ribonucleotides and deoxynucleotides, such as RNA/DNA hybrids. The dsRNA may be a single molecule with regions of self-complementarity such that nucleotides in one segment of the molecule base pair with nucleotides in another segment of the molecule. In one aspect, the regions of self-complementarity are linked by a region of at least about 3-4 nucleotides, or about 5, 6, 7, 9 to 15 nucleotides or more, which lacks complementarity to another part of the molecule and thus remains single-stranded (for example, the “loop region”). Such a molecule will assume a partially double-stranded stem-loop structure, optionally, with short single stranded 5′ and/or 3′ ends. In one aspect the regions of self-complementarity of the hairpin dsRNA or the double-stranded region of a duplex dsRNA will comprise an Effector Sequence and an Effector Complement (for example, linked by a single-stranded loop region in a hairpin dsRNA). The Effector Sequence or Effector Strand is that strand of the double-stranded region or duplex which is incorporated in or associates with RISC. In one aspect the double-stranded RNA effector molecule will comprise an at least 19 contiguous nucleotide effector sequence, preferably 19 to 29, 19 to 27, or 19 to 21 or more nucleotides, which is a reverse complement to the RNA of the target gene, or an opposite strand replication intermediate. In some embodiments, the dsRNA effector molecule of the disclosure is a “hairpin dsRNA”, a “dsRNA hairpin”, “short-hairpin RNA” or “shRNA”, for example, an RNA molecule of less than approximately 400 to 500 nucleotides (nt), or less than 100 to 200 nt, in which at least one stretch of at least 15 to 100 nucleotides (for example, 17 to 50 nt, 19 to 29 nt) is based paired with a complementary sequence located on the same RNA molecule (single RNA strand), and where said sequence and complementary sequence are separated by an unpaired region of at least about 4 to 7 nucleotides (or about 9 to about 15 nt, about 15 to about 100 nt, about 100 to about 1000 nt) which forms a single-stranded loop above the stem structure created by the two regions of base complementarity. The shRNA molecules comprise at least one stem-loop structure comprising a double-stranded stem region of about 17 to about 500 bp; about 17 to about 50 bp; about 40 to about 100 bp; about 18 to about 40 bp; or from about 19 to about 29 bp; homologous and complementary to a target sequence to be inhibited; and an unpaired loop region of at least about 4 to 7 nucleotides, or about 9 to about 15 nucleotides, about 15 to about 100 nt, about 250-500 bp, about 100 to about 1000 nt, which forms a single-stranded loop above the stem structure created by the two regions of base complementarity. It will be recognized, however, that it is not strictly necessary to include a “loop region” or “loop sequence” because an RNA molecule comprising a sequence followed immediately by its reverse complement will tend to assume a stem-loop conformation even when not separated by an irrelevant “stuffer” sequence. In yet another embodiment, the RNA interference is through use of an RNA “trigger”, such as described in U.S. Patent Application Publications US20140215656; US20130067618A1; US20130288895A1; US20130254940A1; US20130097726; US20130326731A1, all of which are incorporated herein in their entirety.

In some embodiments, the RNAi construct may comprise one or more inverted repeats. The inverted repeats can be transcribed into interference RNA molecules in the plants. In some embodiments, the transcribed interference RNA molecules can target the promoter region, the coding region, the intron, the 5′ UTR region, and/or the 3′ UTR region of one or more TFL1 alleles.

In some embodiments, the inverted repeats comprise a sense strand and an anti-sense strand. In some embodiments, the sense stand and the anti-sense stand are perfectly complementary to each other. In some embodiments, the sense stand and the anti-sense stand are not perfectly complementary to each other for the full length, but are at least complementary partially. In some embodiments, the sense stand shares about 70%, about 80%, about 90%, about 95%, about 99% or more homology to the targeted gene.

In some aspects, the techniques described herein relate to a method for increasing yield in a cultivated Rosaceae plant, the method including: targeting one or more TFL1 alleles to reduce or knockout TFL1 function, wherein at least one of the targeted TFL1 alleles shares 80% or more sequence identity with SEQ ID NO: 62. In some aspects, the targeted TFL1 allele shares 85%, 90%, 95%, 96%, 97%, 98%, 99% or more sequence identity with SEQ ID NO: 62. In some aspects, the targeted TFL1 allele shares between 80% and 85%, between 85% and 90%, between 90 and 95%, or between 95% and 100% sequence identity with SEQ ID NO: 62.

In some aspects, the techniques described herein relate to a method for inducing an early flowering trait in a cultivated Rosaceae plant, the method including: targeting one or more TFL1 alleles to reduce or knockout TFL1 function, wherein at least one of the targeted TFL1 alleles shares 80% or more sequence identity with SEQ ID NO: 62. In some aspects, the targeted TFL1 allele shares 85%, 90%, 95%, 96%, 97%, 98%, 99% or more sequence identity with SEQ ID NO: 62. In some aspects, the targeted TFL1 allele shares between 80% and 85%, between 85% and 90%, between 90 and 95%, or between 95% and 100% sequence identity with SEQ ID NO: 62.

In some aspects, the techniques described herein relate to a method, wherein the targeting one or more TFL1 alleles results in: reduced gene expression level, reduced gene copy number, reduced gene amplification, reduced RNA activity level, reduced mRNA abundance, reduced mRNA synthesis rate, reduced mRNA stability, reduced protein activity level, reduced protein synthesis, reduced protein abundance, reduced protein stability, reduced substrate binding, reduced interaction with a 14-3-3 protein, or a combination thereof.

In some aspects, the techniques described herein relate to a method, wherein the targeting is RNA interference (RNAi), genome editing, or mutation of the endogenous TFL1 gene.

In some aspects, the techniques described herein relate to a method, wherein the RNA interference is induced by expression in a cell of the cultivated Rosaceae plant an RNAi cassette targeting the endogenous TFL1 gene, or by topical application of RNAi triggers targeting the endogenous TFL1 gene.

In some aspects, the techniques described herein relate to a method, wherein the genome editing is by expression in a cell of the cultivated Rosaceae plant of a zinc-finger nuclease, a TALE-mediated nuclease, or an RNA-guided nuclease.

In some aspects, the techniques described herein relate to a method, wherein mutation of the endogenous TFL1 gene is by chemical mutagenesis, radiation mutagenesis, transposon mutagenesis, insertional mutagenesis, signature tagged mutagenesis, site-directed mutagenesis, and/or natural mutagenesis.

In some aspects, the techniques described herein relate to a method, wherein the targeting includes editing a region corresponding to between M60 and E150 of SEQ ID NO: 62.

In some aspects, the techniques described herein relate to a method, wherein the targeting includes editing a region in exon 2 corresponding to between V67 and W87 of SEQ ID NO: 62.

In some aspects, the techniques described herein relate to a method, wherein all copies of a TFL1d1 allele or TFL1d1 homolog are targeted.

### Genome Editing by CRISPR

Conventional approaches to engineer new traits rely either on mutation breeding or introduction of novel genes into the genomes of crop species by transformation. Conventional plant transformation methods deliver exogenous DNA that integrates into the genome at random locations. Thus, to identify and isolate transgenic lines with desirable traits, it is necessary to generate and screen thousands of random-integration events. Using genome editing, DNA can be modified in a targeted way providing new alternatives to develop novel traits in plants.

Genome editing by CRISPR, which stands for Clustered Regularly Interspaced Short Palindromic Repeats, is based on a natural immune process used by bacteria to defend themselves against invading viruses. Indeed, in bacteria the invading viral DNA will be cut through use of a guide RNA (gRNA), or piece of RNA, and a CRISPR-associated protein (Cas). The last step of the bacterial immune process, when the gRNA is combined with Cas and cleaves the target DNA, has been adopted for genome editing in laboratories.

There are at least three main CRISPR system types (Type I, II, and III) and at least 10 distinct subtypes (Makarova, K. S., et.al., Nat Rev Microbiol. 2011 May 9; 9(6):467-477). Type I and III systems use Cas protein complexes and short guide polynucleotide sequences to target selected DNA regions. Type II systems rely on a single protein (e.g. Cas9) and the targeting guide polynucleotide, where a portion of the 5′ end of a guide sequence is complementary to a target nucleic acid. For more information on the CRISPR gene editing compositions and methods of the present disclosure, see U.S. Pat. Nos. 8,697,359; 8,889,418; 8,771,945; and 8,871,445, each of which is hereby incorporated in its entirety for all purposes.

CRISPR genome editing requires two components, a gRNA and a nuclease, such as the Cas enzyme or a Cas-like enzyme. These components associate to form a ribonucleoprotein (RNP) complex, where after the gRNA can base pair with a complementary protospacer sequence (i.e. the target genomic sequence of about 20 bases in length) under the condition that a particular adjacent sequence, called a protospacer-adjacent motif (PAM), is present in the genome. The PAM is only a few bases long, and its sequence depends on the type of nuclease used. Once the gRNA binds to the target DNA (protospacer), the nuclease (e.g. Cas enzyme) recognizes this complex and makes a precise cut at the target site.

Either Cas9 or Cas12a (also called Cpf1) or other Cas-like enzymes can be used to cleave target DNA, resulting in a Double Strand Break (DSB). Each Cas enzyme is directed by the gRNA to a user-specified cut site in the genome. Like Cas9 nucleases, Cas12a1 family members contain a RuvC-like endonuclease domain, but lack the second HNH endonuclease domain of Cas9. Cas12a cleaves DNA in a staggered pattern in contrast to Cas9 which produces a blunt-end. Moreover, for cleavage Cas12a requires only one RNA rather than the two tracrRNA and crRNA needed by Cas9. For Cas9 as well as Cas12a, the target sequence of the gRNAs must be next to a PAM sequence. In the case of Cas9, the PAM sequence corresponds to NGG, where N is any base. The gRNA will recognize and bind to 20 nucleotides on the DNA strand opposite from the NGG PAM site. For Cas12a, the PAM sequence is TTTV, where V can represent A, C, or G. Using Alt-R Cas12a Ultra from Integrated DNA Technologies, a TTTT PAM sequence may also work. The “V” of the TTTV is immediately adjacent to the base at the 5′ end of the non-targeted strand side of the protospacer element. The guide RNA for Cas12a is relatively short and is approximately 40 to 44 bases long.

The damage caused by the double strand break (DSB) will be repaired in eukaryotic cells, primarily by two pathways: Non-Homologous End-Joining (NHEJ) and Homology Directed Repair (HDR). The HDR mechanism requires the presence of a donor DNA template containing regions of homology to both sites of the DNA break. This donor DNA can carry specific mutations and is delivered simultaneously with a preassembled Cas RNP complex composed of Cas9 or Cas12a and synthetically produced gRNAs.

Altogether, targeted cleavage events induced by nucleases can be used to introduce targeted mutations (deletions, substitutions and insertions) in genomic DNA sequences and as such, can be used as an efficient tool for genome editing in plants.

Examples of endonucleases include, but are not limited to, meganuclease, a zinc-finger nuclease (ZFN), a transcription activator-like effector nucleases (TALEN), an Argonaute (non-limiting examples of Argonaute proteins include Thermus thermophilus Argonaute (TtAgo), Pyrococcus furiosus Argonaute (PfAgo), Natronobacterium gregoryi Argonaute (NgAgo), an RNA-guided nuclease, such as a CRISPR associated nuclease (non-limiting examples of CRISPR associated nucleases include Cas1, Cas1B, Cas2, Cas3, Cas4, Cas5, Cas6, Cas7, Cas8, Cas9 (also known as Csn1 and Csx12), Cas12a (also known as Cpf1), Mad7 (also known as ErCas12a), Cas1O, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, CasX, CasY, homologs thereof, or modified versions thereof.

In some embodiments, the disclosure relates to a method of producing a plant, plant part, or plant cell having increased yield, the method comprising: transforming a plant, plant part, or plant cell with the recombinant, engineered polynucleotide taught herein. In some embodiments, the method comprises transforming the plant with one or more RNPs comprising a guide RNA and nuclease described herein. In some embodiments, the method comprises transforming a plant with a DNA construct comprising a guide RNA and nuclease described herein. In some aspects, the guide RNA and nuclease are provided in a ribonucleoprotein (RNP) complex. In some aspects, the method is plasmid-free.

In some embodiments, the disclosure relates to a method of producing a plant, plant part, or plant cell having an early flowering trait, the method comprising: transforming a plant, plant part, or plant cell with the recombinant, engineered polynucleotide taught herein. In some embodiments, the method comprises transforming the plant with one or more RNPs comprising a guide RNA and nuclease described herein. In some embodiments, the method comprises transforming a plant with a DNA construct comprising a guide RNA and nuclease described herein. In some aspects, the guide RNA and nuclease are provided in a ribonucleoprotein (RNP) complex. In some aspects, the method is plasmid-free.

In some aspects, plants comprising one or more of the genetic alterations described herein may be selfed or crossed to produce lines that are homozygous for one or more of the genetic alterations described herein. In some aspects, the genetic alterations described herein may be transferred or introgressed to other varieties through conventional breeding schemes.

In some embodiments, the plant breeding techniques are selected from the group consisting of recurrent selection, mass selection, hybridization, open-pollination, backcrossing, pedigree breeding, mutation breeding, haploid/double haploid production, and marker enhanced selection. In some embodiments, the plant breeding technique is mutation breeding and the mutation selected is spontaneous or artificially induced.

Rosaceae Plants for Use with the Disclosed Methods

In some aspects, the methods and plants described herein relate to a cultivated Rosaceae plant.

In some embodiments, the cultivated Rosaceae is a species of Fragaria. Fragaria species include but are not limited to, 1) diploid: F. bucharica, F. chinensis, F. daltoniana, F. gracilis, F. hayatai, F. iinumae, F. nilgerrensis, F. nipponica, F. nubicola, F. pentaphylla, F. rubicola, F. vesca, F. viridis, F. vezoensis, and F. x bifera; 2) tetraploid: F. corymbosa, F. moupinensis, F. orientalis, and F. tibetica; 3) pentaploid: F. x bringhurstii; 4) hexaploid: F. moschata; 5) hexaploid: F. moschata; 6) heptaploid: F. x comarum; 7) octaploid: F. x ananassa, F. chiloensis, F. chiloensis subsp. chiloensis forma chiloensis, F. chiloensis subsp. chiloensis forma patagonica, F. chiloensis subsp. Lucida, F. chiloensis subsp. Pacifica, Fragaria chiloensis subsp. Sandwicensis, F. iturupensis, F. ovalis, and F. virginiana; 8) decaploid: C. frutescens, C. chinense, and C. pendulum. More Fragaria species are described in Liston et al. (Fragaria: A genus with deep historical roots and ripe for evolutionary and ecological insights, American Journal of Botany (2014) 101:1686-1699) and Kole (Wild Crop relatives: Genomic and Breeding Resources: Temperate Fruits, Chapter 2 Fragaria, 2011).

In some the cultivated Fragaria sp. plant is a June-bearing variety, an early season June-bearing variety, an early midseason June-bearing variety, a midseason June-bearing variety, a late midseason June-bearing variety, a late season June-bearing variety, a short-day variety, a seasonal flowering variety, a long-day variety, a day-neutral variety, a perpetual flowering variety, a recurrent variety, a remontant variety, a long-day variety, or an everbearing variety.

In some aspects, the plant is selected from F. iinumae, F. nipponica, F. pentaphylla, F. vesca, F. viridis, F. moupinensis, F. orientalis, F. moschata, F. chiloensis, F. iturupensis, F. virginiana, F. cascadensis, F. x ananassa, and hybrids thereof. In some aspects, the plant is selected from F. x ananassa, F. x bringhurstii, and F. x vescana.

In some embodiments, the cultivated Rosaceae plant is a species of Rubus. In some aspects, the plant is selected from R. idaeus, R. allegheniensis, R. occidentalis, R. argutus, R. ursinus, R. laciniatus, R. ulmifolius, R. leucodermis, R. strigosus, R. ellipticus, R. subsp. rubus, and hybrids thereof.

In some embodiments, the cultivated Rosaceae plant is a species of Vaccinium. In some aspects, the plant is selected from V. corymbosum, V. darrowii, V. angustifolium, V. ashei, and hybrids thereof.

In some embodiments, the cultivated Rosaceae plant is a species of Malus. In some aspects, the techniques described herein relate to a method, wherein the plant is selected from M. domestica, and hybrids thereof.

In some embodiments, plants in which one or more TFL1 alleles are modified have one or more agriculturally important traits. As used herein, “agronomically important traits” include any phenotype in a plant or plant part that is useful or advantageous for human use. Examples of agronomically important traits include but are not limited to those that result in increased biomass production, increased food production, improved food quality, increased fruit production. Additional examples of agronomically important traits includes pest resistance, vigor, development time (time to harvest), enhanced nutrient content, novel growth patterns, flavors or colors, salt, heat, drought and cold tolerance, disease resistance, fruit size, fruit weight, fruit color, fruit nutrients, fruit taste, and the like. Non-limited examples of disease resistance include, resistant to Raspberry ringspot virus (RpRSV), Strawberry crinkle virus (SCV), Strawberry feather leaf virus, Strawberry latent “C” virus (SLCV), Strawberry latent ringspot virus (SLRSV), Strawberry leaf roll virus, Strawberry mild yellow edge virus (SMYEV), Strawberry mottle virus (SMV), Strawberry pallidosis virus, Strawberry vein banding virus (SVBV), Tobacco necrosis virus (TNV), Tobacco ringspot virus (TRSV), Tobacco streak virus (TSV), Strawberry necrotic shock virus (SNSV), Tomato black ring virus (TBRV), Tomato bushy stunt virus (TBSV), Tomato ringspot virus (ToRSV), and Xanthamonas fragariae (angular leafspot). Additional preferred traits are described in Yue et al. (An Evaluation of U.S. Strawberry Producers Trait Prioritization: Evidence from Audience Surveys, HortScience 49(2) 188-193 (2014)).

The present disclosure also provides methods for breeding strawberry plants which have engineered TFL1d alleles. In some embodiments, the methods comprise (i) crossing any one of the plants of the present disclosure comprising a modified gene as a donor to a recipient plant to create a F1 population; (ii) evaluating the phenotypes in the offspring derived from said F1 population; and (iii) selecting offspring that have prolonged flowering time. In some embodiments, the recipient plant is an elite line having one or more certain agronomically important traits.

The most common method for the introduction of new genetic material into a plant genome involves the use of living cells of the bacterial pathogen Agrobacterium tumefaciens to literally inject a piece of DNA, called transfer or T-DNA, into individual plant cells (usually following wounding of the tissue) where it is targeted to the plant nucleus for chromosomal integration. There are numerous patents governing Agrobacterium mediated transformation and particular DNA delivery plasmids designed specifically for use with Agrobacterium, for example, U.S. Pat. No. 4,536,475, EP0265556, EP0270822, WO8504899, WO8603516, U.S. Pat. No. 5,591,616, EP0604662, EP0672752, WO8603776, WO9209696, WO9419930, WO9967357, U.S. Pat. No. 4,399,216, WO8303259, U.S. Pat. No. 5,731,179, EP068730, WO9516031, U.S. Pat. Nos. 5,693,512, 6,051,757 and EP904362A1. Agrobacterium-mediated plant transformation involves as a first step the placement of DNA fragments cloned on plasmids into living Agrobacterium cells, which are then subsequently used for transformation into individual plant cells. Agrobacterium-mediated plant transformation is thus an indirect plant transformation method. Methods of Agrobacterium-mediated plant transformation that involve using vectors with plant derived border sequences are also well known to those skilled in the art and can have applicability in the present disclosure. See, for example, U.S. Pat. No. 7,250,554, which is incorporated herein by reference in its entirety.

Nehra et al. (1990) Plant Cell Rep. 9:293-298) and James et al. (1990) Acta Horticulturae 280:495-502) describe methods for Agrobacterium-mediated transformation of strawberry either via callus or leaf disk regeneration system. Since then, further research on regeneration and transformation via Agrobacterium tumefaciens have been performed in different combinations of growth regulators and culture conditions using various strawberry cultivars since the success of transformation was cultivar-dependent. U.S. Pat. No. 6,274,791 describes methods for Agrobacterium-mediated transformation and regeneration of strawberry plants. See, for example, U.S. Pat. No. 6,274,791, which is incorporated herein by reference in its entirety.

Traditional methods for breeding strawberry plants can be utilized to create additional strawberry plants based on the present disclosure, such as those described in Strawberry; History, Breeding and Physiology by Darrow GM (1966), and U.S. Pat. No. 6,598,339 which is hereby incorporated by reference in its entirety. The cultivated strawberry (F. x ananassa) is an interspecific hybrid between the wild octaploid species F. chiloensis L. and F. virginiana Duch., which was first introduced in the 1750s (Darrow, 1966). Using recurrent mass selection, intraspecific and interspecific crosses have been utilized to make new cultivars. Nowadays, there are more than twenty Fragaria species possessing multiple ploidy that change in size, color, flavor, shape, degree of fertility, season of ripening, susceptibility to disease and constitution of plant (Biswas et al. (2009) Sci Hortic. 122:409-416). With intraspecific crosses of the cultivated strawberry variety (F. x ananassa), improved agronomic traits are introduced into new cultivars. Pedigree selection, crossing of the best genotypes, and further selection are used for breeding for new strawberry cultivars because the strawberry cultivars are heterozygous and sensitive to inbreeding. Strawberry cultivars are then vegetatively propagated through runners (or stolons) as clones (Hancock, 1999). Also, a new strawberry cultivar can be developed through the induction of somaclonal variation from in vitro tissue culture and selection of suitable variants for further cultivation (Biswas et al., 2009). Somaclonal variation occurs by changes in chromosome number (polyploidy) or chromosome rearrangements by insertions, deletions, translocations, or mutation. The success of plant breeding by somaclonal variation depends on the selection of genetically stable somaclones.

Classic breeding methods can be included in the present disclosure to introduce one or more modified gene of the present disclosure into other plant varieties, or other close-related species that are compatible to be crossed with the transgenic plant of the present disclosure.

The present disclosure can be applied to other strawberry varieties to make them grow and produce fruits independent of environmental cues like photoperiod linked to day length and/or temperature (for example, vernalization).

TABLE 4
Sequence listing

| | | | Line or |
|---|---|---|---|
| SEQ | | | Figure |
| ID | | | described |
| NO: | Genus and Species | Description | herein |
| 1 | Fragaria sp. | 9 bp deletion in TFL1d1 | S4A-019 |
| 2 | Fragaria sp. | 1 bp insertion in TFL1d2 | |
| 3 | Fragaria sp. | 3 bp deletion in TFL1d1 | S4A-441 |
| 4 | Fragaria sp. | 1 bp insertion in TFL1d2 | |
| 5 | Fragaria sp. | 43 bp deletion in TFL1d1 | S4A-162 |
| 6 | Fragaria sp. | 4 bp deletion and 1 bp insertion in TFL1d2 | |
| 7 | Fragaria sp. | 3 bp deletion in TFL1d1 | S4A-198 |
| 8 | Fragaria sp. | 4 bp deletion in TFL1d2 | |
| 9 | Fragaria sp. | 3 bp deletion in TFL1d1 | S4A-353 |
| 10 | Fragaria sp. | 2 bp deletion in TFL1d2 | |
| 11 | Fragaria sp. | 3 bp deletion in TFL1d1 | S4A-046 |
| 12 | Fragaria sp. | 2 bp deletion in TFL1d2 | |
| 13 | Fragaria sp. | 3 bp deletion in TFL1d1 | S4A-448 |
| 14 | Fragaria sp. | 1 bp deletion in TFL1d2 | |
| 15 | Fragaria sp. | 9 bp deletion in TFL1d1 | S4A-194 |
| 16 | Fragaria sp. | 4 bp deletion in TFL1d2 | |
| 17 | Fragaria sp. | 3 bp deletion in TFL1d1 | S4A-260 |
| 18 | Fragaria sp. | 4 bp deletion in TFL1d2 | |
| 19 | Fragaria sp. | 12 bp deletion in TFL1d1 | S4A-018 |
| 20 | Fragaria sp. | 3 bp deletion in TFL1d2 | |
| 21 | Fragaria sp. | None; wild-type | |
| 22 | Fragaria sp. | 1 bp deletion in TFL1d1 | S4A-309 |
| 23 | Fragaria sp. | 4 bp deletion and 2 bp insertion in TFL1d2 | |
| 24 | Fragaria sp. | 10 bp deletion in TFL1d1 | S4A-051 |
| 25 | Fragaria sp. | 5 bp deletion in TFL1d2 | |
| 26 | Fragaria sp. | 1 bp deletion in TFL1d1 | S4A-138 |
| 27 | Fragaria sp. | 8 bp deletion in TFL1d2 | |
| 28 | Fragaria sp. | 25 bp deletion in TFL1d1 | S4A-039 |
| 29 | Fragaria sp. | 1 bp deletion in TFL1d2 | |
| 30 | Fragaria sp. | 18 bp deletion in TFL1d1 | S4A-176 |
| 31 | Fragaria sp. | 2 bp insertion in TFL1d2 | |
| 32 | Fragaria sp. | 89 bp deletion in TFL1d1 | S4A-186 |
| 33 | Fragaria sp. | 52 bp insertion in TFL1d2 | |
| 34 | Fragaria sp. | 4 bp deletion in TFL1d1 | S4A-233 |
| 35 | Fragaria sp. | 11 bp insertion in TFL1d2 | |
| 36 | Fragaria sp. | 14 bp deletion in TFL1d1 | S4A-236 |
| 37 | Fragaria sp. | 4 bp deletion in TFL1d2 | |
| 38 | Fragaria sp. | 20 bp deletion in TFL1d1 | S4A-029 |
| 39 | Fragaria sp. | 6 bp deletion and 1 bp insertion in TFL1d2 | |
| 40 | Fragaria sp. | 5 bp deletion in TFL1d1 | S4A-072 |
| 41 | Fragaria sp. | 4 bp deletion in TFL1d2 | |
| 42 | Fragaria sp. | deletion spanning D72-P74 of TFL1d1 | S4A-019 |
| 43 | Fragaria sp. | amino acid change from V73-G75 with an | |
| | | early stop codon in TFL1d2 | |
| 44 | Fragaria sp. | deletion of V73 in TFL1d1 | S4A-441 |
| 45 | Fragaria sp. | amino acid change from 73-G75 | |
| | | with an early stop codon in TFL1d2 | |
| 46 | Fragaria sp. | amino acid change from M68-P74 with an | S4A-162 |
| | | early stop codon in TFL1d1 | |
| 47 | Fragaria sp. | D72 change to E and deletion of V73 in | |
| | | TFL1d2 | |
| 48 | Fragaria sp. | deletion of D72 in TFL1d1 | S4A-198 |
| 49 | Fragaria sp. | amino acid change from V73-P79 with an | |
| | | early stop codon in TFL1d2 | |
| 50 | Fragaria sp. | deletion of D72 in TFL1d1 | S4A-353 |
| 51 | Fragaria sp. | amino acid change from D72-G75 with an | |
| | | early stop codon in TFL1d2 | |
| 52 | Fragaria sp. | deletion of D72 in TFL1d1 | S4A-046 |
| 53 | Fragaria sp. | amino acid change from V73-G75 with an | |
| | | early stop codon in TFL1d2 | |
| 54 | Fragaria sp. | deletion of V73 in TFL1d1 | S4A-448 |
| 55 | Fragaria sp. | amino acid change from D72-Y80 with an | |
| | | early stop codon in TFL1d2 | |
| 56 | Fragaria sp. | deletion of D72-P74 in TFL1d1 | S4A-194 |
| 57 | Fragaria sp. | amino acid change from V73-P79 with an | |
| | | early stop codon in TFL1d2 | |
| 58 | Fragaria sp. | deletion of V73 in TFL1d1 | S4A-260 |
| 59 | Fragaria sp. | amino acid change from D72-P79 with an | |
| | | early stop codon in TFL1d2 | |
| 60 | Fragaria sp. | deletion of D70-V73 in TFL1d1 | S4A-018 |
| 61 | Fragaria sp. | deletion of V73 in TFL1d2 | |
| 62 | Fragaria sp. | None; wild-type | |
| 63 | Fragaria sp. | Amino Acid Change from D72-Y80 with an | S4A-309 |
| | | early stop codon in TFL1d1 | |
| 64 | Fragaria sp. | Amino Acid Change from V73-G75 with an | |
| | | early stop codon in TFL1d2 | |
| 65 | Fragaria sp. | Amino Acid Change from D72-S77 with an | S4A-051 |
| | | early stop codon in TFL1d1 | |
| 66 | Fragaria sp. | Amino Acid Change from D72-P74 with an | |
| | | early stop codon in TFL1d2 | |
| 67 | Fragaria sp. | Amino Acid Change from D72-Y80 with an | S4A-138 |
| | | early stop codon in TFL1d1 | |
| 68 | Fragaria sp. | Amino Acid Change from D72-V73 with an | |
| | | early stop codon in TFL1d2 | |
| 69 | Fragaria sp. | early stop codon generation after D72 in | S4A-039 |
| | | TFL1d1 | |
| 70 | Fragaria sp. | Amino Acid Change from D72-Y80 with an | |
| | | early stop codon in TFL1d2 | |
| 71 | Fragaria sp. | deletion spanning V73-D78 in TFL1d1 | S4A-176 |
| 72 | Fragaria sp. | Amino Acid Change from D72-L81 with an | |
| | | early stop codon in TFL1d2 | |
| 73 | Fragaria sp. | deletion spanning V72-W87 in TFL1d1 | S4A-186 |
| 74 | Fragaria sp. | Amino Acid Change from V73-P79 with an | |
| | | early stop codon in TFL1d2 | |
| 75 | Fragaria sp. | Amino Acid Change from V73-P79 with an | S4A-233 |
| | | early stop codon in TFL1d1 | |
| 76 | Fragaria sp. | deletion spanning V73-R171 creating an early | |
| | | stop codon in TFL1d2 | |
| 77 | Fragaria sp. | early stop codon generation after P71 in | S4A-236 |
| | | TFL1d1 | |
| 78 | Fragaria sp. | Amino Acid Change from V73-P79 with an | |
| | | early stop codon in TFL1d2 | |
| 79 | Fragaria sp. | Amino Acid Change from D70-L81 with an | S4A-029 |
| | | early stop codon in TFL1d1 | |
| 80 | Fragaria sp. | Amino Acid Change from D72-P74 with an | |
| | | early stop codon in TFL1d2 | |
| 81 | Fragaria sp. | Amino Acid Change from D72-P74 with an | S4A-072 |
| | | early stop codon in TFL1d1 | |
| 82 | Fragaria sp. | Amino Acid Change from V73-P79 with an | |
| | | early stop codon in TFL1d2 | |
| 83 | Fragaria × ananassa | TFL1a-1 | FIG. 2 |
| 84 | Fragaria × ananassa | TFL1b_d | FIG. 2 |
| 85 | Fragaria × ananassa | TFL1c_e | FIG. 2 |
| 86 | Fragaria vesca | TFL2c-Fvb3 (+1) | FIG. 2 |
| 87 | Fragaria × ananassa | TFL2a_d_e (+1) | FIG. 2 |
| 88 | Fragaria × ananassa | TFL2b (+1) | FIG. 2 |
| 89 | Fragaria × ananassa | TFL2c (+1) | FIG. 2 |
| 90 | Fragaria vesca | TFL3f-Fvb7 (+1) | FIG. 2 |
| 91 | Fragaria × ananassa | TFL3a_b (+1) | FIG. 2 |
| 92 | Fragaria × ananassa | TFL3c (+1) | FIG. 2 |
| 93 | Fragaria × ananassa | TFL3d_f (+1) | FIG. 2 |
| 94 | Fragaria × ananassa | TFL3e (+1) | FIG. 2 |
| 95 | Fragaria × ananassa | TFL3g (+1) | FIG. 2 |
| 96 | Fragaria × ananassa | FT1a CDS (+1) | FIG. 2 |
| 97 | Fragaria × ananassa | FT1b CDS (+1) | FIG. 2 |
| 98 | Fragaria × ananassa | FT1c CDS (+1) | FIG. 2 |
| 99 | Fragaria × ananassa | FT1d CDS (+1) | FIG. 2 |
| 100 | Fragaria × ananassa | FT2a CDS (+1) | FIG. 2 |
| 101 | Fragaria × ananassa | FT2b CDS (+1) | FIG. 2 |
| 102 | Fragaria × ananassa | FT2c CDS (+1) | FIG. 2 |
| 103 | Fragaria vesca | FT-neg Fv3 CDS (+1) | FIG. 2 |
| 104 | Fragaria × ananassa | FT3e CDS (+1) | FIG. 2 |
| 105 | Fragaria vesca | MFT Fvb5 CDS (+1) | FIG. 2 |
| 106 | Fragaria × ananassa | Strawberry_TFL1d | FIG. 3A & 3B |
| 107 | Rubus idaeus | Red_Raspberry_TFL6g | FIG. 3A & 3B |
| 108 | Rubus occidentalis | Black_Raspberry_TFL6g | FIG. 3A & 3B |
| 109 | Rubus argutus | Sawtooth_Blackberry_TFL | FIG. 3A & 3B |
| 110 | Malus domestica | Apple_TFL14g | FIG. 3A & 3B |
| 111 | Malus domestica | Apple_TFL12g | FIG. 3A & 3B |
| 112 | Vaccinium corymbosum | Highbush_Blueberry_TFL21 | FIG. 3A & 3B |
| 113 | Vaccinium corymbosum | Highbush_Blueberry_TFL26 | FIG. 3A & 3B |
| 114 | Vaccinium corymbosum | Highbush_Blueberry_TFL29 | FIG. 3A & 3B |
| 115 | Vaccinium darrowii | Darrow's_Blueberry_TFL | FIG. 3A & 3B |
| 116 | Vaccinium corymbosum | Highbush_Blueberry_TFL33 | FIG. 3A & 3B |
| 117 | Fragaria × ananassa | Strawberry_FT | FIG. 3A & 3B |

Unless defined otherwise, all technical and scientific terms used herein have the same meaning as commonly understood by one of ordinary skill in the art to which this disclosure belongs. Generally, the nomenclature used herein, and the laboratory procedures in cell culture, molecular genetics, and nucleic acid chemistry and hybridization described herein, are those well-known and commonly employed in the art. Standard techniques are used for recombinant nucleic acid methods, polynucleotide synthesis, microbial culture, cell culture, tissue culture, transformation, transfection, transduction, analytical chemistry, organic synthetic chemistry, chemical syntheses, chemical analysis, and pharmaceutical formulation and delivery. Generally, enzymatic reactions and purification and/or isolation steps are performed according to the manufacturers' specifications. The techniques and procedures are generally performed according to conventional methodology disclosed, for example, in Molecular cloning a laboratory manual, 2ed., Cold Springs Harbor Laboratory Press, Cold Springs Harbor, N.Y. (1989), and Current protocols in molecular biology, John Wiley & Sons, Baltimore, Md. (1989).

All publications and patent applications mentioned in the specification are indicative of the level of those skilled in the art to which this disclosure pertains. All publications and patent applications are herein incorporated by reference to the same extent as if each individual publication or patent application was specifically and individually indicated to be incorporated by reference. Nothing herein is to be construed as an admission that the present disclosure is not entitled to antedate such publication by virtue of prior disclosure.

Many modifications and other embodiments of the disclosures set forth herein will come to mind to one skilled in the art to which these disclosures pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. Although specific terms are employed herein, they are used in a generic and descriptive sense only and not for purposes of limitation.

While the disclosure has been described in connection with specific embodiments thereof, it will be understood that it is capable of further modifications and this application is intended to cover any variations, uses, or adaptations of the disclosure following, in general, the principles of the disclosure and including such departures from the present disclosure as come within known or customary practice within the art to which the disclosure pertains and as may be applied to the essential features hereinbefore set forth and as follows in the scope of the appended claims.

All publications, patents and patent applications, including any drawings and appendices, and all nucleic acid sequences and polypeptide sequences identified by GenBank Accession numbers, herein are incorporated by reference to the same extent as if each individual publication or patent application was specifically and individually indicated to be incorporated by reference.

The foregoing description includes information that may be useful in understanding the present disclosure. It is not an admission that any of the information provided herein is prior art or relevant to the presently claimed disclosures, or that any publication specifically or implicitly referenced is prior art.

### EXAMPLES

### Example 1: Strawberry Plant Transformation

Plant transformation vectors were constructed using standard molecular biology techniques.

Inspire (BG-9.3142) strawberry plants were grown in Magenta boxes (Magenta™, Model GA-7, Millipore Sigma, St. Louis, MO) containing 75 mL of Fragaria x ananassa rooting (FaR) medium (half strength Murashige & Skoog Basal Medium with Vitamins, 15 g/L sucrose, 6 g/L agar, pH 5.7). Plants were cultured in a Percival growth chamber (Percival Scientific, Inc., Model CU-36L4, Perry, IA) with a 16-hour photoperiod of white fluorescent light (115 μmoles·m⁻²·s⁻¹) at 24° C. for 6 weeks. Leaves were cut and placed onto filter paper moistened with MS liquid medium (full strength Murashige & Skoog Basal Medium with Vitamins, 30 g/L sucrose, pH 5.7). The outer edges of each leaf were removed, and each leaf was cut into 3-5×3-5 mm rectangular explant sectors. Explants were then transferred onto pre-culture medium (TIA1 medium: full strength Murashige & Skoog Basal Medium with Vitamins, 30 g/L sucrose, 6 g/L agar, pH 5.7, 2 mg/L thidiazuron, 0.25 mg/L indole-3-acetic acid) with the abaxial surface in contact with the medium. Explants were cultured at 24° C. in the dark for 7 days (pre-culture).

After the pre-culture period, the explants were transferred to a sterile Petri plate with 15 to 25 mL of a transformed Agrobacterium tumefaciens culture containing 0.4% Tween-20. The Petri plates were incubated in the dark for 30 minutes at room temperature while shaking at 100 rpm on an orbital shaker. The Agrobacterium strain GV3101 had been transformed with the plasmid pSIM2797, and a culture grown to an optical density (OD₆₀₀) of 0.3. At the end of the incubation, the explants were blotted on sterile filter paper to remove excess Agrobacterium and transferred to co-culture medium (TIA1 medium). Explants were cultured at 24° C. in the dark for 48 hours (co-culture).

After the co-culture period, explants were transferred to shoot regeneration medium (TIA1) supplemented with 300 mg/L Timentin, 1 mL/L Plant Preservative Mixture (PPM™), and 150 mg/L kanamycin. These explants were cultured at 24° C. in the dark for 5 days. After 5 days, explants were transferred to shoot regeneration medium (TIA1) supplemented with 300 mg/L Timentin and 1 mL/L Plant Preservative Mixture (PPM™) and then cultured under white, fluorescent light (115 μmoles m⁻²·s⁻¹) with a 16-hour photoperiod for 23 days. Explants were then subcultured onto the same shoot regeneration medium and cultured for up to an additional 28 days.

As 4-5 mm shoot clumps formed, they were cut from the calli and transferred to Magenta boxes containing FaR medium supplemented with 300 mg/L Timentin and 1 mL/L PPM. Shoot clumps were cultured with a 16-hour photoperiod at 24° C. for 28 days. After 28 days, a single shoot per shoot clump was transferred into FaR medium supplemented with 300 mg/L Timentin and 1 mL/L PPM and were cultured for an additional 28 days before leaf samples were submitted for molecular characterization and event selection.

qPCR of gRNA6 and a control site was performed and a total of 5% of events returned positive for mutations in one or more alleles of TFL1. Mutated events were recollected by transformation and DNA was isolated via the CTAB method. Events were tested by ddPCR for the target site and an external reference (AGP). A number of events were confirmed to have at least one mutation in the TFL1 gene. Mutated events were designated S4A to encompass the variety ‘Inspire’ and the remontancy trait.

DNA was extracted from 100 selected S4A events (plus WT). To prepare for sequencing, a 440 bp amplicon was barcoded (IDT xGen UDI), products were checked on a 1% agarose gel, then pooled and sent to SeqMatic. Paired-end 250 nt reads were obtained using an Illumina MiSeq nano run. After demultiplexing, paired reads were merged, primers trimmed, and sequences clustered (CD-HIT) requiring 100% identity. The read counts in the various clusters were adjusted for PCR bias towards short amplicons, and unique allele dosage was calculated. The representative cluster reference sequences (CRS) were aligned to wildtype reference sequences and used to summarize the edits (shown in Tables 5a and 5b below). Seven alleles were identified and designated TFL1a, TFL1b1, TFL1b2, TFL1c, TFL1d1, TFL1d2, TFL1e.

TABLE 5a
Plant events having 3-6 edited alleles

| | | | | | | | | No. of |
|---|---|---|---|---|---|---|---|---|
| | | | | | | | | Edited |
| Event | TFL1a | TFL1b1 | TFL1b2 | TFL1c | TFL1d1 | TFL1d2 | TFL1e | Alleles |
| S4A-008 | 4D | 1D | 4D | 6D | 7I | — | WT | 6 |
| S4A-168 | WT | 2D | 10D | 39D | 5D | 1D | 3D | 6 |
| S4A-309 | WT | 10D | 2D | WT | 1D | 4D 2I | 4D 1I | 5 |
| S4A-049 | 2D | 68D | 1D | WT | — | 2D | 4D 1I | 6 |
| S4A-171 | — | 9D | 4D | WT | 38D 1I | 1D | 4D 1I | 6 |
| S4A-260 | 4D | 2D | — | WT | 3D | 4D | 1D | 6 |
| S4A-093 | 1D | 30D 4I | WT | WT | 4D | — | 2D | 5 |
| S4A-391 | 1D | 1D | 3D | WT | 2D | 1D 2I | WT | 5 |
| S4A-236 | 1D | 20D 20I | 1I | WT | 14D | 4D | 8D | 6 |
| S4A-018 | 89D | WT | 2D | WT | 12D | 3D | 3D | 5 |
| S4A-115 | 4D | 24D 5I | WT | 15D 15I | 31D | 1D | 1D | 6 |
| S4A-253 | 4D 1I | 2D | WT | 17D | 1D | 4D | 6D | 6 |
| S4A-180 | 8D | WT | WT | WT | 4D 1I | 2D 46I | WT | 3 |
| S4A-480 | 51D | WT | WT | 2D | 5D | 1D | 4D | 5 |
| S4A-186 | 27D | 3D | WT | — | 89D | 52I | 26D 20I | 6 |
| S4A-256 | WT | WT | WT | 11D | 30D 26I | 1D | 7D | 4 |
| S4A-227 | 1D | 28D | 9D | 1D | 1D | WT | 1I | 6 |
| S4A-390 | 24D | 2D | 1D | 3D | 14D | WT | — | 6 |
| S4A-336 | 3D | 22D 16I | 1D | 1D | 1D | WT | 11D 1I | 6 |
| S4A-098 | WT | 48D | 4D | WT | 4D 1I | WT | 18D | 4 |
| S4A-183 | WT | 1D | 9D | 87D 51I | 10D | WT | 9D | 5 |
| S4A-123 | WT | 16D | 22D 9I | WT | 3D 2I | WT | 5D | 4 |
| S4A-378 | 1I | 9D | 1D | 1D | 21D 20I | WT | — | 6 |
| S4A-119 | 1D | 5D | 3D 1I | 1D | WT | 4D | WT | 5 |
| S4A-130 | 1D | 41D 8I | 2D | WT | 4D | WT | 1D | 5 |
| S4A-293 | WT | 22D | WT | 16D | WT | WT | 25D | 3 |
| S4A-278 | 7D | 16D | 4D | 8D | WT | WT | 1D | 5 |
| S4A-334 | 48D | 4D | 4D | 1D | WT | 4D | WT | 5 |

D = deletion of the number of nucleotides indicated by the preceding number. I = insertion of the number of nucleotides indicated by the preceding number. WT = wild type. A dash indicates deletion present but not quantified.

TABLE 5b
Plant events having 7 edited alleles

| | | | | | | | | No. of |
|---|---|---|---|---|---|---|---|---|
| | | | | | | | | Edited |
| Event | TFL1a | TFL1b1 | TFL1b2 | TFL1c | TFL1d1 | TFL1d2 | TFL1e | Alleles |
| S4A-027 | — | 89D | 3D | 14D | 25D | 1D | 16D | 7 |
| S4A-126 | 9D | 5D | 1D | 29D | 5D | — | 5D | 7 |
| S4A-110 | 3D 1I | 2D | 4D | 87D | 22D 20I | 11D 33I | — | 7 |
| S4A-029 | 1D | 21D | 22I | 1D | 20I | 6D 1I | 2D | 7 |
| S4A-039 | — | 89D | 3D | 14D | 25D | 1D | 16D | 7 |
| S4A-019 | 1D | 3D | — | 2D | 9D | 1I | 3D 1I | 7 |
| S4A-347 | 31D | 33D | 4D | — | — | — | — | 7 |
| S4A-425 | 21D | 9D | 1D | — | 4D | 4D | 17D | 7 |
| S4A-426 | 31D | 4D | 1D | 4D | 26D | 31D | 1D | 7 |
| S4A-446 | 1D | 5D | 2D | 1D | 26D | 2D | 1I | 7 |
| S4A-051 | 16D | 4D | 4D | 10D | 10D | 5D | 19D | 7 |
| S4A-072 | 2D | 4D | 4D | 20D 13I | 5D | 4D | 1D | 7 |
| S4A-073 | 4D | 3D | 16I | 1D | 4D | — | 1D | 7 |
| S4A-134 | 4D | 4D | 1D | — | 1I | 5D | 9D | 7 |
| S4A-149 | 9D | 15D | 1D | 10D | 7D | 1D | 1D | 7 |
| S4A-165 | 16D | 16D | 10D | 1D | 10D | — | 1D | 7 |
| S4A-198 | 31D | 23D 3I | 2D | 16D | 3D | 4D | 2D | 7 |
| S4A-353 | 1D | 42D | 1D | 4D | 3D | 2D | 1D | 7 |
| S4A-046 | 1D | 9D | 4D | 4D 1I | 3D | 2D | 87D | 7 |
| S4A-048 | 1D | 1D | 3D | 6D | 12D | 6D | 4D | 7 |
| S4A-140 | 4D | 1D | 1D | 10D | 4D | 1D | 1D | 7 |
| S4A-154 | 9D | 17D | 5D | 1I | 4D | 4D 1I | 4D | 7 |
| S4A-167 | 12D 20I | 30D 15I | 15D | 28D | 1D | 23I | 209D | 7 |
| S4A-176 | 3D 4I | 4D | 27I | 4D 1I | 18D | 1D 3I | 26D | 7 |
| S4A-196 | 4D 2I | 5D | 1D | — | 18D | — | 5D | 7 |
| S4A-307 | — | 30D 4I | 4D | 4D | 10D | 3D | 3D | 7 |
| S4A-437 | 9D | 8D | 25D | 1D | 32D 11I | 10D | — | 7 |
| S4A-443 | 2D | 9D | 5D | 63D | 44D | 3D | 8D | 7 |
| S4A-448 | 1D | 3D | 5D | 1I | 3D | 1D | 3D 2I | 7 |
| S4A-170 | 2D | 1D | 1D | 2D | 8D | 3D | 10D | 7 |
| S4A-194 | 2D | 11 | 4D | 2D | 9D | 4D | 24D | 7 |
| S4A-276 | — | 10D | — | 17D 1I | 19D | 1D | 5D | 7 |
| S4A-382 | 1D 1I | 15D | 3D | 15D 12I | 4D | 4D 1I | 2D | 7 |
| S4A-441 | 1D | 3D | 4D | 2D | 3D | 1I | 13D | 7 |
| S4A-444 | 1D | 2D | 2D | 1D | 4D2I | 1D | 14D | 7 |
| S4A-109 | 1D | 2D | 1D | 16D | 2D 1I | 1D | 28D 28I | 7 |
| S4A-137 | 45D 5I | 31D 3I | 13D 7I | — | 28D 18I | — | — | 7 |
| S4A-214 | — | 25D | 25D | 1D | 18D | 2D | 56D | 7 |
| S4A-277 | 20D | 21D 12I | 22D 14I | 66D | 16D | 1D | 26D | 7 |
| S4A-024 | 4D | 1D | — | 9D | 1D | 1D | 14D | 7 |
| S4A-047 | 1D | 4D | 3D | 3D | 1D | — | 26D | 7 |
| S4A-065 | 1D | 1D | 4D | 16D | 4D | 50I | 11D | 7 |
| S4A-076 | 31D 6I | 17D | 4D | 1I | 10D | 4D | 1D | 7 |
| S4A-162 | 1D | 1D | 1D | 3D 1I | 43D | 4D 1I | 8D | 7 |
| S4A-217 | 25D | 21D 11I | 4D 1I | 18D 13I | 4D | 4D | 1D | 7 |
| S4A-358 | 1D | 1D | 8I | 4D | 7I | 4D | 1D | 7 |
| S4A-400 | 4D | 1D II | — | 1D | 4D | 1D | 2D | 7 |
| S4A-402 | 4D | 5D | 3D | 14D | 4D | 1D | 1D | 7 |
| S4A-458 | 2D | 1D | 2D | 6D | 1D | 1D | 1D | 7 |
| S4A-020 | 1D | 19D | 10 D | 4D | 26D | 3D | 1D | 7 |
| S4A-201 | 9D | 20D | 3D | 1D | 1I | 1D | 24D | 7 |
| S4A-209 | 1D | 4D | 1D | 9D | 1D | 1D | 1D | 7 |
| S4A-233 | 1D | 28D | 2D | 3D | 4D | 11I | 14D | 7 |
| S4A-411 | 4D 1I | 4D | 1D | 1D | 15D | 4D | 14D | 7 |
| S4A-237 | 6D | 38D 2I | 1D | 4D | 9D | 5D | 1D | 7 |
| S4A-210 | 3D | 18D 17I | 1D | 4D 1I | 1D | 4D | 2D | 7 |
| S4A-246 | — | 1D | 1I | 8D | 8D | 6D 6I | 1D | 7 |
| S4A-274 | — | 1I | — | 2D | 26D | 10D | 1D | 7 |
| S4A-031 | — | 1D | 4d 1I | 4D | 1D | 28I | 2D | 7 |
| S4A-479 | 8D | 10D | 4D | 4D | 11D | 4D 1I | 14D | 7 |
| S4A-138 | 10D | 4D | 3D | 79D | 1D | 8D | — | 7 |
| S4A-234 | 7D | 4D | 4D 1I | 8D | 1I | 31D 17I | 1D | 7 |
| S4A-108 | 4D | 20D | 3D | 10D | 5D | 4D 1I | 1D | 7 |
| S4A-163 | 4D 1I | 5D | 9D | 11D | 9D | — | 1D | 7 |
| S4A-447 | 38D | 72D | 28D | — | 22D | 4D | 2D | 7 |
| S4A-445 | 4D 1I | 26D | 1D | 36D 16I | 6D | 2D | 3D | 7 |

### Example 2: Greenhouse Assessment—Weeks to Anthesis After Planting

Strawberry plants regenerated from transformation and scored as positive by the ddPCR assay for mutations were transferred to soil-less growing medium in 6-inch pots and grown in a greenhouse. The greenhouse ambient temperatures were maintained at 68-78° F. during the day and 62-72° F. during the night. Light was provided from 6 AM to 10 PM, which constitutes a long day photoperiod. Natural light was the main light source, with high-pressure sodium fixtures providing supplemental lighting as needed. Plants were hand watered as necessary and fertilized every two weeks.

Plants with at least one open flower (OF) were documented at weekly intervals (Table 6a-6e and FIG. 5). Assessments concluded at 33 weeks after planting (WAP). The wild type controls consisted of sixteen independent events. Control and S4A events included three replicates. Tables 6a-6d show edits for TFL1d1 and TFL1d2 alleles only; a complete description of edits for each allele in each event are given in Tables 5a and 5b above.

TABLE 6a
Weeks to anthesis after planting for plants
having both TFL1d1 and TFL1d2 alleles edited

| | | | | | Total # |
|---|---|---|---|---|---|
| | Weeks Earlier | Weeks After | TFL1d1 | TFL1d2 | of Edited |
| Event | than Wildtype | Planting | Edit | Edit | Alleles |
| WT | n/a | 27.5 | n/a | n/a | 0 |
| S4A-008 | 21.2 | 6.3 | 7I | — | 6 |
| S4A-168 | 21.2 | 6.3 | 5D | 1D | 6 |
| S4A-309 | 19.8 | 7.7 | 1D | 4D 2I | 5 |
| S4A-049 | 19.8 | 7.7 | — | 2D | 6 |
| S4A-171 | 18.8 | 8.7 | 38D 1I | 1D | 6 |
| S4A-260 | 18.5 | 9.0 | 3D | 4D | 6 |
| S4A-093 | 18.2 | 9.3 | 4D | — | 5 |
| S4A-391 | 18.2 | 9.3 | 2D | 1D 2I | 5 |
| S4A-236 | 18.2 | 9.3 | 14D | 4D | 6 |
| S4A-018 | 17.8 | 9.7 | 12D | 3D | 5 |
| S4A-115 | 17.8 | 9.7 | 31D | 1D | 6 |
| S4A-253 | 17.5 | 10.0 | 1D | 4D | 6 |
| S4A-180 | 17.2 | 10.3 | 4D1I | 2D 46I | 3 |
| S4A-480 | 17.2 | 10.3 | 5D | 1D | 5 |
| S4A-186 | 16.8 | 10.7 | 89D | 52I | 6 |
| S4A-256 | 16.2 | 11.3 | 30D 26I | 1D | 4 |
| Avg. | 18.4 | 9.1 | | | |

TABLE 6b
Weeks to anthesis after planting for plants
having all 7 TFL alleles edited

| | | | | | Total # |
|---|---|---|---|---|---|
| | Weeks Earlier | Weeks After | TFL1d1 | TFL1d2 | of Edited |
| Event | than Wildtype | Planting | Edit | Edit | Alleles |
| WT | n/a | 27.5 | n/a | n/a | 0 |
| S4A-027 | 21.8 | 5.7 | 25D | 1D | 7 |
| S4A-126 | 21.8 | 5.7 | 5D | — | 7 |
| S4A-110 | 21.5 | 6.0 | 22D 20I | 11D 33I | 7 |
| S4A-029 | 21.2 | 6.3 | 20I | 6D 1I | 7 |
| S4A-039 | 21.2 | 6.3 | 25D | 1D | 7 |
| S4A-019 | 20.8 | 6.7 | 9D | 1I | 7 |
| S4A-347 | 20.5 | 7.0 | — | — | 7 |
| S4A-425 | 20.5 | 7.0 | 4D | 4D | 7 |
| S4A-426 | 20.5 | 7.0 | 26D | 31D | 7 |
| S4A-446 | 20.5 | 7.0 | 26D | 2D | 7 |
| S4A-051 | 20.2 | 7.3 | 10D | 5D | 7 |
| S4A-072 | 20.2 | 7.3 | 5D | 4D | 7 |
| S4A-073 | 20.2 | 7.3 | 4D | — | 7 |
| S4A-134 | 20.2 | 7.3 | 1I | 5D | 7 |
| S4A-149 | 20.2 | 7.3 | 7D | 1D | 7 |
| S4A-165 | 20.2 | 7.3 | 10D | — | 7 |
| S4A-198 | 20.2 | 7.3 | 3D | 4D | 7 |
| S4A-353 | 20.2 | 7.3 | 3D | 2D | 7 |
| S4A-046 | 19.8 | 7.7 | 3D | 2D | 7 |
| S4A-048 | 19.8 | 7.7 | 12D | 6D | 7 |
| S4A-140 | 19.8 | 7.7 | 4D | 1D | 7 |
| S4A-154 | 19.8 | 7.7 | 4D | 4D 1I | 7 |
| S4A-167 | 19.8 | 7.7 | 1D | 23I | 7 |
| S4A-176 | 19.8 | 7.7 | 18D | 1D 3I | 7 |
| S4A-196 | 19.8 | 7.7 | 18D | — | 7 |
| S4A-307 | 19.8 | 7.7 | 10D | 3D | 7 |
| S4A-437 | 19.8 | 7.7 | 32D 11I | 10D | 7 |
| S4A-443 | 19.8 | 7.7 | 44D | 3D | 7 |
| S4A-448 | 19.8 | 7.7 | 3D | 1D | 7 |
| S4A-170 | 19.5 | 8.0 | 8D | 3D | 7 |
| S4A-194 | 19.5 | 8.0 | 9D | 4D | 7 |
| S4A-276 | 19.5 | 8.0 | 19D | 1D | 7 |
| S4A-382 | 19.5 | 8.0 | 4D | 4D 1I | 7 |
| S4A-441 | 19.5 | 8.0 | 3D | 1I | 7 |
| S4A-444 | 19.5 | 8.0 | 4D2I | 1D | 7 |
| S4A-109 | 19.2 | 8.3 | 2D 1I | 1D | 7 |
| S4A-137 | 19.2 | 8.3 | 28D 18I | — | 7 |
| S4A-214 | 19.2 | 8.3 | 18D | 2D | 7 |
| S4A-277 | 19.2 | 8.3 | 16D | 1D | 7 |
| S4A-024 | 18.8 | 8.7 | 1D | 1D | 7 |
| S4A-047 | 18.8 | 8.7 | 1D | — | 7 |
| S4A-065 | 18.8 | 8.7 | 4D | 50I | 7 |
| S4A-076 | 18.8 | 8.7 | 10D | 4D | 7 |
| S4A-162 | 18.8 | 8.7 | 43D | 4D 1I | 7 |
| S4A-217 | 18.8 | 8.7 | 4D | 4D | 7 |
| S4A-358 | 18.8 | 8.7 | 7I | 4D | 7 |
| S4A-400 | 18.8 | 8.7 | 4D | 1D | 7 |
| S4A-402 | 18.8 | 8.7 | 4D | 1D | 7 |
| S4A-458 | 18.8 | 8.7 | 1D | 1D | 7 |
| S4A-020 | 18.5 | 9.0 | 26D | 3D | 7 |
| S4A-201 | 18.5 | 9.0 | 1I | 1D | 7 |
| S4A-209 | 18.5 | 9.0 | 1D | 1D | 7 |
| S4A-233 | 18.5 | 9.0 | 4D | 11I | 7 |
| S4A-411 | 18.5 | 9.0 | 15D | 4D | 7 |
| S4A-237 | 18.2 | 9.3 | 9D | 5D | 7 |
| S4A-210 | 17.8 | 9.7 | 1D | 4D | 7 |
| S4A-246 | 17.8 | 9.7 | 8D | 6D 6I | 7 |
| S4A-274 | 17.8 | 9.7 | 26D | 10D | 7 |
| S4A-031 | 17.5 | 10.0 | 1D | 28I | 7 |
| S4A-479 | 17.5 | 10.0 | 11D | 4D 1I | 7 |
| S4A-138 | 17.2 | 10.3 | 1D | 8D | 7 |
| S4A-234 | 17.2 | 10.3 | 1I | 31D 17I | 7 |
| S4A-108 | 16.5 | 11.0 | 5D | 4D 1I | 7 |
| S4A-163 | 16.5 | 11.0 | 9D | — | 7 |
| S4A-447 | 16.2 | 11.3 | 22D | 4D | 7 |
| S4A-445 | 14.8 | 12.7 | 6D | 2D | 7 |
| Avg. | 19.2 | 8.3 | | | |

TABLE 6c
Average weeks to anthesis after planting for plants having only one
of TFL1d1 and TFL1d2 alleles edited (noted as diamonds in FIG. 5)

| | | | | | Total # |
|---|---|---|---|---|---|
| | Weeks Earlier | Weeks After | TFL1d1 | TFL1d2 | of Edited |
| Event | than Wildtype | Planting | Edit | Edit | Alleles |
| S4A-227 | 11.5 | 16.0 | 1D | WT | 6 |
| S4A-390 | 8.8 | 18.7 | 14D | WT | 6 |
| S4A-336 | 6.5 | 21.0 | 1D | WT | 6 |
| S4A-098 | 5.5 | 22.0 | 4D 1I | WT | 4 |
| S4A-183 | 4.5 | 23.0 | 10D | WT | 5 |
| S4A-123 | 3.5 | 24.0 | 3D 2I | WT | 4 |
| S4A-378 | 3.5 | 24.0 | 21D 20I | WT | 6 |
| S4A-119 | 1.0 | 26.5 | WT | 4D | 5 |
| S4A-130 | −3.2 | 30.7 | 4D | WT | 5 |
| S4A-293 | −4.8 | 32.3 | WT | WT | 3 |
| S4A-278 | −5.5 | 33.0 | WT | WT | 5 |
| S4A-334 | −5.5 | 33.0 | WT | 4D | 5 |
| Avg. | 2.15 | 25.35 | | | |

TABLE 6d
Summary of greenhouse flowering data shown in Tables 6a-6c

| | | Average | | | |
|---|---|---|---|---|---|
| No. of | | Weeks | Average | | |
| Alleles | # of | Earlier than | Weeks to | Earliest | Latest |
| Edited | Events | WT | Anthesis | Week | Week |
| 7 | 66 | 19.2 ± 1.3 | 8.3 ± 1.3 | 5.7 | 12.7 |
| 6 | 13 | 15.4 ± 5.8 | 12.1 ± 5.8 | 6.3 | 24 |
| 5 | 10 | 8.25 ± 10.9 | 19.3 ± 10.9 | 7.7 | 33 |
| 4 | 3 | 8.4 ± 6.8 | 19.1 ± 6.8 | 11.3 | 24 |
| 3 | 2* | 6.2 ± 15.6 | 21.3 ± 15.6 | 10.3 | 32.3 |
| 0-empty | 3 | n/a | 32.4 ± 0.5 | 32 | 33 |
| vector | | | | | |
| Wild Type | 16 | n/a | 27.5 ± 2.7 | 21 | 31 |

Each event comprised three biological replicates
*one line comprised both TFL1d1 and TFL1d2 edits and flowered 17.2 weeks earlier than wildtype, while the other line had wildtype TFL1d1 and TFL1d2 alleles and flowered 4.8 weeks after wildtype

TABLE 6e
Summary of greenhouse flowering data shown in Tables
6a-6b (plants having both TFL1d1 and TFL1d2 edited)

| No. of | | Average | Average | | |
|---|---|---|---|---|---|
| Alleles | # of | Weeks Earlier | Weeks to | Earliest | Latest |
| Edited | Events | than WT | Anthesis | Week | Week |
| 7 | 66 | 19.2 ± 1.3 | 8.3 ± 1.3 | 5.7 | 12.7 |
| 6 | 9 | 18.7 ± 1.5 | 8.6 ± 1.5 | 6.3 | 10.7 |
| 5 | 5 | 18.2 ± 1.0 | 9.3 ± 1.0 | 7.7 | 10.3 |
| 4 | 1* | 16.2 | 11.3 | 11.3 | 11.3 |
| 3 | [symbol] | 17.2 | 10.3 | 10.3 | 10.3 |
| 0-empty | 3 | n/a | 32.4 ± 0.5 | 32 | 33 |
| vector | | | | | |
| Wild Type | 16 | n/a | 27.5 ± 2.7 | 21 | 31 |

Each event comprised three biological replicates
*line S4A-256 having mutations in TFL1c and TFL1e in addition to TFL1d1 and TFL1d2 line S4A-180 having mutations in TFL1a in addition to TFL1d1 and TFL1d2

As shown in Tables 6a-6e and FIG. 5, there is an inverse trend for the number of TFL alleles edited and weeks to flower, wherein the greater number of TFL alleles edited correlates with earlier weeks to flower compared to wildtype. When plants having at least one wild-type TFL1d allele are removed from the summary data shown in Table 6d above, the average weeks to flower jumps from 12.1 to 8.6, or, from 15.4 weeks earlier than wild-type to 18.7 weeks earlier than wild-type (Table 6e, comparing those plants having 6 edited alleles in Tables 6d and 6e).

Similarly, comparing those plants having 5 edited alleles but excluding those wherein both TFL1d alleles were not edited, the average weeks to flower jumps from 19.3 to 9.3, or, from 8.25 weeks earlier than wild-type to 18.2 weeks earlier than wild-type.

For plants having four TFL alleles edited, line S4A-256, which comprised edits in both TFL1d1 and TFL1d2, flowered 16.2 weeks earlier than wild-type. The other lines having 4 edited alleles that did not comprise edits to both TFL1d1 and TFL1d2 alleles only flowered 5.5 and 3.5 weeks earlier than wild-type.

Lastly, line S4A-180, which comprised edits in TFL1d1, TFL1d2, and TFL1a flowered 17.2 weeks earlier than wild-type, whereas the other line having three edited alleles (S4A-293), comprising edits to TFL1b1, TFL1c, and TFL1e, flowered 4.8 weeks after wild-type plants.

Thus, plants having genetic edits to both TFL1d1 and TFL1d2 alleles flowered earlier than those having only one edited TFL1d allele, or those wherein other TFL alleles were targeted. On average, plants comprising edits to both TFL1d1 and TFL1d2 alleles flowered 18.4 weeks earlier than wildtype (9.1 weeks after planting compared to 27.5 weeks after planting). When all TFL alleles were edited, plants flowered on average 19.2 weeks earlier than wildtype (8.3 weeks after planting compared to 27.5 weeks after planting).

### Example 3: Field Trials—Flowering

Fall planted 2021-22 fields consisted of plug plants grown in a greenhouse in Idaho. Twelve rooted plug plants per event were hand planted into raised beds with plastic mulch in Oxnard, CA on Oct. 11, 2021, and Salinas, CA on Nov. 30, 2021. No chilling was provided prior to planting. After the planting in Oxnard, stress and minor dieback was noted so the plug plants destined for Salinas were acclimated in pots of soilless media for 2 weeks near the field site before transplanting to the field. Plants were maintained according to usual grower practices.

Fall planted 2022-23 fields consisted of bareroot plants produced in a high elevation nursery located at Macdoel, CA. Following commercial standards, plants were harvested after 265 chill hours for Oxnard and 447 chill hours for Salinas. Two reps of twenty plants were hand planted into raised beds with plastic mulch in Oxnard, CA on Oct. 11, 2022, and Salinas, CA on Nov. 11, 2022. Plants were maintained according to usual grower practices.

Summer planted 2023 field consisted of bareroot plants produced in a low elevation nursery located at Manteca, CA. Plants were harvested and placed into cold storage (32 F) for approximately 6 weeks. Two reps of twenty plants were hand planted into raised beds with plastic mulch in Oxnard, CA on Jul. 13, 2023. Plants were maintained according to the usual grower practices.

Plants with at least one open flower were documented at various intervals or weeks after planting (WAP) over the growing season. The number of viable plants per plot were documented to find the percent of plants per plot that had reached anthesis.

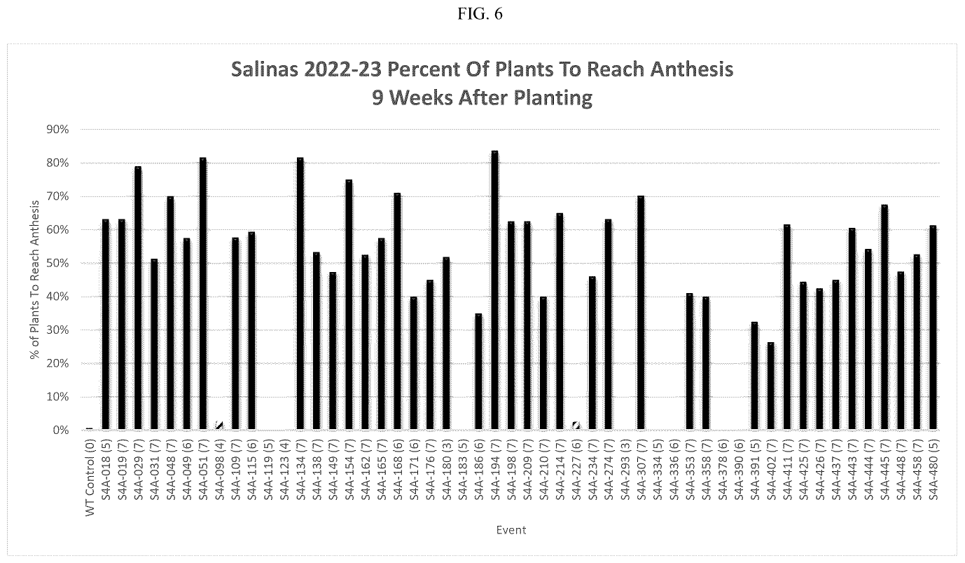

A subset of the data shown in Table 7b is graphed in FIG. 6. Plant events wherein at least one wildtype TFL1d1 and/or TFL1d2 allele remain are noted with an asterisk.

TABLE 7a
Field flowering data collected in Salinas, California in 2021-2022

Salinas, California 2021-2022

| Event (# edited | 1 Week | 5 Weeks | 11 weeks |
|---|---|---|---|
| alleles) | % flowering | % flowering | % flowering |
| WT Control (0) | 7% | 24% | 87% |
| S4A-008 (6) | 13% | 25% | 75% |
| S4A-018 (5) | 0% | 67% | 100% |
| S4A-019 (7) | 0% | 67% | 100% |
| S4A-020 (7) | 14% | 50% | 100% |
| S4A-024 (7) | 50% | 67% | 100% |
| S4A-029 (7) | 42% | 67% | 100% |
| S4A-031 (7) | 27% | 64% | 91% |
| S4A-046 (7) | 13% | 63% | 100% |
| S4A-047 (7) | 22% | 44% | 89% |
| S4A-048 (7) | 40% | 40% | 90% |
| S4A-049 (6) | 13% | 63% | 88% |
| S4A-051 (7) | 33% | 58% | 92% |
| S4A-073 (7) | 0% | 60% | 100% |
| S4A-076 (7) | 0% | 25% | 75% |
| S4A-098 (4)* | 29% | 57% | 86% |
| S4A-108 (7) | 0% | 14% | 71% |
| S4A-109 (7) | 13% | 38% | 100% |
| S4A-115 (6) | 0% | 25% | 86% |
| S4A-119 (5)* | 0% | 0% | 100% |
| S4A-123 (4)* | 33% | 75% | 100% |
| S4A-126 (7) | 50% | 83% | 100% |
| S4A-130 (5)* | 8% | 50% | 91% |
| S4A-134 (7) | 20% | 40% | 100% |
| S4A-137 (7) | 25% | 42% | 100% |
| S4A-138 (7) | 17% | 75% | 92% |
| S4A-149 (7) | 25% | 42% | 92% |
| S4A-163 (7) | 42% | 58% | 100% |
| S4A-165 (7) | 20% | 80% | 100% |
| S4A-167 (7) | 100% | 80% | 100% |
| S4A-168 (6) | 40% | 50% | 100% |
| S4A-170 (7) | 25% | 50% | 92% |
| S4A-171 (6) | 17% | 75% | 100% |
| S4A-176 (7) | 40% | 40% | 90% |
| S4A-180 (3) | 50% | 83% | 83% |
| S4A-186 (6) | 67% | 83% | 100% |
| S4A-194 (7) | 42% | 58% | 100% |
| S4A-196 (7) | 10% | 40% | 90% |
| S4A-201 (7) | 55% | 73% | 100% |
| S4A-210 (7) | 25% | 42% | 100% |
| S4A-214 (7) | 50% | 50% | 100% |
| S4A-227 (6)* | 33% | 50% | 100% |
| S4A-233 (7) | 30% | 60% | 80% |
| S4A-234 (7) | 36% | 64% | 100% |
| S4A-236 (6) | 50% | 75% | 100% |
| S4A-253 (6) | 29% | 29% | 100% |
| S4A-274 (7) | 56% | 78% | 100% |
| S4A-293 (3)* | 25% | 25% | 100% |
| S4A-307 (7) | 27% | 36% | 100% |
| S4A-309 (5) | 17% | 58% | 91% |
| S4A-336 (6)* | 33% | 50% | 100% |
| S4A-353 (7) | 33% | 67% | 100% |
| S4A-358 (7) | 13% | 25% | 88% |
| S4A-400 (7) | 50% | 50% | 100% |
| S4A-402 (7) | 25% | 67% | 100% |
| S4A-411 (7) | 50% | 75% | 100% |
| S4A-425 (7) | 50% | 50% | 80% |
| S4A-426 (7) | 92% | 100% | 100% |
| S4A-437 (7) | 33% | 50% | 100% |
| S4A-444 (7) | 67% | 67% | 100% |
| S4A-445 (7) | 50% | 58% | 100% |
| S4A-448 (7) | 64% | 55% | 100% |
| S4A-479 (7) | 8% | 17% | 92% |

TABLE 7b
Field flowering data collected in Salinas, California in 2022-2023

Salinas, California 2022-2023

| Event (# edited | 5 Weeks | 9 Weeks | 14 weeks |
|---|---|---|---|
| alleles) | % flowering | % flowering | % flowering |
| WT Control (0) | 0% | 1% | 37% |
| S4A-018 (5) | 18% | 63% | 82% |
| S4A-019 (7) | 28% | 63% | 74% |
| S4A-029 (7) | 42% | 79% | 95% |
| S4A-031 (7) | 27% | 51% | 70% |
| S4A-048 (7) | 25% | 70% | 83% |
| S4A-049 (6) | 33% | 58% | 80% |
| S4A-051 (7) | 39% | 82% | 92% |
| S4A-098 (4)* | 0% | 3% | 49% |
| S4A-109 (7) | 32% | 58% | 94% |
| S4A-115 (6) | 20% | 59% | 95% |
| S4A-119 (5)* | 0% | 0% | 61% |
| S4A-123 (4)* | 0% | 0% | 43% |
| S4A-134 (7) | 26% | 82% | 95% |
| S4A-138 (7) | 30% | 53% | 79% |
| S4A-149 (7) | 16% | 47% | 95% |
| S4A-154 (7) | 23% | 75% | 88% |
| S4A-162 (7) | 20% | 53% | 70% |
| S4A-165 (7) | 33% | 58% | 85% |
| S4A-168 (6) | 34% | 71% | 97% |
| S4A-171 (6) | 10% | 40% | 75% |
| S4A-176 (7) | 15% | 45% | 90% |
| S4A-180 (3) | 26% | 52% | 97% |
| S4A-183 (5)* | 0% | 0% | 28% |
| S4A-186 (6) | 18% | 35% | 78% |
| S4A-194 (7) | 54% | 84% | 95% |
| S4A-198 (7) | 35% | 63% | 70% |
| S4A-209 (7) | 23% | 63% | 98% |
| S4A-210 (7) | 10% | 40% | 80% |
| S4A-214 (7) | 18% | 65% | 93% |
| S4A-227 (6)* | 0% | 3% | 70% |
| S4A-234 (7) | 13% | 46% | 90% |
| S4A-274 (7) | 29% | 63% | 87% |
| S4A-293 (3)* | 0% | 0% | 28% |
| S4A-307 (7) | 46% | 70% | 94% |
| S4A-334 (5)* | 0% | 0% | 50% |
| S4A-336 (6)* | 0% | 0% | 48% |
| S4A-353 (7) | 21% | 41% | 74% |
| S4A-358 (7) | 20% | 40% | 70% |
| S4A-378 (6)* | 0% | 0% | 26% |
| S4A-390 (6)* | 0% | 0% | 20% |
| S4A-391 (5) | 15% | 33% | 73% |
| S4A-402 (7) | 8% | 26% | 63% |
| S4A-411 (7) | 23% | 62% | 83% |
| S4A-425 (7) | 18% | 44% | 90% |
| S4A-426 (7) | 18% | 43% | 83% |
| S4A-437 (7) | 18% | 45% | 83% |
| S4A-443 (7) | 32% | 61% | 89% |
| S4A-444 (7) | 19% | 54% | 97% |
| S4A-445 (7) | 18% | 68% | 89% |
| S4A-448 (7) | 15% | 48% | 83% |
| S4A-458 (7) | 13% | 53% | 89% |
| S4A-480 (5) | 28% | 61% | 87% |

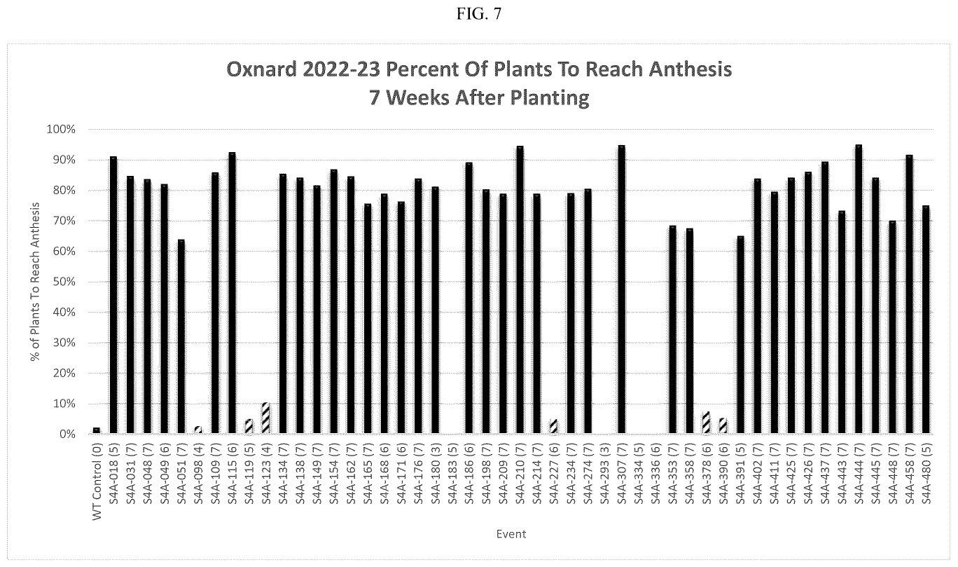

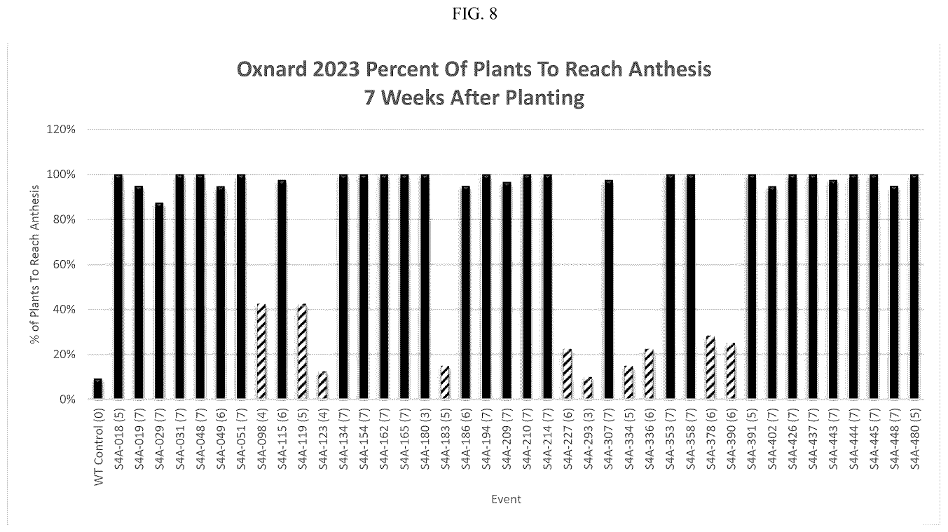

A subset of the data shown in Table 8b and 8c are graphed in FIGS. 7 and 8. Plant events wherein at least one wildtype TFL1d1 and/or TFL1d2 allele remain are noted with an asterisk.

TABLE 8a
Field flowering data collected in Oxnard, California in 2021-2022

Oxnard, California 2021-2022

| Event (# edited | 6 Weeks | 10 Weeks | 14 weeks |
|---|---|---|---|
| alleles) | % flowering | % flowering | % flowering |
| WT Control (0) | 32% | 79% | 99% |
| S4A-008 (6) | 67% | 67% | 83% |
| S4A-018 (5) | 45% | 100% | 100% |
| S4A-019 (7) | 82% | 100% | 100% |
| S4A-020 (7) | 73% | 91% | 100% |
| S4A-024 (7) | 92% | 100% | 100% |
| S4A-027 (7) | 86% | 100% | 100% |
| S4A-029 (7) | 100% | 100% | 92% |
| S4A-031 (7) | 92% | 100% | 100% |
| S4A-046 (7) | 63% | 88% | 100% |
| S4A-047 (7) | 83% | 100% | 100% |
| S4A-048 (7) | 73% | 91% | 91% |
| S4A-049 (6) | 91% | 100% | 100% |
| S4A-051 (7) | 80% | 100% | 100% |
| S4A-065 (7) | 100% | 100% | 100% |
| S4A-072 (7) | 56% | 100% | 100% |
| S4A-073 (7) | 89% | 100% | 100% |
| S4A-076 (7) | 91% | 100% | 100% |
| S4A-098 (4)* | 55% | 91% | 100% |
| S4A-108 (7) | 91% | 100% | 100% |
| S4A-109 (7) | 75% | 100% | 100% |
| S4A-110 (7) | 100% | 100% | 100% |
| S4A-115 (6) | 100% | 100% | 100% |
| S4A-123 (4)* | 55% | 100% | 100% |
| S4A-126 (7) | 88% | 100% | 100% |
| S4A-130 (5)* | 45% | 100% | 100% |
| S4A-134 (7) | 80% | 100% | 90% |
| S4A-137 (7) | 100% | 100% | 100% |
| S4A-138 (7) | 86% | 100% | 100% |
| S4A-140 (7) | 100% | 100% | 100% |
| S4A-149 (7) | 89% | 100% | 100% |
| S4A-162 (7) | 50% | 75% | 100% |
| S4A-163 (7) | 78% | 100% | 100% |
| S4A-165 (7) | 90% | 100% | 100% |
| S4A-167 (7) | 91% | 100% | 100% |
| S4A-168 (6) | 92% | 100% | 100% |
| S4A-170 (7) | 60% | 90% | 90% |
| S4A-171 (6) | 89% | 100% | 100% |
| S4A-176 (7) | 70% | 90% | 100% |
| S4A-180 (3) | 86% | 86% | 86% |
| S4A-183 (5)* | 0% | 100% | 100% |
| S4A-186 (6) | 80% | 100% | 100% |
| S4A-194 (7) | 89% | 100% | 100% |
| S4A-196 (7) | 50% | 83% | 83% |
| S4A-198 (7) | 88% | 100% | 100% |
| S4A-201 (7) | 75% | 100% | 100% |
| S4A-210 (7) | 86% | 100% | 100% |
| S4A-214 (7) | 86% | 100% | 100% |
| S4A-217 (7) | 64% | 100% | 100% |
| S4A-227 (6)* | 25% | 75% | 100% |
| S4A-233 (7) | 40% | 90% | 90% |
| S4A-234 (7) | 50% | 100% | 100% |
| S4A-236 (6) | 83% | 100% | 100% |
| S4A-237 (7) | 67% | 89% | 89% |
| S4A-246 (7) | 82% | 82% | 100% |
| S4A-253 (6) | 100% | 100% | 100% |
| S4A-260 (6) | 70% | 90% | 100% |
| S4A-274 (7) | 57% | 86% | 100% |
| S4A-276 (7) | 60% | 90% | 90% |
| S4A-277 (7) | 71% | 86% | 86% |
| S4A-278 (5)* | 43% | 57% | 100% |
| S4A-293 (3)* | 17% | 60% | 100% |
| S4A-307 (7) | 20% | 60% | 60% |
| S4A-309 (5) | 100% | 100% | 100% |
| S4A-334 (5)* | 29% | 71% | 100% |
| S4A-336 (6)* | 50% | 90% | 100% |
| S4A-353 (7) | 92% | 92% | 100% |
| S4A-358 (7) | 100% | 100% | 100% |
| S4A-382 (7) | 100% | 100% | 100% |
| S4A-390 (6)* | 29% | 57% | 100% |
| S4A-400 (7) | 78% | 89% | 100% |
| S4A-402 (7) | 91% | 91% | 91% |
| S4A-411 (7) | 88% | 88% | 100% |
| S4A-425 (7) | 100% | 100% | 100% |
| S4A-426 (7) | 100% | 100% | 100% |
| S4A-437 (7) | 100% | 100% | 100% |
| S4A-441 (7) | 70% | 78% | 100% |
| S4A-443 (7) | 90% | 100% | 100% |
| S4A-444 (7) | 86% | 100% | 100% |
| S4A-445 (7) | 100% | 100% | 100% |
| S4A-446 (7) | 91% | 100% | 100% |
| S4A-448 (7) | 100% | 100% | 100% |
| S4A-458 (7) | 60% | 80% | 100% |
| S4A-480 (5) | 80% | 100% | 100% |

TABLE 8b
Field flowering data collected in Oxnard, California
in 2022-2023 Oxnard, California 2022-2023

| Event (# edited | 5 Weeks | 7 Weeks | 11 weeks | 14 weeks |
|---|---|---|---|---|
| alleles) | % flowering | % flowering | % flowering | % flowering |
| WT Control (0) | 0% | 2% | 49% | 99% |
| S4A-018 (5) | 72% | 91% | 100% | 100% |
| S4A-031 (7) | 77% | 85% | 90% | 100% |
| S4A-048 (7) | 63% | 84% | 100% | 100% |
| S4A-049 (6) | 72% | 82% | 84% | 95% |
| S4A-051 (7) | 56% | 64% | 74% | 94% |
| S4A-098 (4)* | 0% | 3% | 41% | 97% |
| S4A-109 (7) | 74% | 86% | 91% | 97% |
| S4A-115 (6) | 68% | 93% | 95% | 100% |
| S4A-119 (5)* | 0% | 5% | 68% | 100% |
| S4A-123 (4)* | 0% | 10% | 77% | 100% |
| S4A-134 (7) | 66% | 85% | 94% | 100% |
| S4A-138 (7) | 68% | 84% | 84% | 100% |
| S4A-149 (7) | 56% | 82% | 82% | 95% |
| S4A-154 (7) | 84% | 87% | 97% | 100% |
| S4A-162 (7) | 63% | 85% | 97% | 100% |
| S4A-165 (7) | 66% | 76% | 85% | 100% |
| S4A-168 (6) | 72% | 79% | 97% | 100% |
| S4A-171 (6) | 58% | 76% | 84% | 100% |
| S4A-176 (7) | 79% | 84% | 84% | 100% |
| S4A-180 (3) | 47% | 81% | 100% | 100% |
| S4A-183 (5)* | 0% | 0% | 53% | 90% |
| S4A-186 (6) | 74% | 89% | 95% | 97% |
| S4A-198 (7) | 70% | 80% | 88% | 91% |
| S4A-209 (7) | 67% | 79% | 89% | 100% |
| S4A-210 (7) | 82% | 95% | 95% | 100% |
| S4A-214 (7) | 65% | 79% | 84% | 100% |
| S4A-227 (6)* | 0% | 5% | 50% | 100% |
| S4A-234 (7) | 60% | 79% | 92% | 98% |
| S4A-274 (7) | 68% | 81% | 81% | 100% |
| S4A-293 (3)* | 0% | 0% | 63% | 100% |
| S4A-307 (7) | 77% | 95% | 100% | 100% |
| S4A-334 (5)* | 0% | 0% | 88% | 100% |
| S4A-336 (6)* | 0% | 0% | 43% | 100% |
| S4A-353 (7) | 61% | 68% | 87% | 100% |
| S4A-358 (7) | 58% | 68% | 70% | 100% |
| S4A-378 (6)* | 0% | 8% | 55% | 98% |
| S4A-390 (6)* | 5% | 5% | 38% | 97% |
| S4A-391 (5) | 55% | 65% | 85% | 98% |
| S4A-402 (7) | 68% | 84% | 98% | 100% |
| S4A-411 (7) | 77% | 80% | 91% | 100% |
| S4A-425 (7) | 79% | 84% | 86% | 95% |
| S4A-426 (7) | 67% | 86% | 89% | 97% |
| S4A-437 (7) | 82% | 89% | 89% | 100% |
| S4A-443 (7) | 68% | 73% | 84% | 100% |
| S4A-444 (7) | 78% | 95% | 98% | 100% |
| S4A-445 (7) | 69% | 84% | 92% | 97% |
| S4A-448 (7) | 48% | 70% | 88% | 100% |
| S4A-458 (7) | 79% | 92% | 94% | 100% |
| S4A-480 (5) | 70% | 75% | 90% | 100% |

TABLE 8c
Field flowering data collected in
Oxnard, California in summer 2023

Oxnard, California 2023

| | 6 Weeks | 7 Weeks |
|---|---|---|
| Event (# edited alleles) | % flowering | % flowering |
| WT Control (0) | 8% | 9% |
| S4A-018 (5) | 65% | 100% |
| S4A-019 (7) | 74% | 95% |
| S4A-029 (7) | 36% | 88% |
| S4A-031 (7) | 75% | 100% |
| S4A-048 (7) | 75% | 100% |
| S4A-049 (6) | 74% | 95% |
| S4A-051 (7) | 63% | 100% |
| S4A-098 (4)* | 10% | 43% |
| S4A-115 (6) | 58% | 98% |
| S4A-119 (5)* | 15% | 43% |
| S4A-123 (4)* | 10% | 13% |
| S4A-134 (7) | 78% | 100% |
| S4A-154 (7) | 71% | 100% |
| S4A-162 (7) | 70% | 100% |
| S4A-165 (7) | 85% | 100% |
| S4A-180 (3) | 48% | 100% |
| S4A-098 (5)* | 0% | 15% |
| S4A-186 (6) | 65% | 95% |
| S4A-194 (7) | 70% | 100% |
| S4A-209 (7) | 69% | 97% |
| S4A-210 (7) | 53% | 100% |
| S4A-214 (7) | 65% | 100% |
| S4A-227 (6)* | 13% | 23% |
| S4A-293 (3)* | 8% | 10% |
| S4A-307 (7) | 58% | 98% |
| S4A-334 (5)* | 8% | 15% |
| S4A-336 (6)* | 10% | 23% |
| S4A-353 (7) | 80% | 100% |
| S4A-358 (7) | 70% | 100% |
| S4A-378 (6)* | 8% | 28% |
| S4A-390 (6)* | 5% | 25% |
| S4A-391 (5) | 70% | 100% |
| S4A-402 (7) | 63% | 95% |
| S4A-426 (7) | 66% | 100% |
| S4A-437 (7) | 85% | 100% |
| S4A-443 (7) | 63% | 98% |
| S4A-444 (7) | 87% | 100% |
| S4A-445 (7) | 73% | 100% |
| S4A-448 (7) | 68% | 95% |
| S4A-480 (5) | 63% | 100% |

### Example 4: Field Trials—Yield

Plants were grown and transplanted to fields as described above in Example 3. Fruit was hand harvested twice a week and documented as marketable or cull. Total fruit was included as marketable and cull fruit and is shown in the Tables below as grams per plant (g/plant) or percent increase compared to wildtype yield. For the 2021-22 trials plants were not conditioned or vernalized before planting.

TABLE 9a
Field yield data for Salinas, California 2022

| | April | | May | | June | | July | | August | | September | |
|---|---|---|---|---|---|---|---|---|---|---|---|---|
| | g/ | % | g/ | % | g/ | % | g/ | % | g/ | % | g/ | % |
| | plant | change | plant | change | plant | change | plant | change | plant | change | plant | change |
| WT Control | 70 | 0% | 398 | 0% | 556 | 0% | 304 | 0% | 137 | 0% | 189 | 0% |
| S4A-008 | 66 | −6% | 395 | −1% | 564 | 1% | 441 | 45% | 395 | 189% | 430 | 127% |
| S4A-018 | 61 | −13% | 387 | −3% | 665 | 20% | 542 | 78% | 481 | 251% | 298 | 57% |
| S4A-019 | 85 | 21% | 348 | −13% | 532 | −4% | 747 | 146% | 532 | 289% | 377 | 99% |
| S4A-020 | 24 | −65% | 186 | −53% | 787 | 42% | 539 | 77% | 473 | 245% | 262 | 38% |
| S4A-024 | 59 | −16% | 484 | 21% | 679 | 22% | 420 | 38% | 393 | 187% | 280 | 48% |
| S4A-029 | 63 | −10% | 264 | −34% | 609 | 10% | 498 | 64% | 448 | 227% | 587 | 210% |
| S4A-031 | 73 | 5% | 470 | 18% | 787 | 42% | 639 | 110% | 436 | 218% | 400 | 111% |
| S4A-046 | 57 | −19% | 474 | 19% | 661 | 19% | 610 | 100% | 365 | 167% | 252 | 33% |
| S4A-047 | 46 | −35% | 279 | −30% | 767 | 38% | 478 | 57% | 429 | 213% | 381 | 101% |
| S4A-048 | 61 | −13% | 346 | −13% | 704 | 27% | 624 | 105% | 469 | 242% | 458 | 142% |
| S4A-049 | 33 | −54% | 249 | −38% | 821 | 48% | 637 | 109% | 460 | 236% | 317 | 67% |
| S4A-051 | 70 | −1% | 616 | 55% | 802 | 44% | 462 | 52% | 312 | 128% | 347 | 83% |
| S4A-073 | 60 | −15% | 343 | −14% | 703 | 26% | 741 | 143% | 361 | 163% | 255 | 35% |
| S4A-076 | 24 | −65% | 97 | −76% | 780 | 40% | 454 | 49% | 175 | 27% | 153 | −19% |
| S4A-098 | 71 | 2% | 233 | −41% | 531 | −5% | 463 | 52% | 271 | 98% | 479 | 153% |
| S4A-108 | 10 | −85% | 148 | −63% | 827 | 49% | 563 | 85% | 209 | 52% | 319 | 68% |
| S4A-109 | 61 | −14% | 325 | −18% | 612 | 10% | 487 | 60% | 447 | 226% | 270 | 43% |
| S4A-115 | 50 | −29% | 362 | −9% | 704 | 27% | 550 | 81% | 434 | 217% | 499 | 163% |
| S4A-119 | 35 | −50% | 128 | −68% | 718 | 29% | 585 | 92% | 252 | 84% | 265 | 40% |
| S4A-123 | 61 | −13% | 221 | −45% | 292 | −47% | 217 | −29% | 93 | −32% | 102 | −46% |
| S4A-126 | 51 | −27% | 191 | −52% | 614 | 10% | 611 | 101% | 431 | 215% | 428 | 126% |
| S4A-130 | 22 | −69% | 137 | −66% | 418 | −25% | 201 | −34% | 54 | −61% | 90 | −53% |
| S4A-134 | 118 | 69% | 430 | 8% | 904 | 63% | 904 | 197% | 562 | 310% | 373 | 97% |
| S4A-137 | 57 | −18% | 200 | −50% | 370 | −33% | 414 | 36% | 150 | 9% | 97 | −49% |
| S4A-138 | 54 | −24% | 318 | −20% | 626 | 13% | 602 | 98% | 414 | 203% | 373 | 97% |
| S4A-149 | 128 | 83% | 535 | 34% | 614 | 10% | 531 | 75% | 317 | 131% | 405 | 114% |
| S4A-163 | 37 | −47% | 334 | −16% | 767 | 38% | 520 | 71% | 386 | 182% | 411 | 117% |
| S4A-165 | 49 | −30% | 180 | −55% | 791 | 42% | 740 | 143% | 680 | 396% | 597 | 215% |
| S4A-167 | 27 | −62% | 111 | −72% | 650 | 17% | 607 | 99% | 525 | 284% | 541 | 185% |
| S4A-168 | 48 | −31% | 182 | −54% | 915 | 64% | 777 | 155% | 795 | 480% | 856 | 352% |
| S4A-170 | 47 | −32% | 296 | −26% | 496 | −11% | 475 | 56% | 261 | 91% | 225 | 19% |
| S4A-171 | 150 | 114% | 669 | 68% | 668 | 20% | 513 | 69% | 288 | 110% | 356 | 88% |
| S4A-176 | 102 | 46% | 709 | 78% | 778 | 40% | 651 | 114% | 477 | 248% | 397 | 110% |
| S4A-180 | 20 | −71% | 114 | −71% | 634 | 14% | 693 | 128% | 282 | 106% | 551 | 191% |
| S4A-186 | 39 | −45% | 199 | −50% | 685 | 23% | 859 | 182% | 407 | 197% | 459 | 142% |
| S4A-194 | 46 | −34% | 214 | −46% | 596 | 7% | 637 | 109% | 306 | 123% | 251 | 33% |
| S4A-196 | 31 | −56% | 196 | −51% | 717 | 29% | 527 | 73% | 274 | 100% | 434 | 129% |
| S4A-201 | 18 | −74% | 52 | −87% | 0 | −100% | 0 | −100% | 0 | −100% | 0 | −100% |
| S4A-210 | 39 | −44% | 370 | −7% | 623 | 12% | 577 | 90% | 359 | 162% | 531 | 180% |
| S4A-214 | 109 | 55% | 668 | 68% | 740 | 33% | 484 | 59% | 328 | 139% | 432 | 128% |
| S4A-227 | 60 | −15% | 305 | −23% | 643 | 16% | 393 | 29% | 257 | 87% | 330 | 74% |
| S4A-233 | 87 | 24% | 327 | −18% | 588 | 6% | 502 | 65% | 277 | 102% | 299 | 58% |
| S4A-234 | 124 | 77% | 302 | −24% | 567 | 2% | 559 | 84% | 298 | 118% | 340 | 79% |
| S4A-236 | 55 | −21% | 200 | −50% | 770 | 38% | 639 | 110% | 586 | 328% | 425 | 124% |
| S4A-253 | 42 | −40% | 214 | −46% | 639 | 15% | 508 | 67% | 391 | 186% | 434 | 129% |
| S4A-274 | 68 | −3% | 485 | 22% | 756 | 36% | 624 | 105% | 391 | 186% | 548 | 189% |
| S4A-293 | 93 | 33% | 552 | 39% | 696 | 25% | 248 | −18% | 180 | 32% | 310 | 64% |
| S4A-307 | 106 | 51% | 680 | 71% | 716 | 29% | 493 | 62% | 381 | 178% | 541 | 186% |
| S4A-309 | 23 | −68% | 292 | −27% | 527 | −5% | 436 | 43% | 258 | 89% | 292 | 54% |
| S4A-336 | 57 | −19% | 382 | −4% | 655 | 18% | 367 | 21% | 216 | 58% | 284 | 50% |
| S4A-353 | 58 | −18% | 285 | −28% | 636 | 14% | 502 | 65% | 228 | 66% | 351 | 85% |
| S4A-358 | 60 | −15% | 395 | −1% | 820 | 47% | 557 | 83% | 473 | 245% | 425 | 125% |
| S4A-400 | 32 | −54% | 253 | −37% | 487 | −12% | 277 | −9% | 196 | 43% | 154 | −19% |
| S4A-402 | 63 | −10% | 384 | −3% | 681 | 22% | 530 | 74% | 384 | 180% | 432 | 128% |
| S4A-411 | 60 | −15% | 330 | −17% | 728 | 31% | 582 | 91% | 318 | 132% | 438 | 131% |
| S4A-425 | 56 | −21% | 196 | −51% | 890 | 60% | 898 | 195% | 724 | 429% | 719 | 280% |
| S4A-426 | 171 | 144% | 1023 | 157% | 508 | −9% | 598 | 96% | 282 | 106% | 365 | 92% |
| S4A-437 | 61 | −13% | 302 | −24% | 590 | 6% | 393 | 29% | 251 | 83% | 360 | 90% |
| S4A-444 | 75 | 7% | 401 | 1% | 661 | 19% | 526 | 73% | 359 | 162% | 474 | 150% |
| S4A-445 | 121 | 72% | 690 | 73% | 614 | 10% | 493 | 62% | 358 | 161% | 425 | 124% |
| S4A-448 | 187 | 166% | 1610 | 304% | 722 | 30% | 479 | 57% | 390 | 184% | 521 | 175% |
| S4A-479 | 6 | −91% | 37 | −91% | 410 | −26% | 325 | 7% | 147 | 8% | 112 | −41% |

TABLE 9b
Field yield data for Salinas, California 2023

| | April | | May | | June | | July | | August | | September | |
|---|---|---|---|---|---|---|---|---|---|---|---|---|
| | g/ | % | g/ | % | g/ | % | g/ | % | g/ | % | g/ | % |
| | plant | change | plant | change | plant | change | plant | change | plant | change | plant | change |
| WT Control | 55 | 0% | 313 | 0% | 499 | 0% | 184 | 0% | 139 | 0% | 233 | 0% |
| S4A-018 | 51 | −7% | 373 | 19% | 589 | 18% | 414 | 125% | 393 | 181% | 412 | 77% |
| S4A-019 | 29 | −47% | 278 | −11% | 501 | 0% | 385 | 109% | 360 | 158% | 343 | 47% |
| S4A-029 | 47 | −14% | 383 | 22% | 584 | 17% | 480 | 161% | 439 | 215% | 433 | 86% |
| S4A-031 | 38 | −31% | 297 | −5% | 575 | 15% | 370 | 101% | 390 | 180% | 380 | 63% |
| S4A-048 | 46 | −16% | 313 | 0% | 571 | 14% | 348 | 89% | 326 | 133% | 340 | 46% |
| S4A-049 | 29 | −48% | 293 | −7% | 590 | 18% | 401 | 118% | 424 | 204% | 443 | 90% |
| S4A-051 | 63 | 15% | 271 | −14% | 568 | 14% | 340 | 85% | 421 | 202% | 414 | 78% |
| S4A-098 | 71 | 29% | 358 | 14% | 502 | 1% | 224 | 22% | 237 | 70% | 308 | 32% |
| S4A-109 | 28 | −49% | 243 | −22% | 376 | −25% | 280 | 52% | 337 | 142% | 329 | 41% |
| S4A-115 | 39 | −28% | 300 | −4% | 548 | 10% | 412 | 124% | 431 | 209% | 428 | 84% |
| S4A-119 | 58 | 6% | 390 | 24% | 582 | 17% | 242 | 32% | 169 | 21% | 282 | 21% |
| S4A-123 | 66 | 21% | 350 | 12% | 565 | 13% | 256 | 39% | 198 | 42% | 278 | 19% |
| S4A-134 | 48 | −12% | 385 | 23% | 602 | 21% | 326 | 77% | 417 | 199% | 424 | 82% |
| S4A-138 | 35 | −37% | 279 | −11% | 532 | 7% | 396 | 116% | 361 | 159% | 348 | 49% |
| S4A-149 | 42 | −24% | 333 | 6% | 509 | 2% | 368 | 100% | 382 | 174% | 439 | 89% |
| S4A-154 | 46 | −16% | 273 | −13% | 560 | 12% | 423 | 130% | 368 | 164% | 394 | 69% |
| S4A-162 | 48 | −12% | 304 | −3% | 545 | 9% | 393 | 114% | 406 | 191% | 382 | 64% |
| S4A-165 | 43 | −21% | 346 | 10% | 701 | 41% | 373 | 103% | 422 | 202% | 439 | 88% |
| S4A-168 | 55 | 1% | 319 | 2% | 587 | 18% | 358 | 95% | 392 | 181% | 512 | 120% |
| S4A-171 | 26 | −53% | 315 | 1% | 637 | 28% | 370 | 101% | 401 | 187% | 446 | 92% |
| S4A-176 | 51 | −7% | 348 | 11% | 614 | 23% | 421 | 129% | 524 | 276% | 549 | 136% |
| S4A-180 | 83 | 51% | 330 | 5% | 516 | 4% | 293 | 59% | 366 | 163% | 334 | 43% |
| S4A-183 | 24 | −56% | 180 | −43% | 439 | −12% | 246 | 34% | 237 | 70% | 305 | 31% |
| S4A-186 | 54 | −1% | 371 | 18% | 695 | 39% | 308 | 67% | 389 | 179% | 392 | 68% |
| S4A-194 | 44 | −19% | 284 | −9% | 517 | 4% | 310 | 69% | 351 | 152% | 340 | 46% |
| S4A-198 | 27 | −51% | 289 | −8% | 567 | 14% | 338 | 84% | 393 | 182% | 370 | 59% |
| S4A-209 | 29 | −47% | 355 | 13% | 575 | 15% | 352 | 91% | 453 | 225% | 416 | 78% |
| S4A-210 | 40 | −28% | 314 | 0% | 679 | 36% | 453 | 147% | 383 | 174% | 459 | 97% |
| S4A-214 | 36 | −35% | 299 | −4% | 513 | 3% | 360 | 96% | 388 | 178% | 433 | 86% |
| S4A-227 | 62 | 13% | 331 | 6% | 534 | 7% | 253 | 37% | 182 | 31% | 220 | −6% |
| S4A-234 | 52 | −5% | 263 | −16% | 564 | 13% | 345 | 88% | 372 | 167% | 403 | 73% |
| S4A-274 | 36 | −34% | 332 | 6% | 671 | 35% | 317 | 72% | 347 | 149% | 309 | 33% |
| S4A-293 | 63 | 16% | 366 | 17% | 507 | 2% | 174 | −5% | 147 | 5% | 224 | −4% |
| S4A-307 | 49 | −11% | 261 | −17% | 474 | −5% | 378 | 106% | 414 | 197% | 409 | 76% |
| S4A-334 | 73 | 33% | 402 | 28% | 620 | 24% | 374 | 103% | 328 | 135% | 406 | 74% |
| S4A-336 | 57 | 3% | 259 | −17% | 509 | 2% | 279 | 52% | 268 | 92% | 261 | 12% |
| S4A-353 | 40 | −27% | 317 | 1% | 645 | 29% | 365 | 99% | 344 | 147% | 376 | 61% |
| S4A-358 | 39 | −29% | 261 | −17% | 656 | 31% | 395 | 115% | 413 | 196% | 430 | 85% |
| S4A-378 | 73 | 33% | 298 | −5% | 570 | 14% | 314 | 71% | 307 | 120% | 298 | 28% |
| S4A-390 | 40 | −26% | 303 | −3% | 539 | 8% | 258 | 40% | 231 | 66% | 226 | −3% |
| S4A-391 | 22 | −60% | 276 | −12% | 671 | 34% | 349 | 90% | 351 | 152% | 337 | 45% |
| S4A-402 | 28 | −50% | 305 | −3% | 573 | 15% | 363 | 98% | 398 | 185% | 398 | 71% |
| S4A-411 | 39 | −29% | 325 | 4% | 501 | 0% | 352 | 92% | 445 | 219% | 386 | 66% |
| S4A-425 | 47 | −15% | 320 | 2% | 564 | 13% | 430 | 134% | 446 | 219% | 396 | 70% |
| S4A-426 | 33 | −39% | 251 | −20% | 581 | 16% | 331 | 80% | 384 | 175% | 434 | 86% |
| S4A-437 | 29 | −47% | 277 | −12% | 582 | 17% | 429 | 134% | 374 | 168% | 463 | 99% |
| S4A-443 | 39 | −28% | 292 | −7% | 614 | 23% | 376 | 104% | 407 | 192% | 483 | 107% |
| S4A-444 | 46 | −15% | 291 | −7% | 543 | 9% | 358 | 95% | 391 | 180% | 391 | 68% |
| S4A-445 | 51 | −6% | 380 | 21% | 627 | 26% | 393 | 114% | 411 | 195% | 420 | 80% |
| S4A-448 | 60 | 9% | 231 | −26% | 709 | 42% | 491 | 167% | 367 | 163% | 324 | 39% |
| S4A-458 | 44 | −19% | 299 | −4% | 582 | 17% | 429 | 133% | 437 | 214% | 431 | 85% |
| S4A-480 | 60 | 9% | 354 | 13% | 585 | 17% | 309 | 68% | 398 | 186% | 379 | 63% |

TABLE 10a
Field yield data for Oxnard, California 2021-2022

| | December | | January | | February | | March | | April | | May | | June | |
|---|---|---|---|---|---|---|---|---|---|---|---|---|---|---|
| | g/ | % | g/ | % | g/ | % | g/ | % | g/ | % | g/ | % | g/ | % |
| | plant | change | plant | change | plant | change | plant | change | plant | change | plant | change | plant | change |
| WT | 2 | — | 31 | 0% | 123 | 0% | 202 | 0% | 268 | 0% | 246 | 0% | 108 | 0% |
| Control | | | | | | | | | | | | | | |
| S4A-008 | 0 | — | 18 | −44% | 29 | −77% | 93 | −54% | 172 | −36% | 192 | −22% | 113 | 4% |
| S4A-018 | 3 | — | 7 | −76% | 61 | −50% | 122 | −39% | 141 | −47% | 225 | −8% | 132 | 22% |
| S4A-019 | 9 | — | 66 | 112% | 86 | −30% | 121 | −40% | 147 | −45% | 179 | −27% | 64 | −41% |
| S4A-020 | 8 | — | 40 | 29% | 73 | −41% | 137 | −32% | 186 | −31% | 158 | −36% | 61 | −43% |
| S4A-024 | 7 | — | 34 | 10% | 71 | −42% | 114 | −43% | 144 | −46% | 214 | −13% | 145 | 34% |
| S4A-027 | 0 | — | 17 | −44% | 55 | −55% | 87 | −57% | 172 | −36% | 194 | −21% | 136 | 26% |
| S4A-029 | 11 | — | 69 | 120% | 99 | −20% | 133 | −34% | 207 | −23% | 230 | −6% | 83 | −24% |
| S4A-031 | 7 | — | 55 | 76% | 107 | −13% | 136 | −33% | 160 | −40% | 186 | −24% | 141 | 31% |
| S4A-046 | 0 | — | 24 | −23% | 94 | −23% | 133 | −34% | 191 | −29% | 202 | −18% | 124 | 15% |
| S4A-047 | 5 | — | 38 | 23% | 68 | −45% | 119 | −41% | 206 | −23% | 169 | −31% | 39 | −64% |
| S4A-048 | 0 | — | 47 | 51% | 106 | −14% | 142 | −29% | 191 | −29% | 272 | 11% | 103 | −5% |
| S4A-049 | 6 | — | 49 | 55% | 95 | −23% | 128 | −37% | 189 | −29% | 201 | −18% | 104 | −4% |
| S4A-051 | 5 | — | 38 | 23% | 81 | −34% | 116 | −43% | 159 | −41% | 230 | −6% | 121 | 11% |
| S4A-065 | 2 | — | 23 | −27% | 45 | −63% | 90 | −55% | 127 | −52% | 278 | 13% | 97 | −11% |
| S4A-072 | 0 | — | 15 | −51% | 74 | −40% | 116 | −42% | 136 | −49% | 162 | −34% | 125 | 15% |
| S4A-073 | 4 | — | 29 | −7% | 70 | −43% | 112 | −44% | 168 | −37% | 228 | −7% | 140 | 30% |
| S4A-076 | 4 | — | 18 | −43% | 52 | −57% | 100 | −50% | 125 | −53% | 162 | −34% | 67 | −38% |
| S4A-098 | 5 | — | 48 | 54% | 149 | 21% | 169 | −16% | 244 | −9% | 207 | −16% | 75 | −31% |
| S4A-108 | 0 | — | 16 | −48% | 72 | −42% | 148 | −27% | 188 | −30% | 218 | −11% | 102 | −5% |
| S4A-109 | 9 | — | 39 | 25% | 107 | −13% | 139 | −31% | 205 | −23% | 178 | −28% | 117 | 8% |
| S4A-115 | 0 | — | 25 | −21% | 253 | 105% | 302 | 50% | 411 | 53% | 313 | 27% | 90 | −17% |
| S4A-123 | 0 | — | 53 | 71% | 196 | 60% | 237 | 17% | 330 | 23% | 244 | −1% | 100 | −7% |
| S4A-126 | 5 | — | 41 | 33% | 74 | −40% | 151 | −25% | 218 | −19% | 229 | −7% | 101 | −7% |
| S4A-130 | 3 | — | 37 | 18% | 98 | −20% | 206 | 2% | 215 | −20% | 189 | −23% | 56 | −48% |
| S4A-134 | 0 | — | 24 | −22% | 55 | −55% | 102 | −49% | 116 | −57% | 97 | −61% | 41 | −62% |
| S4A-137 | 3 | — | 42 | 36% | 69 | −44% | 85 | −58% | 107 | −60% | 119 | −52% | 66 | −39% |
| S4A-138 | 7 | — | 42 | 35% | 92 | −26% | 116 | −43% | 123 | −54% | 168 | −32% | 101 | −7% |
| S4A-140 | 0 | — | 32 | 3% | 63 | −49% | 72 | −64% | 119 | −56% | 139 | −44% | 61 | −43% |
| S4A-149 | 17 | — | 44 | 42% | 84 | −31% | 109 | −46% | 160 | −40% | 208 | −15% | 151 | 39% |
| S4A-162 | 0 | — | 29 | −6% | 94 | −24% | 110 | −45% | 236 | −12% | 287 | 17% | 189 | 75% |
| S4A-163 | 9 | — | 38 | 20% | 99 | −19% | 138 | −32% | 135 | −50% | 229 | −7% | 104 | −4% |
| S4A-165 | 14 | — | 38 | 22% | 69 | −44% | 107 | −47% | 159 | −40% | 205 | −16% | 99 | −9% |
| S4A-167 | 0 | — | 56 | 79% | 78 | −37% | 101 | −50% | 158 | −41% | 255 | 4% | 141 | 31% |
| S4A-168 | 17 | — | 78 | 149% | 128 | 4% | 172 | −15% | 195 | −27% | 207 | −16% | 93 | −14% |
| S4A-170 | 4 | — | 32 | 3% | 87 | −29% | 131 | −35% | 162 | −40% | 197 | −20% | 59 | −45% |
| S4A-171 | 0 | — | 24 | −22% | 70 | −43% | 122 | −40% | 236 | −12% | 251 | 2% | 128 | 18% |
| S4A-176 | 4 | — | 26 | −16% | 72 | −42% | 145 | −28% | 144 | −46% | 207 | −16% | 93 | −14% |
| S4A-180 | 5 | — | 32 | 2% | 52 | −58% | 93 | −54% | 163 | −39% | 208 | −15% | 98 | −10% |
| S4A-183 | 0 | — | 9 | −71% | 181 | 47% | 358 | 77% | 448 | 67% | 412 | 68% | 128 | 18% |
| S4A-186 | 13 | — | 53 | 71% | 85 | −31% | 179 | −11% | 260 | −3% | 227 | −8% | 94 | −13% |
| S4A-194 | 7 | — | 53 | 71% | 98 | −21% | 187 | −7% | 217 | −19% | 270 | 10% | 102 | −5% |
| S4A-196 | 10 | — | 23 | −28% | 68 | −45% | 113 | −44% | 170 | −37% | 163 | −34% | 62 | −42% |
| S4A-198 | 8 | — | 41 | 31% | 108 | −12% | 207 | 3% | 176 | −34% | 222 | −10% | 93 | −14% |
| S4A-201 | 0 | — | 8 | −73% | 42 | −66% | 156 | −23% | 277 | 3% | 217 | −12% | 182 | 68% |
| S4A-210 | 4 | — | 52 | 65% | 126 | 2% | 184 | −9% | 241 | −10% | 301 | 23% | 147 | 35% |
| S4A-214 | 0 | — | 15 | −51% | 46 | −62% | 129 | −36% | 148 | −45% | 217 | −12% | 74 | −31% |
| S4A-217 | 5 | — | 23 | −25% | 57 | −54% | 144 | −29% | 138 | −48% | 205 | −17% | 84 | −22% |
| S4A-227 | 8 | — | 46 | 46% | 104 | −16% | 208 | 3% | 225 | −16% | 254 | 3% | 95 | −12% |
| S4A-233 | 0 | — | 25 | −19% | 87 | −29% | 139 | −31% | 178 | −34% | 189 | −23% | 97 | −11% |
| S4A-234 | 0 | — | 0 | −100% | 68 | −45% | 204 | 1% | 318 | 19% | 317 | 29% | 159 | 46% |
| S4A-236 | 0 | — | 40 | 29% | 68 | −45% | 157 | −22% | 184 | −31% | 176 | −28% | 55 | −49% |
| S4A-237 | 3 | — | 13 | −59% | 41 | −66% | 139 | −31% | 183 | −32% | 276 | 12% | 102 | −6% |
| S4A-246 | 0 | — | 0 | −100% | 9 | −92% | 40 | −80% | 90 | −67% | 128 | −48% | 32 | −71% |
| S4A-253 | 10 | — | 72 | 131% | 124 | 1% | 164 | −19% | 185 | −31% | 222 | −10% | 109 | 0% |
| S4A-260 | 0 | — | 0 | −100% | 18 | −86% | 76 | −62% | 125 | −53% | 164 | −33% | 78 | −28% |
| S4A-274 | 3 | — | 25 | −20% | 74 | −40% | 128 | −37% | 171 | −36% | 282 | 15% | 215 | 98% |
| S4A-276 | 5 | — | 43 | 37% | 90 | −27% | 133 | −34% | 144 | −46% | 157 | −36% | 66 | −39% |
| S4A-277 | 0 | — | 14 | −54% | 11 | −91% | 57 | −72% | 114 | −58% | 160 | −35% | 75 | −31% |
| S4A-278 | 4 | — | 14 | −55% | 81 | −34% | 188 | −7% | 213 | −20% | 251 | 2% | 76 | −30% |
| S4A-293 | 0 | — | 8 | −74% | 62 | −50% | 198 | −2% | 349 | 30% | 324 | 32% | 102 | −6% |
| S4A-307 | 0 | — | 0 | −100% | 34 | −72% | 126 | −38% | 213 | −20% | 303 | 23% | 111 | 2% |
| S4A-309 | 14 | — | 30 | −3% | 60 | −51% | 127 | −37% | 176 | −34% | 199 | −19% | 80 | −26% |
| S4A-334 | 0 | — | 37 | 20% | 129 | 5% | 213 | 5% | 285 | 6% | 255 | 4% | 115 | 6% |
| S4A-336 | 6 | — | 47 | 50% | 143 | 17% | 237 | 18% | 348 | 30% | 282 | 15% | 131 | 21% |
| S4A-353 | 0 | — | 63 | 103% | 107 | −13% | 118 | −41% | 129 | −52% | 175 | −29% | 140 | 30% |
| S4A-358 | 4 | — | 27 | −13% | 74 | −40% | 148 | −27% | 181 | −32% | 215 | −13% | 142 | 31% |
| S4A-382 | 16 | — | 33 | 4% | 72 | −42% | 140 | −31% | 122 | −55% | 203 | −18% | 99 | −9% |
| S4A-390 | 4 | — | 23 | −28% | 130 | 6% | 265 | 31% | 262 | −2% | 195 | −21% | 67 | −38% |
| S4A-400 | 3 | — | 60 | 92% | 96 | −22% | 136 | −33% | 162 | −39% | 214 | −13% | 91 | −16% |
| S4A-402 | 12 | — | 32 | 3% | 104 | −15% | 183 | −9% | 211 | −21% | 215 | −12% | 94 | −13% |
| S4A-411 | 7 | — | 47 | 52% | 64 | −48% | 117 | −42% | 166 | −38% | 237 | −4% | 78 | −28% |
| S4A-425 | 14 | — | 47 | 51% | 114 | −7% | 167 | −17% | 180 | −33% | 217 | −12% | 99 | −9% |
| S4A-426 | 9 | — | 57 | 81% | 77 | −38% | 121 | −40% | 201 | −25% | 255 | 4% | 82 | −24% |
| S4A-437 | 19 | — | 51 | 62% | 79 | −35% | 126 | −37% | 178 | −34% | 219 | −11% | 108 | 0% |
| S4A-441 | 6 | — | 36 | 14% | 73 | −41% | 147 | −27% | 203 | −24% | 263 | 7% | 121 | 12% |
| S4A-443 | 14 | — | 63 | 101% | 119 | −4% | 148 | −27% | 188 | −30% | 208 | −15% | 105 | −3% |
| S4A-444 | 18 | — | 50 | 61% | 142 | 15% | 237 | 18% | 250 | −7% | 288 | 17% | 120 | 10% |
| S4A-445 | 21 | — | 70 | 125% | 110 | −10% | 172 | −15% | 197 | −27% | 212 | −14% | 147 | 36% |
| S4A-446 | 13 | — | 47 | 52% | 56 | −54% | 124 | −39% | 120 | −55% | 193 | −21% | 80 | −26% |
| S4A-448 | 10 | — | 83 | 166% | 91 | −26% | 150 | −26% | 272 | 1% | 492 | 100% | 266 | 146% |
| S4A-458 | 7 | — | 57 | 83% | 87 | −29% | 125 | −38% | 213 | −20% | 260 | 6% | 131 | 21% |
| S4A-480 | 9 | — | 47 | 51% | 115 | −7% | 241 | 20% | 286 | 7% | 296 | 21% | 111 | 2% |

TABLE 10b
Field yield data for Oxnard, California 2022-2023

| | December | | January | | February | | March | | April | | May | | June | |
|---|---|---|---|---|---|---|---|---|---|---|---|---|---|---|
| | g/ | % | g/ | % | g/ | % | g/ | % | g/ | % | g/ | % | g/ | % |
| | plant | change | plant | change | plant | change | plant | change | plant | change | plant | change | plant | change |
| WT | 0 | — | 6 | 0% | 67 | 0% | 148 | 0% | 357 | 0% | 491 | 0% | 176 | 0% |
| Control | | | | | | | | | | | | | | |
| S4A-018 | 3 | — | 28 | 409% | 39 | −42% | 118 | −20% | 266 | −25% | 481 | −2% | 210 | 19% |
| S4A-031 | 2 | — | 24 | 329% | 41 | −39% | 135 | −9% | 294 | −18% | 470 | −4% | 210 | 19% |
| S4A-048 | 6 | — | 29 | 427% | 43 | −35% | 121 | −18% | 279 | −22% | 442 | −10% | 192 | 9% |
| S4A-049 | 3 | — | 16 | 178% | 24 | −65% | 102 | −31% | 264 | −26% | 393 | −20% | 197 | 12% |
| S4A-051 | 1 | — | 14 | 156% | 24 | −64% | 105 | −29% | 243 | −32% | 463 | −6% | 214 | 22% |
| S4A-098 | 0 | — | 5 | −2% | 42 | −37% | 140 | −5% | 310 | −13% | 438 | −11% | 169 | −4% |
| S4A-109 | 8 | — | 19 | 236% | 30 | −54% | 79 | −47% | 194 | −46% | 342 | −30% | 179 | 2% |
| S4A-115 | 4 | — | 27 | 384% | 36 | −45% | 101 | −31% | 226 | −37% | 360 | −27% | 188 | 7% |
| S4A-119 | 0 | — | 9 | 60% | 62 | −7% | 144 | −3% | 343 | −4% | 447 | −9% | 188 | 7% |
| S4A-123 | 0 | — | 18 | 217% | 81 | 21% | 168 | 14% | 362 | 2% | 464 | −6% | 153 | −13% |
| S4A-134 | 4 | — | 16 | 192% | 36 | −46% | 116 | −22% | 225 | −37% | 408 | −17% | 173 | −2% |
| S4A-138 | 10 | — | 21 | 275% | 38 | −43% | 84 | −43% | 220 | −38% | 346 | −29% | 171 | −3% |
| S4A-149 | 4 | — | 12 | 123% | 26 | −62% | 92 | −38% | 208 | −42% | 355 | −28% | 173 | −1% |
| S4A-154 | 7 | — | 30 | 435% | 30 | −55% | 84 | −43% | 165 | −54% | 341 | −31% | 231 | 31% |
| S4A-162 | 2 | — | 24 | 327% | 42 | −36% | 106 | −28% | 244 | −32% | 397 | −19% | 213 | 21% |
| S4A-165 | 4 | — | 23 | 304% | 38 | −43% | 101 | −32% | 250 | −30% | 483 | −2% | 233 | 32% |
| S4A-168 | 3 | — | 20 | 262% | 40 | −40% | 117 | −21% | 241 | −33% | 456 | −7% | 209 | 19% |
| S4A-171 | 3 | — | 17 | 208% | 27 | −59% | 89 | −40% | 216 | −39% | 431 | −12% | 213 | 21% |
| S4A-176 | 6 | — | 22 | 288% | 33 | −50% | 115 | −22% | 250 | −30% | 464 | −6% | 208 | 18% |
| S4A-180 | 1 | — | 44 | 681% | 45 | −32% | 109 | −26% | 185 | −48% | 348 | −29% | 163 | −7% |
| S4A-183 | 0 | — | 3 | −42% | 66 | 0% | 145 | −2% | 306 | −14% | 539 | 10% | 195 | 11% |
| S4A-186 | 4 | — | 28 | 405% | 38 | −44% | 113 | −23% | 217 | −39% | 388 | −21% | 185 | 5% |
| S4A-198 | 3 | — | 20 | 252% | 30 | −55% | 112 | −24% | 259 | −27% | 418 | −15% | 195 | 11% |
| S4A-209 | 3 | — | 12 | 108% | 29 | −56% | 84 | −43% | 203 | −43% | 358 | −27% | 197 | 12% |
| S4A-210 | 7 | — | 25 | 344% | 33 | −51% | 90 | −39% | 216 | −39% | 401 | −18% | 206 | 17% |
| S4A-214 | 2 | — | 15 | 166% | 23 | −65% | 96 | −35% | 194 | −46% | 331 | −33% | 150 | −15% |
| S4A-227 | 0 | — | 8 | 41% | 61 | −8% | 136 | −8% | 331 | −7% | 518 | 6% | 186 | 6% |
| S4A-234 | 4 | — | 26 | 373% | 42 | −36% | 105 | −29% | 316 | −11% | 502 | 2% | 186 | 6% |
| S4A-274 | 2 | — | 12 | 114% | 27 | −60% | 91 | −39% | 184 | −48% | 331 | −33% | 144 | −18% |
| S4A-293 | 0 | — | 1 | −77% | 68 | 3% | 173 | 17% | 403 | 13% | 576 | 17% | 176 | 0% |
| S4A-307 | 5 | — | 35 | 529% | 56 | −15% | 146 | −1% | 252 | −29% | 421 | −14% | 232 | 32% |
| S4A-334 | 1 | — | 23 | 305% | 109 | 63% | 203 | 38% | 363 | 2% | 476 | −3% | 159 | −10% |
| S4A-336 | 0 | — | 5 | −10% | 46 | −31% | 145 | −2% | 292 | −18% | 433 | −12% | 140 | −21% |
| S4A-353 | 5 | — | 20 | 253% | 41 | −38% | 107 | −27% | 220 | −38% | 383 | −22% | 186 | 5% |
| S4A-358 | 2 | — | 22 | 296% | 29 | −56% | 112 | −24% | 266 | −26% | 406 | −17% | 155 | −12% |
| S4A-378 | 0 | — | 8 | 48% | 67 | 1% | 143 | −3% | 274 | −23% | 449 | −9% | 190 | 8% |
| S4A-390 | 0 | — | 1 | −84% | 52 | −22% | 130 | −12% | 310 | −13% | 464 | −6% | 162 | −8% |
| S4A-391 | 1 | — | 21 | 274% | 48 | −28% | 127 | −14% | 288 | −19% | 409 | −17% | 182 | 4% |
| S4A-402 | 2 | — | 22 | 303% | 37 | −45% | 121 | −18% | 254 | −29% | 379 | −23% | 188 | 7% |
| S4A-411 | 5 | — | 12 | 115% | 36 | −46% | 113 | −23% | 252 | −29% | 373 | −24% | 211 | 20% |
| S4A-425 | 6 | — | 24 | 327% | 26 | −60% | 102 | −31% | 179 | −50% | 372 | −24% | 185 | 5% |
| S4A-426 | 1 | — | 27 | 392% | 49 | −26% | 100 | −33% | 248 | −30% | 406 | −17% | 177 | 1% |
| S4A-437 | 9 | — | 20 | 250% | 32 | −52% | 87 | −41% | 189 | −47% | 312 | −36% | 144 | −18% |
| S4A-443 | 2 | — | 18 | 222% | 26 | −62% | 92 | −37% | 262 | −27% | 399 | −19% | 194 | 10% |
| S4A-444 | 5 | — | 22 | 292% | 37 | −45% | 97 | −34% | 215 | −40% | 422 | −14% | 183 | 4% |
| S4A-445 | 6 | — | 19 | 236% | 42 | −36% | 111 | −25% | 205 | −43% | 313 | −36% | 153 | −13% |
| S4A-448 | 3 | — | 25 | 350% | 45 | −32% | 109 | −26% | 232 | −35% | 429 | −13% | 214 | 22% |
| S4A-458 | 8 | — | 32 | 476% | 41 | −38% | 114 | −23% | 275 | −23% | 438 | −11% | 234 | 33% |
| S4A-480 | 7 | — | 20 | 258% | 49 | −27% | 147 | −1% | 276 | −23% | 370 | −25% | 158 | −10% |

TABLE 11
Summary of field yield data

| | | Number of Edited Alleles | | | | |
|---|---|---|---|---|---|---|
| | | 3 | 4 | 5 | 6 | 7 |
| Salinas | # of Events | 2 | 2 | 4 | 10 | 44 |
| 2021-2022 | Average % Change of WT | 9.7% | −15.7% | −10.3% | 19.6% | 22.1% |
| | St Dev % Change of WT | 13.9% | 25.0% | 29.1% | 20.3% | 37.2% |
| Oxnard | # of Events | 2 | 2 | 7 | 12 | 61 |
| 2021-2022 | Average % Change of WT | −20.3% | 13.9% | −6.1% | −5.9% | −19.3% |
| | St Dev % Change of WT | 12.4% | 18.8% | 25.6% | 26.5% | 25.3% |
| Salinas | # of Events | 2 | 2 | 6 | 9 | 33 |
| 2022-2023 | Average % Change of WT | 18.5% | 18.9% | 21.1% | 20.4% | 23.4% |
| | St Dev % Change of WT | 16.1% | 3.3% | 23.3% | 9.0% | 11.6% |
| Oxnard | # of Events | 2 | 2 | 6 | 9 | 30 |
| 2022-2023 | Average % Change of WT | 42.6% | 13.6% | 30.9% | 12.2% | 26.9% |
| | St Dev % Change of WT | 66.5% | 36.3% | 27.7% | 24.1% | 23.3% |

TABLE 12
Average high and low temperatures for field trials

| Location | Year | Month | Avg High Temp (F.) | Avg Low Temp (F.) |
|---|---|---|---|---|
| Salinas | 2021 | November | 72.6 | 48.1 |
| Salinas | 2021 | December | 58.9 | 42.8 |
| Salinas | 2022 | January | 68.7 | 44.4 |
| Salinas | 2022 | February | 76.9 | 43.9 |
| Salinas | 2022 | March | 65.3 | 43.5 |
| Salinas | 2022 | April | 63.1 | 43.3 |
| Salinas | 2022 | May | 64 | 45.3 |
| Salinas | 2022 | June | 67.6 | 49.6 |
| Salinas | 2022 | July | 65.3 | 53.7 |
| Salinas | 2022 | August | 69 | 54.6 |
| Salinas | 2022 | September | 74.2 | 54.2 |
| Oxnard | 2021 | October | 74.1 | 49.8 |
| Oxnard | 2021 | November | 72.7 | 47.7 |
| Oxnard | 2021 | December | 60.9 | 42 |
| Oxnard | 2022 | January | 68.9 | 44 |
| Oxnard | 2022 | February | 71.8 | 42.8 |
| Oxnard | 2022 | March | 71.1 | 44.3 |
| Oxnard | 2022 | April | 70.5 | 47.1 |
| Oxnard | 2022 | May | 70.3 | 49 |
| Oxnard | 2022 | June | 76 | 54.9 |
| Salinas | 2022 | November | 63.8 | 40.2 |
| Salinas | 2022 | December | 58.8 | 42 |
| Salinas | 2023 | January | 58.4 | 42.8 |
| Salinas | 2023 | February | 58.4 | 38 |
| Salinas | 2023 | March | 58.6 | 42.1 |
| Salinas | 2023 | April | 61.6 | 43.6 |
| Salinas | 2023 | May | 61.4 | 50 |
| Salinas | 2023 | June | 63.4 | 52.2 |
| Salinas | 2023 | July | 65.1 | 52.6 |
| Salinas | 2023 | August | 69.4 | 55.9 |
| Salinas | 2023 | September | 68.1 | 54.7 |
| Oxnard | 2022 | October | 75.7 | 54.3 |
| Oxnard | 2022 | November | 69.4 | 44.3 |
| Oxnard | 2022 | December | 64.6 | 45.1 |
| Oxnard | 2023 | January | 62.8 | 43 |
| Oxnard | 2023 | February | 63.8 | 40.8 |
| Oxnard | 2023 | March | 61.3 | 44.4 |
| Oxnard | 2023 | April | 66.7 | 47.2 |
| Oxnard | 2023 | May | 66.6 | 52.6 |
| Oxnard | 2023 | June | 69.5 | 53.6 |

TABLE 13
Lines showing a consistent yield increase compared to wild-type

| | | | | | | | | | Salinas | Oxnard | Salinas | Oxnard |
|---|---|---|---|---|---|---|---|---|---|---|---|---|
| | | | | | | | No. of | | 2021-2022 | 2021-2022 | 2022-2023 | 2022-2023 |
| | | | | | | | Edited | | % increase | % increase | % increase | % increase |
| TFL1a | TFL1b1 | TFL1b2 | TFL1c | TFL1d1 | TFL1d2 | TFL1e | Alleles | Event | over WT | over WT | over WT | over WT |
| — | 1D | 4d 1I | 4D | 1D | 28I | 2D | 7 | S4A-031 | 43.6% | 25.9% | 20.0% | 267.9% |
| 2D | 68D | 1D | WT | — | 2D | 4D 1I | 6 | S4A-049 | 16.4% | 14.3% | 20.5% | 318.7% |
| 16D | 4D | 4D | 10D | 10D | 5D | 19D | 7 | S4A-051 | 37.5% | 4.2% | 25.1% | 153.6% |
| 4D | 24D 5I | WT | 15D 15I | 31D | 1D | 1D | 6 | S4A-115 | 17.4% | 14.0% | 25.5% | 495.5% |
| 10D | 4D | 3D | 79D | 1D | 8D | — | 7 | S4A-138 | 16.6% | 10.7% | 18.7% | 1105.8% |
| 9D | 15D | 1D | 10D | 7D | 1D | 1D | 7 | S4A-149 | 50.5% | 78.6% | 21.3% | 395.8% |
| 16D | 16D | 10D | 1D | 10D | — | 1D | 7 | S4A-165 | 25.3% | 46.7% | 33.2% | 428.9% |
| WT | 2D | 10D | 39D | 5D | 1D | 3D | 6 | S4A-168 | 33.5% | 96.1% | 28.9% | 319.6% |
| 27D | 3D | WT | — | 89D | 52I | 26D 20I | 6 | S4A-186 | 27.7% | 63.1% | 31.1% | 419.7% |
| 3D | 18D 17I | 1D | 4D 1I | 1D | 4D | 2D | 7 | S4A-210 | 12.6% | 22.9% | 38.8% | 774.0% |
| — | 1I | — | 2D | 26D | 10D | 1D | 7 | S4A-274 | 40.0% | 3.1% | 19.6% | 230.8% |
| 4D | 5D | 3D | 14D | 4D | 1D | 1D | 7 | S4A-402 | 20.9% | 44.1% | 15.1% | 220.2% |
| 4D 1I | 4D | 1D | 1D | 15D | 4D | 14D | 7 | S4A-411 | 22.5% | 8.7% | 16.7% | 559.3% |
| 21D | 9D | 1D | — | 4D | 4D | 17D | 7 | S4A-425 | 45.9% | 62.4% | 33.5% | 660.9% |
| 31D | 4D | 1D | 4D | 26D | 31D | 1D | 7 | S4A-426 | 97.2% | 33.5% | 9.4% | 187.6% |
| 1D | 2D | 2D | 1D | 4D2I | 1D | 14D | 7 | S4A-444 | 24.9% | 107.4% | 20.2% | 572.1% |
| 4D 1I | 26D | 1D | 36D 16I | 6D | 2D | 3D | 7 | S4A-445 | 54.4% | 122.4% | 38.6% | 675.3% |
| 1D | 3D | 5D | 1I | 3D | 1D | 3D 2I | 7 | S4A-448 | 139.4% | 96.4% | 48.0% | 399.9% |

As shown above, all of the lines that had a consistent increase in yield over wild-type comprised edits to both TFL1d1 and TFL1d2.

### Example 4: Strawberry Plants Generated with RNPs

Strawberry protoplast cells can also be transfected with RNP complexes consisting of purified CAS9 protein bound to a synthetic guide RNA (Andersson M., et al., (2018). Genome Editing in Potato via CRISPR-Cas9 Ribonucleoprotein Delivery. Physiologia Plantarum 164, pg. 378-384). This method does not rely on the introduction of DNA to serve as a transcriptional template, nor is an external repair template provided. Protoplasts are transfected to deliver CRISPR/Cas9 components into plant cells. As with delivery via the Agrobacterium method, the cell's natural DNA repair mechanism then repairs the break by nonhomologous end-joining, which can lead to knockout of the targeted protein function.

Protoplasts are regenerated through a callus phase into plants using regeneration medium. Plants are then grown in tissue culture where they are screened molecularly for the desired targeted genomic edits before being moved to the greenhouse or field. Because no vector DNA is used in the RNP method, plants are not molecularly screened for the absence of vector DNA.

As 4-5 mm shoot clumps form, they are cut from the calli and transferred to Magenta boxes containing FaR medium supplemented with 300 mg/L Timentin and 1 mL/L PPM. Shoot clumps are cultured with a 16-hour photoperiod at 24° C. for 28 days. After 28 days, a single shoot per shoot clump is transferred into FaR medium supplemented with 300 mg/L Timentin and 1 mL/L PPM and are cultured for an additional 28 days before leaf samples are submitted for molecular characterization and event selection.

qPCR of gRNA6 and a control site was performed and a total of 5% of events returned positive for mutations in one or more alleles of TFL1. Mutated events were recollected by transformation and DNA was isolated via the CTAB method. Events were tested by ddPCR for the target site and an external reference (AGP). A number of events were confirmed to have at least one mutation in the TFL1 gene. Mutated events were designated S4A to encompass the variety ‘Inspire’ and the remontancy trait.

DNA was extracted from 100 selected S4A events (plus WT). To prepare for sequencing, a 440 bp amplicon was barcoded (IDT xGen UDI), products were checked on a 1% agarose gel, then pooled and sent to SeqMatic. Paired-end 250 nt reads were obtained using an Illumina MiSeq nano run. After demultiplexing, paired reads were merged, primers trimmed, and sequences clustered (CD-HIT) requiring 100% identity. The read counts in the various clusters were adjusted for PCR bias towards short amplicons, and unique allele dosage was calculated. The representative cluster reference sequences (CRS) were aligned to wildtype reference sequences and used to summarize the edits (shown in Table 14 below). Plants have deletions and/or insertions in both TFL1d1 and TFL1d2 are noted with bold font.

TABLE 14
Plants edited using the RNP method

| | | | | | | | | No. of |
|---|---|---|---|---|---|---|---|---|
| | | | | | | | | Edited |
| Event | TFL1a | TFL1b1 | TFL1b2 | TFL1c | TFL1d1 | TFL1d2 | TFL1e | Alleles |
| S4A-503 | WT | WT | WT | WT | WT | WT | WT | 0 |
| S4A-504 | 1D | 9D | 1D | 1D | 1D | 1D | 4D | 7 |
| S4A-505 | 2D | 4D | 1D | 1D | 8D | — | 8D | 7 |
| S4A-506 | 1D | WT | 1D | WT | WT | 1D | WT | 3 |
| S4A-510 | 1D | WT | 1D | WT | WT | 1D | 1D | 4 |
| S4A-511 | WT | WT | WT | WT | WT | WT | WT | 0 |
| S4A-512 | WT | WT | WT | 4D | 1D | 1D | 10D | 4 |
| S4A-513 | WT | WT | WT | WT | WT | WT | WT | 0 |
| S4A-514 | WT | WT | WT | WT | WT | WT | WT | 0 |
| S4A-515 | 1D | 2D | 1D | 1D | 2D | 1D | 1D | 7 |
| S4A-516 | WT | 1D | WT | WT | WT | WT | WT | 1 |
| S4A-517 | 1D | 10D | 4D | 4D | 4D | 1D | 1D | 7 |
| S4A-518 | 10D | 2D | 1D | 1D | 4D | 1D | 1D | 7 |
| S4A-519 | 1D | 1D | 2D, 2I | 1D | 1D | 1D | 4D | 7 |
| S4A-520 | WT | WT | WT | 4D | WT | WT | 14D | 2 |
| S4A-523 | WT | WT | WT | 4D | WT | WT | 14D | 2 |
| S4A-524 | 1D | WT | 1D | WT | WT | WT | WT | 2 |
| S4A-525 | 3D | 1D | WT | 1D | WT | 1D | WT | 4 |
| S4A-527 | WT | WT | WT | 4D | WT | WT | 14D | 2 |
| S4A-537 | WT | WT | — | WT | WT | WT | WT | 1 |
| S4A-544 | 1D | WT | 1D | WT | WT | WT | WT | 2 |
| S4A-554 | WT | WT | WT | WT | WT | — | WT | 1 |
| S4A-558 | 1D | WT | 1D | WT | WT | WT | WT | 2 |
| S4A-564 | WT | WT | WT | WT | 2D | WT | WT | 1 |
| S4A-574 | WT | 1D | WT | WT | WT | WT | WT | 1 |
| S4A-590 | WT | WT | WT | WT | 1D | WT | WT | 1 |
| S4A-592 | WT | WT | WT | WT | 1D | 1D | WT | 2 |

D = deletion of the number of nucleotides indicated by the preceding number. I = insertion of the number of nucleotides indicated by the preceding number. WT = wild type. A dash indicates deletion present but not quantified.

### Example 5: Weeks to Anthesis after Planting of RNP Edited Lines

Summer planted 2023 field consisted of bareroot plants produced in a low elevation nursery located at Manteca, CA. Plants were harvested and placed into cold storage (32 F) for approximately 6 weeks. Two reps of twenty plants were hand planted into raised beds with plastic mulch in Oxnard, CA on Jul. 13, 2023. Plants were maintained according to the usual grower practices.

Fall planted 2023-2024 fields consisted of bareroot plants produced in a high elevation nursery located at Macdoel, CA. Following commercial standards, plants were harvested after 265 chill hours for Oxnard and 447 chill hours for Salinas. Two reps of twenty-five plants were hand planted into raised beds with plastic mulch in Oxnard, CA on Oct. 10, 2023, and in Salinas, CA on Oct. 31, 2023. Plants were maintained according to usual grower practices.

Plants with at least one open flower were documented at various intervals or weeks after planting (WAP) over the growing season. The number of viable plants per plot were documented to find the percent of plants per plot that had reached anthesis (Tables 15a-15c).

A complete description of edits for each allele in each event is given in Table 14 above.

TABLE 15a
Field flowering data collected in Oxnard, California in summer 2023

| | Oxnard, California 2023 | | | | | |
|---|---|---|---|---|---|---|
| | 6 Weeks | 7 Weeks | 13 Weeks | TFL1d1 | TFL1d2 | Total # of |
| Event | % flowering | % flowering | % flowering | Edit | Edit | Edited Alleles |
| WT | 8% | 10% | 22% | n/a | n/a | 0 |
| S4A-503 | 3% | 8% | 15% | WT | WT | 0 |
| S4A-504 | 68% | 100% | 100% | 1D | 1D | 7 |
| S4A-505 | 73% | 100% | 100% | 8D | — | 7 |
| S4A-506 | 23% | 58% | 60% | WT | 1D | 3 |
| S4A-510 | 23% | 33% | 58% | WT | 1D | 4 |
| S4A-511 | 0% | 0% | 30% | WT | WT | 0 |
| S4A-512 | 55% | 100% | 100% | 1D | 1D | 4 |
| S4A-513 | 0% | 0% | 50% | WT | WT | 0 |
| S4A-514 | 0% | 0% | 26% | WT | WT | 0 |
| S4A-515 | 77% | 95% | 100% | 2D | 1D | 7 |
| S4A-516 | 8% | 10% | 23% | WT | WT | 1 |
| S4A-517 | 63% | 100% | 100% | 4D | 1D | 7 |
| S4A-519 | 69% | 95% | 97% | 1D | 1D | 7 |
| S4A-520 | 0% | 0% | 48% | WT | WT | 2 |
| S4A-523 | 5% | 8% | 23% | WT | WT | 2 |
| S4A-524 | 30% | 43% | 73% | WT | WT | 2 |
| S4A-525 | 10% | 28% | 100% | WT | 1D | 4 |
| S4A-527 | 8% | 10% | 55% | WT | WT | 2 |
| S4A-537 | 18% | 18% | 18% | WT | WT | 1 |
| S4A-544 | 10% | 13% | 28% | WT | WT | 2 |
| S4A-554 | 3% | 15% | 33% | WT | — | 1 |
| S4A-558 | 3% | 5% | 10% | WT | WT | 2 |
| S4A-564 | 3% | 15% | 93% | 2D | WT | 1 |
| S4A-574 | 13% | 18% | 36% | WT | WT | 1 |
| S4A-592 | 70% | 100% | 100% | 1D | 1D | 2 |

TABLE 15b
Field flowering data collected in Oxnard, California in 2023-2024

| | Oxnard, California 2023-2024 | | | | | |
|---|---|---|---|---|---|---|
| | 3 Weeks | 8 Weeks | 13 Weeks | TFL1d1 | TFL1d2 | Total # of |
| Event | % flowering | % flowering | % flowering | Edit | Edit | Edited Alleles |
| WT | 0% | 18% | 100% | n/a | n/a | 0 |
| S4A-503 | 0% | 6% | 100% | WT | WT | 0 |
| S4A-504 | 79% | 100% | 100% | 1D | 1D | 7 |
| S4A-505 | 68% | 92% | 100% | 8D | — | 7 |
| S4A-506 | 0% | 26% | 100% | WT | 1D | 3 |
| S4A-510 | 0% | 72% | 100% | WT | 1D | 4 |
| S4A-511 | 0% | 12% | 100% | WT | WT | 0 |
| S4A-512 | 68% | 100% | 100% | 1D | 1D | 4 |
| S4A-513 | 0% | 13% | 100% | WT | WT | 0 |
| S4A-514 | 0% | 2% | 100% | WT | WT | 0 |
| S4A-515 | 64% | 100% | 100% | 2D | 1D | 7 |
| S4A-516 | 0% | 10% | 100% | WT | WT | 1 |
| S4A-517 | 15% | 19% | 33% | 4D | 1D | 7 |
| S4A-518 | 66% | 100% | 100% | 4D | 1D | 7 |
| S4A-519 | 72% | 100% | 100% | 1D | 1D | 7 |
| S4A-520 | 0% | 34% | 100% | WT | WT | 2 |
| S4A-523 | 0% | 36% | 100% | WT | WT | 2 |
| S4A-524 | 0% | 8% | 100% | WT | WT | 2 |
| S4A-525 | 0% | 6% | 100% | WT | 1D | 4 |
| S4A-527 | 0% | 22% | 100% | WT | WT | 2 |
| S4A-537 | 0% | 13% | 100% | WT | WT | 1 |
| S4A-544 | 0% | 16% | 100% | WT | WT | 2 |
| S4A-554 | 4% | 52% | 100% | WT | — | 1 |
| S4A-558 | 0% | 8% | 100% | WT | WT | 2 |
| S4A-564 | 0% | 4% | 100% | 2D | WT | 1 |
| S4A-574 | 0% | 12% | 100% | WT | WT | 1 |
| SA4-590 | 4% | 13% | 100% | 1D | WT | 1 |
| S4A-592 | 38% | 100% | 100% | 1D | 1D | 2 |

TABLE 15c
Field flowering data collected in Salinas, California in 2023-2024

| | Salinas, California 2023-2024 | | | | | | |
|---|---|---|---|---|---|---|---|
| | 3 Weeks | 8 Weeks | 13 Weeks | 14 Weeks | TFL1d1 | TFL1d2 | Total # of |
| Event | % flowering | % flowering | % flowering | % flowering | Edit | Edit | Edited Alleles |
| WT | 0% | 1% | 67% | 100% | n/a | n/a | 0 |
| S4A-503 | 0% | 0% | 73% | 98% | WT | WT | 0 |
| S4A-504 | 39% | 67% | 98% | 96% | 1D | 1D | 7 |
| S4A-505 | 38% | 74% | 100% | 100% | 8D | — | 7 |
| S4A-506 | 0% | 0% | 71% | 100% | WT | 1D | 3 |
| S4A-510 | 0% | 0% | 98% | 100% | WT | 1D | 4 |
| S4A-511 | 0% | 0% | 71% | 100% | WT | WT | 0 |
| S4A-512 | 25% | 45% | 96% | 90% | 1D | 1D | 4 |
| S4A-513 | 0% | 0% | 25% | 88% | WT | WT | 0 |
| S4A-514 | 0% | 0% | 14% | 90% | WT | WT | 0 |
| S4A-515 | 54% | 88% | 98% | 90% | 2D | 1D | 7 |
| S4A-516 | 0% | 0% | 65% | 94% | WT | WT | 1 |
| S4A-517 | 8% | 17% | 19% | 88% | 4D | 1D | 7 |
| S4A-518 | 44% | 68% | 90% | 100% | 4D | 1D | 7 |
| S4A-519 | 61% | 88% | 100% | 96% | 1D | 1D | 7 |
| S4A-520 | 0% | 0% | 50% | 92% | WT | WT | 2 |
| S4A-523 | 0% | 0% | 58% | 100% | WT | WT | 2 |
| S4A-524 | 0% | 0% | 42% | 100% | WT | WT | 2 |
| S4A-525 | 2% | 4% | 58% | 100% | WT | 1D | 4 |
| S4A-527 | 0% | 0% | 33% | 100% | WT | WT | 2 |
| S4A-537 | 0% | 0% | 38% | 100% | WT | WT | 1 |
| S4A-544 | 0% | 0% | 50% | 92% | WT | WT | 2 |
| S4A-554 | 0% | 0% | 44% | 67% | WT | — | 1 |
| S4A-558 | 0% | 0% | 54% | 100% | WT | WT | 2 |
| S4A-564 | 0% | 0% | 12% | 94% | 2D | WT | 1 |
| S4A-574 | 0% | 0% | 52% | 100% | WT | WT | 1 |
| SA4-590 | 2% | 10% | 48% | 100% | 1D | WT | 1 |
| S4A-592 | 27% | 57% | 90% | 100% | 1D | 1D | 2 |

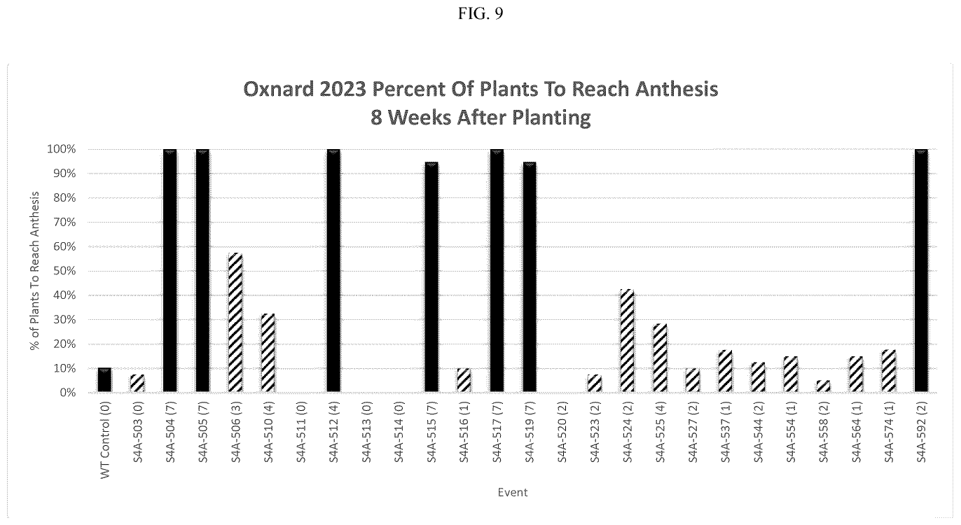

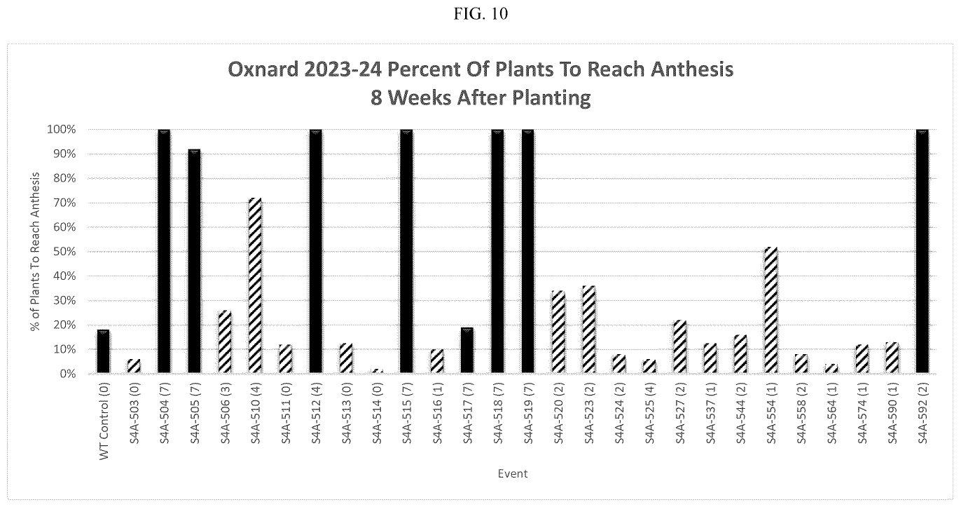

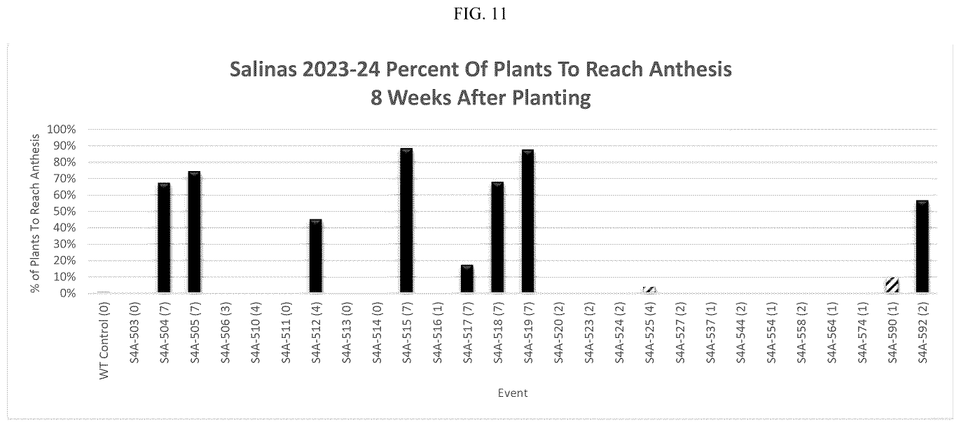

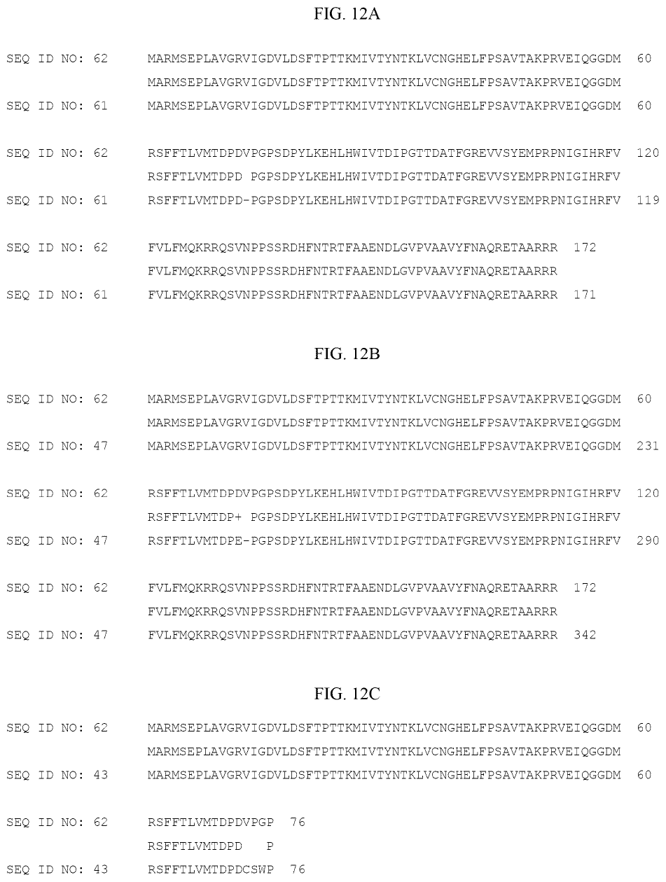

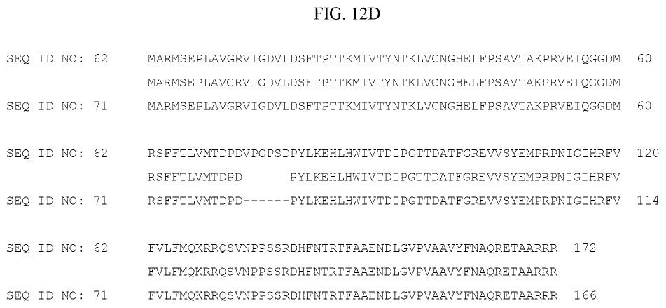

Similar to the lines generated via the Agrobacterium method, there was an inverse trend for the number of TFL1 alleles edited and weeks to flower, wherein the greater number of TFL1 alleles edited correlated with earlier weeks to flower compared to wildtype, and plants having both TFL1d1 and TFL1d2 alleles edited flower earliest (bold font in Tables 15a-15c, events SA4-504, SA4-505, SA4-512, SA4-515, SA4-505, SA4-517, SA4-518, SA4-519, and SA4-592). See also FIGS. 9-11.

### Example 6: 2023 and 2024 Yield Data of Field Trials

Plants were grown and transplanted to fields as described above in Example 3. Fruit was hand harvested twice a week and documented as marketable or cull. Total fruit was included as marketable and cull fruit and is shown in the Tables below as grams per plant (g/plant). Plant events in bold comprise mutations in both TFL1d1 and TFL1d2 alleles. A complete description of edits for each allele in each event is given in Tables 5a, 5b, and 14 above. Plant events in rows 1-41 of Table 16a were generated via Agrobacterium transformation, and plant events in rows 42-67 were generated via the RNP method. All plants had undergone vernalization or conditioning before planting.

TABLE 16a
Field yield data for Oxnard, California 2023

| | | September | October | November | December | Season |
|---|---|---|---|---|---|---|
| | Event | g/plant | g/plant | g/plant | g/plant | g/plant |
| 1 | WT | 16 | 75 | 19 | 5 | 115 |
| | Control | | | | | |
| 2 | S4A-448 | 256 | 735 | 336 | 112 | 1439 |
| 3 | S4A-391 | 210 | 694 | 393 | 132 | 1429 |
| 4 | S4A-210 | 256 | 668 | 371 | 133 | 1428 |
| 5 | S4A-307 | 198 | 621 | 407 | 129 | 1355 |
| 6 | S4A-426 | 208 | 581 | 407 | 135 | 1331 |
| 7 | S4A-444 | 278 | 602 | 327 | 112 | 1319 |
| 8 | S4A-018 | 248 | 628 | 324 | 91 | 1291 |
| 9 | S4A-048 | 250 | 616 | 318 | 105 | 1289 |
| 10 | S4A-194 | 235 | 621 | 334 | 96 | 1286 |
| 11 | S4A-402 | 204 | 566 | 382 | 133 | 1285 |
| 12 | S4A-162 | 269 | 620 | 294 | 96 | 1279 |
| 13 | S4A-443 | 212 | 603 | 347 | 93 | 1256 |
| 14 | S4A-049 | 235 | 592 | 330 | 98 | 1255 |
| 15 | S4A-358 | 220 | 580 | 336 | 119 | 1254 |
| 16 | S4A-051 | 193 | 641 | 327 | 87 | 1248 |
| 17 | S4A-437 | 260 | 572 | 306 | 101 | 1239 |
| 18 | S4A-031 | 243 | 600 | 279 | 92 | 1214 |
| 19 | S4A-186 | 203 | 591 | 329 | 91 | 1213 |
| 20 | S4A-115 | 216 | 555 | 306 | 128 | 1206 |
| 21 | S4A-154 | 233 | 611 | 274 | 79 | 1197 |
| 22 | S4A-019 | 256 | 562 | 264 | 88 | 1171 |
| 23 | S4A-445 | 246 | 540 | 280 | 104 | 1170 |
| 24 | S4A-165 | 207 | 570 | 303 | 86 | 1166 |
| 25 | S4A-209 | 196 | 523 | 336 | 110 | 1165 |
| 26 | S4A-134 | 266 | 563 | 254 | 80 | 1163 |
| 27 | S4A-214 | 228 | 540 | 306 | 75 | 1150 |
| 28 | S4A-353 | 232 | 525 | 268 | 98 | 1122 |
| 29 | S4A-480 | 245 | 558 | 216 | 95 | 1113 |
| 30 | S4A-029 | 164 | 504 | 299 | 117 | 1083 |
| 31 | S4A-180 | 225 | 533 | 228 | 80 | 1065 |
| 32 | S4A-098 | 80 | 325 | 78 | 14 | 496 |
| 33 | S4A-119 | 69 | 295 | 110 | 18 | 492 |
| 34 | S4A-336 | 65 | 238 | 101 | 22 | 426 |
| 35 | S4A-390 | 51 | 250 | 92 | 19 | 413 |
| 36 | S4A-183 | 31 | 174 | 103 | 25 | 334 |
| 37 | S4A-123 | 41 | 189 | 51 | 9 | 291 |
| 38 | S4A-378 | 39 | 158 | 57 | 9 | 263 |
| 39 | S4A-227 | 27 | 149 | 66 | 18 | 261 |
| 40 | S4A-334 | 36 | 125 | 46 | 7 | 215 |
| 41 | S4A-293 | 21 | 58 | 10 | 3 | 91 |
| 42 | S4A-512 | 244 | 617 | 316 | 134 | 1311 |
| 43 | S4A-515 | 222 | 570 | 316 | 97 | 1205 |
| 44 | S4A-592 | 235 | 624 | 243 | 94 | 1196 |
| 45 | S4A-505 | 206 | 555 | 315 | 113 | 1189 |
| 46 | S4A-519 | 218 | 435 | 177 | 58 | 887 |
| 47 | S4A-504 | 147 | 386 | 230 | 71 | 835 |
| 48 | S4A-564 | 41 | 261 | 90 | 18 | 409 |
| 49 | WT | 67 | 214 | 89 | 27 | 397 |
| | Control | | | | | |
| 50 | S4A-510 | 41 | 235 | 94 | 19 | 388 |
| 51 | S4A-506 | 86 | 223 | 53 | 12 | 374 |
| 52 | S4A-524 | 39 | 166 | 59 | 9 | 274 |
| 53 | S4A-527 | 20 | 157 | 46 | 15 | 238 |
| 54 | S4A-544 | 20 | 125 | 39 | 2 | 186 |
| 55 | S4A-523 | 16 | 114 | 48 | 4 | 182 |
| 56 | S4A-574 | 28 | 122 | 26 | 4 | 179 |
| 57 | S4A-516 | 14 | 115 | 41 | 4 | 174 |
| 58 | S4A-520 | 8 | 114 | 33 | 2 | 157 |
| 59 | S4A-517 | 30 | 107 | 17 | 3 | 156 |
| 60 | S4A-554 | 28 | 93 | 17 | 2 | 139 |
| 61 | S4A-511 | 13 | 88 | 17 | 1 | 119 |
| 62 | S4A-525 | 10 | 66 | 23 | 4 | 103 |
| 63 | S4A-513 | 0 | 67 | 0 | 0 | 68 |
| 64 | S4A-503 | 2 | 46 | 1 | 1 | 49 |
| 65 | S4A-514 | 0 | 21 | 18 | 6 | 46 |
| 66 | S4A-558 | 3 | 24 | 6 | 1 | 33 |
| 67 | S4A-537 | 6 | 26 | 0 | 0 | 32 |

TABLE 16b
Field yield data for Oxnard, California 2023-2024

| | December | January | February | March | April | May | June | |
|---|---|---|---|---|---|---|---|---|
| | 2023 | 2024 | 2024 | 2024 | 2024 | 2024 | 2024 | Season |
| | g/plant | g/plant | g/plant | g/plant | g/plant | g/plant | g/plant | g/plant |
| WT Control | 0 | 65 | 131 | 319 | 460 | 412 | 250 | 1637 |
| S4A-336 | 2 | 86 | 167 | 359 | 455 | 351 | 214 | 1636 |
| S4A-293 | 0 | 60 | 124 | 326 | 475 | 370 | 206 | 1560 |
| S4A-334 | 1 | 89 | 168 | 342 | 429 | 316 | 201 | 1546 |
| S4A-448 | 13 | 32 | 77 | 205 | 337 | 484 | 318 | 1465 |
| S4A-444 | 14 | 41 | 66 | 189 | 294 | 419 | 284 | 1307 |
| S4A-480 | 10 | 38 | 82 | 254 | 296 | 345 | 266 | 1292 |
| S4A-115 | 12 | 36 | 45 | 162 | 266 | 385 | 278 | 1184 |
| S4A-210 | 17 | 39 | 29 | 159 | 252 | 377 | 255 | 1129 |
| S4A-520 | 1 | 87 | 148 | 348 | 504 | 399 | 207 | 1693 |
| S4A-523 | 0 | 63 | 122 | 351 | 480 | 392 | 212 | 1620 |
| S4A-516 | 5 | 69 | 134 | 302 | 456 | 423 | 201 | 1590 |
| S4A-527 | 9 | 57 | 126 | 304 | 491 | 386 | 213 | 1586 |
| S4A-511 | 0 | 63 | 151 | 293 | 453 | 416 | 205 | 1582 |
| S4A-544 | 0 | 66 | 124 | 283 | 430 | 422 | 257 | 1582 |
| S4A-514 | 0 | 14 | 74 | 186 | 500 | 561 | 235 | 1570 |
| S4A-558 | 0 | 30 | 83 | 292 | 501 | 425 | 234 | 1565 |
| S4A-574 | 0 | 44 | 109 | 295 | 432 | 428 | 247 | 1556 |
| S4A-554 | 2 | 78 | 127 | 227 | 349 | 416 | 263 | 1462 |
| S4A-592 | 11 | 45 | 82 | 216 | 362 | 435 | 264 | 1415 |
| S4A-506 | 0 | 40 | 72 | 198 | 309 | 417 | 283 | 1320 |
| S4A-512 | 11 | 32 | 38 | 163 | 294 | 446 | 324 | 1308 |
| S4A-503 | 0 | 41 | 72 | 279 | 420 | 368 | 125 | 1304 |
| S4A-505 | 11 | 34 | 60 | 163 | 299 | 424 | 273 | 1263 |
| S4A-524 | 0 | 45 | 99 | 211 | 346 | 376 | 176 | 1253 |
| S4A-515 | 11 | 21 | 43 | 147 | 233 | 441 | 332 | 1228 |
| S4A-564 | 0 | 32 | 78 | 187 | 336 | 386 | 207 | 1225 |
| S4A-504 | 12 | 24 | 21 | 104 | 246 | 431 | 249 | 1086 |
| S4A-518 | 3 | 24 | 46 | 139 | 226 | 364 | 272 | 1073 |
| S4A-519 | 14 | 26 | 19 | 185 | 258 | 299 | 256 | 1056 |
| S4A-513 | 0 | 23 | 58 | 164 | 282 | 364 | 162 | 1054 |
| S4A-510 | 1 | 84 | 75 | 171 | 240 | 237 | 138 | 947 |
| S4A-537 | 0 | 36 | 29 | 138 | 199 | 326 | 182 | 911 |
| S4A-525 | 0 | 3 | 14 | 45 | 93 | 89 | 34 | 279 |
| S4A-590 | 0 | 8 | 9 | 23 | 70 | 64 | 15 | 189 |
| S4A-517 | 2 | 0 | 0 | 0 | 28 | 3 | 0 | 33 |

TABLE 16c
Field yield data for Oxnard, California 2024

| | Sept | Oct | Nov | Dec | Season |
|---|---|---|---|---|---|
| | g/plant | g/plant | g/plant | g/plant | g/plant |
| S4A-448 | 110 | 509 | 389 | 287 | 1295 |
| S4A-391 | 104 | 439 | 348 | 285 | 1176 |
| S4A-210 | 98 | 448 | 345 | 253 | 1144 |
| S4A-444 | 86 | 405 | 311 | 231 | 1034 |
| S4A-115 | 96 | 413 | 309 | 210 | 1028 |
| S4A-480 | 96 | 429 | 313 | 178 | 1015 |
| S4A-334 | 42 | 119 | 85 | 45 | 291 |
| S4A-336 | 32 | 105 | 57 | 31 | 225 |
| S4A-293 | 11 | 12 | 9 | 8 | 40 |

TABLE 17
Field yield data for Salinas, California 2024

| | April | May | June | July | August | September | Season |
|---|---|---|---|---|---|---|---|
| | g/ | g/ | g/ | g/ | g/ | g/ | g/ |
| | plant | plant | plant | plant | plant | plant | plant |
| S4A-444 | 191 | 481 | 646 | 572 | 577 | 732 | 3199 |
| S4A-210 | 156 | 457 | 703 | 652 | 562 | 663 | 3193 |
| S4A-448 | 145 | 540 | 709 | 556 | 538 | 658 | 3146 |
| S4A-115 | 161 | 420 | 620 | 622 | 602 | 715 | 3140 |
| S4A-336 | 293 | 623 | 568 | 476 | 490 | 485 | 2934 |
| S4A-334 | 330 | 638 | 487 | 462 | 557 | 379 | 2851 |
| S4A-293 | 321 | 687 | 566 | 452 | 435 | 351 | 2812 |
| S4A-480 | 183 | 462 | 557 | 528 | 476 | 493 | 2698 |
| WT | 223 | 585 | 576 | 428 | 374 | 412 | 2599 |
| Control | | | | | | | |

### NUMBERED EMBODIMENTS

1. A cultivated Rosaceae plant, plant part, or plant cell having genetically engineered Terminal Flowering d1 and d2 alleles (TFL1d1 and TFL1d2), or homologs thereof, wherein each TFL1d allele has one or more edits that reduce or knockout protein function.
2. The cultivated Rosaceae plant, plant part, or plant cell of embodiment 1, wherein said plant further comprises one or more edits in a TFL1a, TFL1b1, TFL1b2, TFL1c, or TFL1e allele, or homologs thereof, that reduce or knockout protein function.
3. The cultivated Rosaceae plant of embodiment 1 or 2, wherein the plant flowers earlier than a cultivated Rosaceae plant of the same variety having wildtype TFL1d1 and TFL1d2 alleles when grown under the same environmental conditions.
4. The cultivated Rosaceae plant of embodiment 3, wherein the plant flowers at least one week earlier than a cultivated Rosaceae plant of the same variety having wildtype TFL1d1 and TFL1d2 alleles when grown under the same environmental conditions.
5. The cultivated Rosaceae plant of embodiment 3, wherein the plant flowers at least two weeks earlier than a cultivated Rosaceae plant of the same variety having wildtype TFL1d1 and TFL1d2 alleles when grown under the same environmental conditions.
6. The cultivated Rosaceae plant of embodiment 3, wherein the plant flowers at least four weeks earlier than a cultivated Rosaceae plant of the same variety having wildtype TFL1d1 and TFL1d2 alleles when grown under the same environmental conditions.
7. The cultivated Rosaceae plant of embodiment 3, wherein the plant flowers at least six weeks earlier than a cultivated Rosaceae plant of the same variety having wildtype TFL1d1 and TFL1d2 alleles when grown under the same environmental conditions.
8. The cultivated Rosaceae plant of embodiment 3, wherein the plant flowers between six and 12 weeks earlier than a cultivated Rosaceae plant of the same variety having wildtype TFL1d1 and TFL1d2 alleles when grown under the same environmental conditions.
9. The cultivated Rosaceae plant of embodiment 3, wherein the plant flowers between 12 and 20 weeks earlier than a cultivated Rosaceae plant of the same variety having wildtype TFL1d1 and TFL1d2 alleles when grown under the same environmental conditions.
10. The cultivated Rosaceae plant of embodiment 3, wherein the plant flowers between 15 and 19 weeks earlier than a cultivated Rosaceae plant of the same variety having wildtype TFL1d1 and TFL1d2 alleles when grown under the same environmental conditions.
11. The cultivated Rosaceae plant of any one of embodiments 1-10, wherein the plant has increased yield compared to a cultivated Rosaceae plant of the same variety having wildtype TFL1d1 and TFL1d2 alleles when grown under the same environmental conditions.
12. The cultivated Rosaceae plant of embodiment 11, wherein the plant has between 1% and 10% increase in yield compared to a cultivated Rosaceae plant of the same variety having wildtype TFL1d1 and TFL1d2 alleles when grown under the same environmental conditions.
13. The cultivated Rosaceae plant of embodiment 11, wherein the plant has between 10% and 25% increase in yield compared to a cultivated Rosaceae plant of the same variety having wildtype TFL1d1 and TFL1d2 alleles when grown under the same environmental conditions.
14. The cultivated Rosaceae plant of embodiment 11, wherein the plant has between 25% and 50% increase in yield compared to a cultivated Rosaceae plant of the same variety having wildtype TFL1d1 and TFL1d2 alleles when grown under the same environmental conditions.
15. The cultivated Rosaceae plant of embodiment 11, wherein the plant has between 50% and 100% increase in yield compared to a cultivated Rosaceae plant of the same variety having wildtype TFL1d1 and TFL1d2 alleles when grown under the same environmental conditions.
16. The cultivated Rosaceae plant of embodiment 11, wherein the plant has between 100% and 500% increase in yield compared to a cultivated Rosaceae plant of the same variety having wildtype TFL1d1 and TFL1d2 alleles when grown under the same environmental conditions.
17. The cultivated Rosaceae plant, plant part, or plant cell of any one of embodiments 1-16, wherein the plant, plant part, or plant cell is a species of Fragaria.
18. The cultivated Rosaceae plant, plant part, or plant cell of embodiment 17, wherein the plant, plant part, or plant cell is selected from F. iinumae, F. nipponica, F. pentaphylla, F. vesca, F. viridis, F. moupinensis, F. orientalis, F. moschata, F. chiloensis, F. iturupensis, F. virginiana, F. cascadensis, F. x ananassa, and hybrids thereof.
19. The cultivated Rosaceae plant, plant part, or plant cell of embodiment 17, wherein the plant, plant part, or plant cell is selected from F. x ananassa, F. x bringhurstii, and F. x vescana.
20. The cultivated Rosaceae plant, plant part, or plant cell, plant, plant part, or plant cell part, or plant, plant part, or plant cell of any one of embodiments 1-16, wherein the plant, plant part, or plant cell is a species of Rubus.
21. The cultivated Rosaceae plant, plant part, or plant cell, plant, plant part, or plant cell part, or plant, plant part, or plant cell of embodiment 20, wherein the plant, plant part, or plant cell is selected from R. idaeus, R. allegheniensis, R. occidentalis, R. argutus, R. ursinus, R. laciniatus, R. ulmifolius, R. leucodermis, R. strigosus, R. ellipticus, R. subsp. rubus, and hybrids thereof.
22. The cultivated Rosaceae plant, plant part, or plant cell of any one of embodiments 1-16, wherein the plant, plant part, or plant cell is a species of Vaccinium.
23. The cultivated Rosaceae plant, plant part, or plant cell of embodiment 22, wherein the plant, plant part, or plant cell is selected from V. corymbosum, V. darrowii, V. angustifolium, V. ashei, and hybrids thereof.
24. The cultivated Rosaceae plant, plant part, or plant cell of any one of embodiments 1-16, wherein the plant, plant part, or plant cell is a species of Malus.
25. The cultivated Rosaceae plant, plant part, or plant cell of embodiment 24, wherein the plant, plant part, or plant cell is selected from M. domestica, and hybrids thereof.
26. The cultivated Rosaceae plant, plant part, or plant cell of any one of embodiments 1-26, wherein an unedited TFL1d wild-type allele shares between 70% and 100% sequence identity with SEQ ID NO: 62.
27. The cultivated Rosaceae plant, plant part, or plant cell of any one of embodiments 1-26, wherein an unedited TFL1d wild-type allele shares between 75% and 100% sequence identity with SEQ ID NO: 62.
28. The cultivated Rosaceae plant, plant part, or plant cell of any one of embodiments 1-26, wherein an unedited TFL1d wild-type allele shares between 80% and 100% sequence identity with SEQ ID NO: 62.
29. The cultivated Rosaceae plant, plant part, or plant cell of any one of embodiments 1-26, wherein an unedited TFL1d wild-type allele shares between 85% and 100% sequence identity with SEQ ID NO: 62.
30. The cultivated Rosaceae plant, plant part, or plant cell of any one of embodiments 1-26, wherein an unedited TFL1d wild-type allele shares between 86% and 100% sequence identity with SEQ ID NO: 62.
31. The cultivated Rosaceae plant, plant part, or plant cell of any one of embodiments 1-26, wherein an unedited TFL1d wild-type allele shares between 87% and 100% sequence identity with SEQ ID NO: 62.
32. The cultivated Rosaceae plant, plant part, or plant cell of any one of embodiments 1-26, wherein an unedited TFL1d wild-type allele shares between 88% and 100% sequence identity with SEQ ID NO: 62.
33. The cultivated Rosaceae plant, plant part, or plant cell of any one of embodiments 1-26, wherein an unedited TFL1d wild-type allele shares between 89% and 100% sequence identity with SEQ ID NO: 62.
34. The cultivated Rosaceae plant, plant part, or plant cell of any one of embodiments 1-26, wherein an unedited TFL1d wild-type allele shares between 90% and 100% sequence identity with SEQ ID NO: 62.
35. The cultivated Rosaceae plant, plant part, or plant cell of any one of embodiments 1-26, wherein an unedited TFL1d wild-type allele shares between 91% and 100% sequence identity with SEQ ID NO: 62.
36. The cultivated Rosaceae plant, plant part, or plant cell of any one of embodiments 1-26, wherein an unedited TFL1d wild-type allele shares between 92% and 100% sequence identity with SEQ ID NO: 62.
37. The cultivated Rosaceae plant, plant part, or plant cell of any one of embodiments 1-26, wherein an unedited TFL1d wild-type allele shares between 93% and 100% sequence identity with SEQ ID NO: 62.
38. The cultivated Rosaceae plant, plant part, or plant cell of any one of embodiments 1-26, wherein an unedited TFL1d wild-type allele shares between 94% and 100% sequence identity with SEQ ID NO: 62.
39. The cultivated Rosaceae plant, plant part, or plant cell of any one of embodiments 1-26, wherein an unedited TFL1d wild-type allele shares between 95% and 100% sequence identity with SEQ ID NO: 62.
40. The cultivated Rosaceae plant, plant part, or plant cell of any one of embodiments 1-26, wherein an unedited TFL1d wild-type allele shares between 96% and 100% sequence identity with SEQ ID NO: 62.
41. The cultivated Rosaceae plant, plant part, or plant cell of any one of embodiments 1-26, wherein an unedited TFL1d wild-type allele shares between 97% and 100% sequence identity with SEQ ID NO: 62.
42. The cultivated Rosaceae plant, plant part, or plant cell of any one of embodiments 1-26, wherein an unedited TFL1d wild-type allele shares between 98% and 100% sequence identity with SEQ ID NO: 62.
43. The cultivated Rosaceae plant, plant part, or plant cell of any one of embodiments 1-26, wherein an unedited TFL1d wild-type allele shares between 99% and 100% sequence identity with SEQ ID NO: 62.
44. The cultivated Rosaceae plant, plant part, or plant cell of any one of embodiments 1-26, wherein an unedited TFL1d wild-type allele shares between 99.1% and 100% sequence identity with SEQ ID NO: 62.
45. The cultivated Rosaceae plant, plant part, or plant cell of any one of embodiments 1-26, wherein an unedited TFL1d wild-type allele shares between 99.2% and 100% sequence identity with SEQ ID NO: 62.
46. The cultivated Rosaceae plant, plant part, or plant cell of any one of embodiments 1-26, wherein an unedited TFL1d wild-type allele shares between 99.3% and 100% sequence identity with SEQ ID NO: 62.
47. The cultivated Rosaceae plant, plant part, or plant cell of any one of embodiments 1-26, wherein an unedited TFL1d wild-type allele shares between 99.4% and 100% sequence identity with SEQ ID NO: 62.
48. The cultivated Rosaceae plant, plant part, or plant cell of any one of embodiments 1-26, wherein an unedited TFL1d wild-type allele shares between 99.5% and 100% sequence identity with SEQ ID NO: 62.
49. The cultivated Rosaceae plant, plant part, or plant cell of any one of embodiments 1-26, wherein an unedited TFL1d wild-type allele shares between 99.6% and 100% sequence identity with SEQ ID NO: 62.
50. The cultivated Rosaceae plant, plant part, or plant cell of any one of embodiments 1-26, wherein an unedited TFL1d wild-type allele shares between 99.7% and 100% sequence identity with SEQ ID NO: 62.
51. The cultivated Rosaceae plant, plant part, or plant cell of any one of embodiments 1-26, wherein an unedited TFL1d wild-type allele shares between 99.8% and 100% sequence identity with SEQ ID NO: 62.
52. The cultivated Rosaceae plant, plant part, or plant cell of any one of embodiments 1-26, wherein an unedited TFL1d wild-type allele shares between 99.9% and 100% sequence identity with SEQ ID NO: 62.
52.1 The cultivated Rosaceae plant, plant part, or plant cell of any one of embodiments 1-52, wherein the genetically engineered TFL1d1 and TFL1d2 alleles comprise one or more edits in exon 2.
52.2 The cultivated Rosaceae plant, plant part, or plant cell of any one of embodiments 1-52.1, wherein the genetically engineered TFL1d1 and TFL1d2 alleles comprise one or more edits that disrupt TFL protein interaction with a 14-3-3 protein.
52.3 The cultivated Rosaceae plant, plant part, or plant cell of any one of embodiments 1-52.2, wherein the genetically engineered TFL1d1 and TFL1d2 alleles comprise one or more edits that disrupt the TFL1 proteins substrate binding.
52.4 The cultivated Rosaceae plant, plant part, or plant cell of any one of embodiments 1-52.3, wherein the genetically engineered TFL1d1 and TFL1d2 alleles comprise an insertion in exon 2.
52.5 The cultivated Rosaceae plant, plant part, or plant cell of any one of embodiments 1-52.4, wherein the genetically engineered TFL1d1 and TFL1d2 alleles comprise a deletion in exon 2.
52.6 The cultivated Rosaceae plant, plant part, or plant cell of any one of embodiments 1-52.5, wherein the genetically engineered TFL1d1 and TFL1d2 alleles comprise a frameshift mutation.
52.7 The cultivated Rosaceae plant, plant part, or plant cell of any one of embodiments 1-52.6, wherein the genetically engineered TFL1d1 and TFL1d2 alleles comprise an early stop codon.
52.8 The cultivated Rosaceae plant, plant part, or plant cell of any one of embodiments 1-52.7, wherein the genetically engineered TFL1d1 and TFL1d2 alleles comprise a complete deletion of exon 2.
52.9 The cultivated Rosaceae plant, plant part, or plant cell of any one of embodiments 1-52.7, wherein the genetically engineered TFL1d1 and TFL1d2 alleles comprise a combination of insertions and deletions in exon 2.
53. A cultivated Fragaria sp. plant, plant part, or plant cell having an early flowering trait, wherein said early flowering trait is caused by genetically engineered Terminal Flowering d1 and d2 alleles (TFL1d1 and TFL1d2), and wherein each TFL1d allele has one or more edits that reduce or knockout protein function.
54. A cultivated Fragaria sp. plant, plant part, or plant cell having an early flowering trait, wherein said early flowering trait is caused by genetically engineered Terminal Flowering d1 and d2 alleles (TFL1d1 and TFL1d2), and wherein each TFL1d allele has one or more edits in exon 2.
55. A cultivated Fragaria sp. plant, plant part, or plant cell having an early flowering trait, wherein said early flowering trait is caused by genetically engineered Terminal Flowering d1 and d2 alleles (TFL1d1 and TFL1d2), and wherein each TFL1d allele has one or more edits that disrupt TFL protein interaction with a 14-3-3 protein.
56. A cultivated Fragaria sp. plant, plant part, or plant cell having an early flowering trait, wherein said early flowering trait is caused by genetically engineered Terminal Flowering d1 and d2 alleles (TFL1d1 and TFL1d2), and wherein each TFL1d allele has one or more edits that disrupt the TFL proteins substrate binding.
57. The cultivated Fragaria sp. plant, plant part, or plant cell of any one of embodiments 53-56, wherein the one or more edits comprise an insertion in exon 2.
58. The cultivated Fragaria sp. plant, plant part, or plant cell of any one of embodiments 53-56, wherein the one or more edits comprise a deletion in exon 2.
59. The cultivated Fragaria sp. plant, plant part, or plant cell of any one of embodiments 53-56, wherein the one or more edits result in a frameshift mutation.
60. The cultivated Fragaria sp. plant, plant part, or plant cell of any one of embodiments 53-56, wherein the one or more edits result in an early stop codon.
61. The cultivated Fragaria sp. plant, plant part, or plant cell of any one of embodiments 53-56, wherein the one or more edits comprise a complete deletion of exon 2.
62. The cultivated Fragaria sp. plant, plant part, or plant cell of any one of embodiments 53-56, wherein the one or more edits to each TFL1d allele comprise a combination of insertions and deletions in exon 2.
63. The cultivated Fragaria sp. plant, plant part, or plant cell of embodiment 53, wherein the engineered TFL1d alleles comprise at least one sequence selected from SEQ ID NOs: 1-20, SEQ ID NOs: 22-41, or sequences at least 75% identical thereto.
64. The cultivated Fragaria sp. plant, plant part, or plant cell of embodiment 53, wherein the engineered TFL1d alleles comprise at least one sequence selected from SEQ ID NOs: 1-20, SEQ ID NOs: 22-41, or sequences at least 80% identical thereto.
65. The cultivated Fragaria sp. plant, plant part, or plant cell of embodiment 53, wherein the engineered TFL1d alleles comprise at least one sequence selected from SEQ ID NOs: 1-20, SEQ ID NOs: 22-41, or sequences at least 85% identical thereto.
66. The cultivated Fragaria sp. plant, plant part, or plant cell of embodiment 53, wherein the engineered TFL1d alleles comprise at least one sequence selected from SEQ ID NOs: 1-20, SEQ ID NOs: 22-41, or sequences at least 90% identical thereto.
67. The cultivated Fragaria sp. plant, plant part, or plant cell of embodiment 53, wherein the engineered TFL1d alleles comprise at least one sequence selected from SEQ ID NOs: 1-20, SEQ ID NOs: 22-41, or sequences at least 95% identical thereto.
68. The cultivated Fragaria sp. plant, plant part, or plant cell of embodiment 63, wherein the engineered TFL1d alleles comprise SEQ ID NOs: 1-2, or sequences at least 75% identical thereto.
69. The cultivated Fragaria sp. plant, plant part, or plant cell of embodiment 63, wherein the engineered TFL1d alleles comprise SEQ ID NOs: 3-4, or sequences at least 75% identical thereto.
70. The cultivated Fragaria sp. plant, plant part, or plant cell of embodiment 63, wherein the engineered TFL1d alleles comprise SEQ ID NOs: 5-6, or sequences at least 75% identical thereto.
71. The cultivated Fragaria sp. plant, plant part, or plant cell of embodiment 63, wherein the engineered TFL1d alleles comprise SEQ ID NOs: 7-8, or sequences at least 75% identical thereto.
72. The cultivated Fragaria sp. plant, plant part, or plant cell of embodiment 63, wherein the engineered TFL1d alleles comprise SEQ ID NOs: 9-10, or sequences at least 75% identical thereto.
73. The cultivated Fragaria sp. plant, plant part, or plant cell of embodiment 63, wherein the engineered TFL1d alleles comprise SEQ ID NOs: 11-12, or sequences at least 75% identical thereto.
74. The cultivated Fragaria sp. plant, plant part, or plant cell of embodiment 63, wherein the engineered TFL1d alleles comprise SEQ ID NOs: 13-14, or sequences at least 75% identical thereto.
75. The cultivated Fragaria sp. plant, plant part, or plant cell of embodiment 63, wherein the engineered TFL1d alleles comprise SEQ ID NOs: 15-16, or sequences at least 75% identical thereto.
76. The cultivated Fragaria sp. plant, plant part, or plant cell of embodiment 63, wherein the engineered TFL1d alleles comprise SEQ ID NOs: 17-18, or sequences at least 75% identical thereto.
77. The cultivated Fragaria sp. plant, plant part, or plant cell of embodiment 63, wherein the engineered TFL1d alleles comprise SEQ ID NOs: 19-20, or sequences at least 75% identical thereto.
78. The cultivated Fragaria sp. plant, plant part, or plant cell of embodiment 63, wherein the engineered TFL1d alleles comprise SEQ ID NOs: 22-23, or sequences at least 75% identical thereto.
79. The cultivated Fragaria sp. plant, plant part, or plant cell of embodiment 63, wherein the engineered TFL1d alleles comprise SEQ ID NOs: 24-25, or sequences at least 75% identical thereto.
80. The cultivated Fragaria sp. plant, plant part, or plant cell of embodiment 63, wherein the engineered TFL1d alleles comprise SEQ ID NOs: 26-27, or sequences at least 75% identical thereto.
81. The cultivated Fragaria sp. plant, plant part, or plant cell of embodiment 63, wherein the engineered TFL1d alleles comprise SEQ ID NOs: 28-29, or sequences at least 75% identical thereto.
82. The cultivated Fragaria sp. plant, plant part, or plant cell of embodiment 63, wherein the engineered TFL1d alleles comprise SEQ ID NOs: 30-31, or sequences at least 75% identical thereto.
83. The cultivated Fragaria sp. plant, plant part, or plant cell of embodiment 63, wherein the engineered TFL1d alleles comprise SEQ ID NOs: 32-33, or sequences at least 75% identical thereto.
84. The cultivated Fragaria sp. plant, plant part, or plant cell of embodiment 63, wherein the engineered TFL1d alleles comprise SEQ ID NOs: 34-35, or sequences at least 75% identical thereto.
85. The cultivated Fragaria sp. plant, plant part, or plant cell of embodiment 63, wherein the engineered TFL1d alleles comprise SEQ ID NOs: 36-37, or sequences at least 75% identical thereto.
86. The cultivated Fragaria sp. plant, plant part, or plant cell of embodiment 63, wherein the engineered TFL1d alleles comprise SEQ ID NOs: 38-39, or sequences at least 75% identical thereto.
87. The cultivated Fragaria sp. plant, plant part, or plant cell of embodiment 63, wherein the engineered TFL1d alleles comprise SEQ ID NOs: 40-41, or sequences at least 75% identical thereto.
88. The cultivated Fragaria sp. plant, plant part, or plant cell of embodiment 53, wherein the engineered TFL1d alleles encode a protein sequence selected from SEQ ID NOs: 42-61, SEQ ID NOs: 63-82, or sequences at least 75% identical thereto.
89. The cultivated Fragaria sp. plant, plant part, or plant cell of embodiment 53, wherein the engineered TFL1d alleles encode a protein sequence selected from SEQ ID NOs: 42-61, SEQ ID NOs: 63-82, or sequences at least 80% identical thereto.
90. The cultivated Fragaria sp. plant, plant part, or plant cell of embodiment 53, wherein the engineered TFL1d alleles encode a protein sequence selected from SEQ ID NOs: 42-61, SEQ ID NOs: 63-82, or sequences at least 85% identical thereto.
91. The cultivated Fragaria sp. plant, plant part, or plant cell of embodiment 53, wherein the engineered TFL1d alleles encode a protein sequence selected from SEQ ID NOs: 42-61, SEQ ID NOs: 63-82, or sequences at least 90% identical thereto.
92. The cultivated Fragaria sp. plant, plant part, or plant cell of embodiment 53, wherein the engineered TFL1d alleles encode a protein sequence selected from SEQ ID NOs: 42-61, SEQ ID NOs: 63-82, or sequences at least 95% identical thereto.
93. The cultivated Fragaria sp. plant, plant part, or plant cell of embodiment 88, wherein the engineered TFL1d alleles encode SEQ ID NOs: 42-43, or sequences at least 75% identical thereto.
94. The cultivated Fragaria sp. plant, plant part, or plant cell of embodiment 88, wherein the engineered TFL1d alleles encode SEQ ID NOs: 44-45, or sequences at least 75% identical thereto.
95. The cultivated Fragaria sp. plant, plant part, or plant cell of embodiment 88, wherein the engineered TFL1d alleles encode SEQ ID NOs: 46-47, or sequences at least 75% identical thereto.
96. The cultivated Fragaria sp. plant, plant part, or plant cell of embodiment 88, wherein the engineered TFL1d alleles encode SEQ ID NOs: 48-49, or sequences at least 75% identical thereto.
97. The cultivated Fragaria sp. plant, plant part, or plant cell of embodiment 88, wherein the engineered TFL1d alleles encode SEQ ID NOs: 50-51, or sequences at least 75% identical thereto.
98. The cultivated Fragaria sp. plant, plant part, or plant cell of embodiment 88, wherein the engineered TFL1d alleles encode SEQ ID NOs: 52-53, or sequences at least 75% identical thereto.
99. The cultivated Fragaria sp. plant, plant part, or plant cell of embodiment 88, wherein the engineered TFL1d alleles encode SEQ ID NOs: 54-55, or sequences at least 75% identical thereto.
100. The cultivated Fragaria sp. plant, plant part, or plant cell of embodiment 88, wherein the engineered TFL1d alleles encode SEQ ID NOs: 56-57, or sequences at least 75% identical thereto.
101. The cultivated Fragaria sp. plant, plant part, or plant cell of embodiment 88, wherein the engineered TFL1d alleles encode SEQ ID NOs: 58-59, or sequences at least 75% identical thereto.
102. The cultivated Fragaria sp. plant, plant part, or plant cell of embodiment 88, wherein the engineered TFL1d alleles encode SEQ ID NOs: 60-61, or sequences at least 75% identical thereto.
103. The cultivated Fragaria sp. plant, plant part, or plant cell of embodiment 88, wherein the engineered TFL1d alleles encode SEQ ID NOs: 63-64, or sequences at least 75% identical thereto.
104. The cultivated Fragaria sp. plant, plant part, or plant cell of embodiment 88, wherein the engineered TFL1d alleles encode SEQ ID NOs: 65-66, or sequences at least 75% identical thereto.
105. The cultivated Fragaria sp. plant, plant part, or plant cell of embodiment 88, wherein the engineered TFL1d alleles encode SEQ ID NOs: 67-68, or sequences at least 75% identical thereto.
106. The cultivated Fragaria sp. plant, plant part, or plant cell of embodiment 88, wherein the engineered TFL1d alleles encode SEQ ID NOs: 69-70, or sequences at least 75% identical thereto.
107. The cultivated Fragaria sp. plant, plant part, or plant cell of embodiment 88, wherein the engineered TFL1d alleles encode SEQ ID NOs: 71-72, or sequences at least 75% identical thereto.
108. The cultivated Fragaria sp. plant, plant part, or plant cell of embodiment 88, wherein the engineered TFL1d alleles encode SEQ ID NOs: 73-74, or sequences at least 75% identical thereto.
109. The cultivated Fragaria sp. plant, plant part, or plant cell of embodiment 88, wherein the engineered TFL1d alleles encode SEQ ID NOs: 75-76, or sequences at least 75% identical thereto.
110. The cultivated Fragaria sp. plant, plant part, or plant cell of embodiment 88, wherein the engineered TFL1d alleles encode SEQ ID NOs: 77-78, or sequences at least 75% identical thereto.
111. The cultivated Fragaria sp. plant, plant part, or plant cell of embodiment 88, wherein the engineered TFL1d alleles encode SEQ ID NOs: 79-80, or sequences at least 75% identical thereto.
112. The cultivated Fragaria sp. plant, plant part, or plant cell of embodiment 88, wherein the engineered TFL1d alleles encode SEQ ID NOs: 81-82, or sequences at least 75% identical thereto.
113. The cultivated Fragaria sp. plant, plant part, or plant cell of any one of embodiments 53-112, wherein the plant, plant part, or plant cell further comprises one or more edits in a TFL1a, TFL1b1, TFL1b2, TFL1c, and/or TFL1e allele that reduce protein function.
114. The cultivated Fragaria sp. plant, plant part, or plant cell of embodiment 113, wherein said plant, plant part, or plant cell comprises TFL1d1, TFL1d2, and TFL1a edited alleles.
115. The cultivated Fragaria sp. plant, plant part, or plant cell of embodiment 113, wherein said plant, plant part, or plant cell comprises TFL1d1, TFL1d2, and TFL1b1 edited alleles.
116. The cultivated Fragaria sp. plant, plant part, or plant cell of embodiment 113, wherein said plant, plant part, or plant cell comprises TFL1d1, TFL1d2, and TFL1b2 edited alleles.
117. The cultivated Fragaria sp. plant, plant part, or plant cell of embodiment 113, wherein said plant, plant part, or plant cell comprises TFL1d1, TFL1d2, and TFL1c edited alleles.
118. The cultivated Fragaria sp. plant, plant part, or plant cell of embodiment 113, wherein said plant, plant part, or plant cell comprises TFL1d1, TFL1d2, and TFL1e edited alleles.
119. The cultivated Fragaria sp. plant, plant part, or plant cell of embodiment 113, wherein said plant, plant part, or plant cell comprises TFL1d1, TFL1d2, TFL1a, and TFL1b1 edited alleles.
120. The cultivated Fragaria sp. plant, plant part, or plant cell of embodiment 113, wherein said plant, plant part, or plant cell comprises TFL1d1, TFL1d2, TFL1a, and TFL1b2 edited alleles.
121. The cultivated Fragaria sp. plant, plant part, or plant cell of embodiment 113, wherein said plant, plant part, or plant cell comprises TFL1d1, TFL1d2, TFL1a, and TFL1c edited alleles.
122. The cultivated Fragaria sp. plant, plant part, or plant cell of embodiment 113, wherein said plant, plant part, or plant cell comprises TFL1d1, TFL1d2, TFL1a, and TFL1e edited alleles.
123. The cultivated Fragaria sp. plant, plant part, or plant cell of embodiment 113, wherein said plant, plant part, or plant cell comprises TFL1d1, TFL1d2, TFL1b1, and TFL1b2 edited alleles.
124. The cultivated Fragaria sp. plant, plant part, or plant cell of embodiment 113, wherein said plant, plant part, or plant cell comprises TFL1d1, TFL1d2, TFL1b1, and TFL1c edited alleles.
125. The cultivated Fragaria sp. plant, plant part, or plant cell of embodiment 113, wherein said plant, plant part, or plant cell comprises TFL1d1, TFL1d2, TFL1b1, and TFL1e edited alleles.
126. The cultivated Fragaria sp. plant, plant part, or plant cell of embodiment 113, wherein said plant, plant part, or plant cell comprises TFL1d1, TFL1d2, TFL1b2, and TFL1c edited alleles.
127. The cultivated Fragaria sp. plant, plant part, or plant cell of embodiment 113, wherein said plant, plant part, or plant cell comprises TFL1d1, TFL1d2, TFL1b2, and TFL1e edited alleles.
128. The cultivated Fragaria sp. plant, plant part, or plant cell of embodiment 113, wherein said plant, plant part, or plant cell comprises TFL1d1, TFL1d2, TFL1c, and TFL1e edited alleles.
129. The cultivated Fragaria sp. plant, plant part, or plant cell of embodiment 113, wherein said plant, plant part, or plant cell comprises TFL1d1, TFL1d2, TFL1a, TFL1b1, and TFL1b2 edited alleles.
130. The cultivated Fragaria sp. plant, plant part, or plant cell of embodiment 113, wherein said plant, plant part, or plant cell comprises TFL1d1, TFL1d2, TFL1a, TFL1b1, and TFL1c edited alleles.
131. The cultivated Fragaria sp. plant, plant part, or plant cell of embodiment 113, wherein said plant, plant part, or plant cell comprises TFL1d1, TFL1d2, TFL1a, TFL1b1, and TFL1e edited alleles.
132. The cultivated Fragaria sp. plant, plant part, or plant cell of embodiment 113, wherein said plant, plant part, or plant cell comprises TFL1d1, TFL1d2, TFL1a, TFL1b2, and TFL1c edited alleles.
133. The cultivated Fragaria sp. plant, plant part, or plant cell of embodiment 113, wherein said plant, plant part, or plant cell comprises TFL1d1, TFL1d2, TFL1a, TFL1b2, and TFL1e edited alleles.
134. The cultivated Fragaria sp. plant, plant part, or plant cell of embodiment 113, wherein said plant, plant part, or plant cell comprises TFL1d1, TFL1d2, TFL1a, TFL1c, and TFL1e edited alleles.
135. The cultivated Fragaria sp. plant, plant part, or plant cell of embodiment 113, wherein said plant, plant part, or plant cell comprises TFL1d1, TFL1d2, TFL1b1, TFL1b2, and TFL1c edited alleles.
136. The cultivated Fragaria sp. plant, plant part, or plant cell of embodiment 113, wherein said plant, plant part, or plant cell comprises TFL1d1, TFL1d2, TFL1b1, TFL1b2, and TFL1e edited alleles.
137. The cultivated Fragaria sp. plant, plant part, or plant cell of embodiment 113, wherein said plant, plant part, or plant cell comprises TFL1d1, TFL1d2, TFL1b1, TFL1c, and TFL1e edited alleles.
138. The cultivated Fragaria sp. plant, plant part, or plant cell of embodiment 113, wherein said plant, plant part, or plant cell comprises TFL1d1, TFL1d2, TFL1b2, TFL1c, and TFL1e edited alleles.
139. The cultivated Fragaria sp. plant, plant part, or plant cell of embodiment 113, wherein said plant, plant part, or plant cell comprises TFL1d1, TFL1d2, TFL1a, TFL1b1, TFL1b2, and TFL1c edited alleles.
140. The cultivated Fragaria sp. plant, plant part, or plant cell of embodiment 113, wherein said plant, plant part, or plant cell comprises TFL1d1, TFL1d2, TFL1a, TFL1b1, TFL1b2, and TFL1e edited alleles.
141. The cultivated Fragaria sp. plant, plant part, or plant cell of embodiment 113, wherein said plant, plant part, or plant cell comprises TFL1d1, TFL1d2, TFL1a, TFL1b1, TFL1c, and TFL1e edited alleles.
142. The cultivated Fragaria sp. plant, plant part, or plant cell of embodiment 113, wherein said plant, plant part, or plant cell comprises TFL1d1, TFL1d2, TFL1a, TFL1b2, TFL1c, and TFL1e edited alleles.
143. The cultivated Fragaria sp. plant, plant part, or plant cell of embodiment 113, wherein said plant, plant part, or plant cell comprises TFL1d1, TFL1d2, TFL1b1, TFL1b2, TFL1c, and TFL1e edited alleles.
144. The cultivated Fragaria sp. plant, plant part, or plant cell of embodiment 113, wherein said plant, plant part, or plant cell comprises TFL1d1, TFL1d2, TFL1a, TFL1b1, TFL1b2, TFL1c, and TFL1e edited alleles.
145. The cultivated Fragaria sp. plant, plant part, or plant cell of any one of embodiments 53-144, wherein the plant, plant part, or plant cell is a June-bearing variety, an early season June-bearing variety, an early midseason June-bearing variety, a midseason June-bearing variety, a late midseason June-bearing variety, a late season June-bearing variety, a short-day variety, a seasonal flowering variety, a long-day variety, a day-neutral variety, a perpetual flowering variety, a recurrent variety, a remontant variety, a long-day variety, or an everbearing variety.
146. The cultivated Fragaria sp. plant of any one of embodiments 53-145, wherein the plant flowers at least one week earlier than a cultivated Fragaria sp. plant of the same variety having wildtype TFL1d1 and TFL1d2 alleles when grown under the same environmental conditions.
147. The cultivated Fragaria sp. plant of any one of embodiments 53-145, wherein the plant flowers at least two weeks earlier than a cultivated Fragaria sp. plant of the same variety having wildtype TFL1d1 and TFL1d2 alleles when grown under the same environmental conditions.
148. The cultivated Fragaria sp. plant of any one of embodiments 53-145, wherein the plant flowers at least four weeks earlier than a cultivated Fragaria sp. plant of the same variety having wildtype TFL1d1 and TFL1d2 alleles when grown under the same environmental conditions.
149. The cultivated Fragaria sp. plant of any one of embodiments 53-145, wherein the plant flowers at least six weeks earlier than a cultivated Fragaria sp. plant of the same variety having wildtype TFL1d1 and TFL1d2 alleles when grown under the same environmental conditions.
150. The cultivated Fragaria sp. plant of any one of embodiments 53-145, wherein the plant flowers between six and 12 weeks earlier than a cultivated Fragaria sp. plant of the same variety having wildtype TFL1d1 and TFL1d2 alleles when grown under the same environmental conditions.
151. The cultivated Fragaria sp. plant of any one of embodiments 53-145, wherein the plant flowers between 12 and 20 weeks earlier than a cultivated Fragaria sp. plant of the same variety having wildtype TFL1d1 and TFL1d2 alleles when grown under the same environmental conditions.
152. The cultivated Fragaria sp. plant of any one of embodiments 53-145, wherein the plant flowers between 15 and 19 weeks earlier than a cultivated Fragaria sp. plant of the same variety having wildtype TFL1d1 and TFL1d2 alleles when grown under the same environmental conditions.
153. The cultivated Fragaria sp. plant of any one of embodiments 53-145, wherein the plant has increased yield compared to a cultivated Fragaria sp. plant of the same variety having wildtype TFL1d1 and TFL1d2 alleles when grown under the same environmental conditions.
154. The cultivated Fragaria sp. plant of embodiment 153, wherein the plant has between 1% and 10% increase in yield compared to a cultivated Fragaria sp. plant of the same variety having wildtype TFL1d1 and TFL1d2 alleles when grown under the same environmental conditions.
155. The cultivated Fragaria sp. plant of embodiment 153, wherein the plant has between 10% and 25% increase in yield compared to a cultivated Fragaria sp. plant of the same variety having wildtype TFL1d1 and TFL1d2 alleles when grown under the same environmental conditions.
156. The cultivated Fragaria sp. plant of embodiment 153, wherein the plant has between 25% and 50% increase in yield compared to a cultivated Fragaria sp. plant of the same variety having wildtype TFL1d1 and TFL1d2 alleles when grown under the same environmental conditions.
157. The cultivated Fragaria sp. plant of embodiment 153, wherein the plant has between 50% and 100% increase in yield compared to a cultivated Fragaria sp. plant of the same variety having wildtype TFL1d1 and TFL1d2 alleles when grown under the same environmental conditions.
158. The cultivated Fragaria sp. plant of embodiment 153, wherein the plant has between 100% and 500% increase in yield compared to a cultivated Fragaria sp. plant of the same variety having wildtype TFL1d1 and TFL1d2 alleles when grown under the same environmental conditions.
159. A method for producing a cultivated Rosaceae plant having increased yield, the method comprising:
   targeting one or more TFL1 alleles in a Rosaceae plant, plant part, or plant cell to reduce or knockout TFL1 function, wherein at least one of the targeted TFL1 alleles shares 80% or more sequence identity with SEQ ID NO: 62; and
   producing a cultivated Rosaceae plant therefrom, wherein the plant has increased yield compared to another cultivated Rosaceae plant of the same variety having wild-type TFL1 alleles and grown under the same conditions.
160. A method for producing a cultivated Rosaceae plant having an early flowering trait, the method comprising:
   targeting one or more TFL1 alleles in a Rosaceae plant, plant part, or plant cell to reduce or knockout TFL1 function, wherein at least one of the targeted TFL1 alleles shares 80% or more sequence identity with SEQ ID NO: 62; and
   producing a cultivated Rosaceae plant therefrom, wherein the plant flowers earlier compared to another cultivated Rosaceae plant of the same variety having wild-type TFL1 alleles and grown under the same conditions.
161. The method of embodiment 159 or 160, wherein the targeted TFL1 allele shares 85% or more sequence identity with SEQ ID NO: 62.
162. The method of embodiment 159 or 160, wherein the targeted TFL1 allele shares 90% or more sequence identity with SEQ ID NO: 62.
163. The method of embodiment 159 or 160, wherein the targeted TFL1 allele shares 95% or more sequence identity with SEQ ID NO: 62.
164. The method of embodiment 159 or 160, wherein the targeted TFL1 allele shares 96% or more sequence identity with SEQ ID NO: 62.
165. The method of embodiment 159 or 160, wherein the targeted TFL1 allele shares 97% or more sequence identity with SEQ ID NO: 62.
166. The method of embodiment 159 or 160, wherein the targeted TFL1 allele shares 98% or more sequence identity with SEQ ID NO: 62.
167. The method of embodiment 159 or 160, wherein the targeted TFL1 allele shares 99% or more sequence identity with SEQ ID NO: 62.
168. The method of any one of embodiments 159-167, wherein the targeting one or more TFL1 alleles results in: reduced gene expression level, reduced gene copy number, reduced gene amplification, reduced RNA activity level, reduced mRNA abundance, reduced mRNA synthesis rate, reduced mRNA stability, reduced protein activity level, reduced protein synthesis, reduced protein abundance, reduced protein stability, reduced substrate binding, reduced interaction with a 14-3-3 protein, or a combination thereof.
169. The method of any one of embodiments 159-168, wherein the targeting is RNA interference (RNAi), genome editing, or mutation of the endogenous TFL1 gene.
170. The method of embodiment 169, wherein the RNA interference is induced by expression in a cell of the cultivated Rosaceae plant an RNAi cassette targeting the endogenous TFL1 gene, or by topical application of RNAi triggers targeting the endogenous TFL1 gene.
171. The method of embodiment 169, wherein the genome editing is by expression in a cell of the cultivated Rosaceae plant of a zinc-finger nuclease, a TALE-mediated nuclease, or an RNA-guided nuclease.
172. The method of embodiment 168, wherein mutation of the endogenous TFL1 gene is by chemical mutagenesis, radiation mutagenesis, transposon mutagenesis, insertional mutagenesis, signature tagged mutagenesis, site-directed mutagenesis, and/or natural mutagenesis.
173. The method of any one of embodiments 159-172, wherein the method is plasmid-free.
174. The method of any one of embodiments 159-173, wherein the targeting comprising editing a region in exon 2 corresponding to between V67 and W87 of SEQ ID NO: 62.
175. The method of any one of embodiments 159-174, wherein all copies of a TFL1d1 allele or TFL1d1 homolog are targeted.
176. The method of any one of embodiments 159-175, wherein expression of all TFL1d1 alleles is reduced or knocked out.
177. The method of any one of embodiments 159-175, wherein each TFL1d1 allele has one or more edits that disrupt TFL1d1 protein interaction with a 14-3-3 protein.
178. The method of any one of embodiments 159-177, wherein all copies of a TFL1d2 allele or TFL1d2 homolog are targeted.
179. The method of any one of embodiments 159-178, wherein expression of all TFL1d2 alleles is reduced or knocked out.
180. The method of any one of embodiments 159-178, wherein each TFL1d2 allele has one or more edits that disrupt TFL1d2 protein interaction with a 14-3-3 protein.
181. The method of any one of embodiments 159-180, wherein the cultivated Rosaceae plant is a species of Fragaria.
182. The method of embodiment 181, wherein the plant is selected from F. iinumae, F. nipponica, F. pentaphylla, F. vesca, F. viridis, F. moupinensis, F. orientalis, F. moschata, F. chiloensis, F. iturupensis, F. virginiana, F. cascadensis, F. x ananassa, and hybrids thereof.
183. The method of embodiment 182, wherein the plant is selected from F. x ananassa, F. x bringhurstii, and F. x vescana.
184. The method of any one of embodiments 159-180, wherein the cultivated Rosaceae plant is a species of Rubus.
185. The method of embodiment 184, wherein the plant is selected from R. idaeus, R. allegheniensis, R. occidentalis, R. argutus, R. ursinus, R. laciniatus, R. ulmifolius, R. leucodermis, R. strigosus, R. ellipticus, R. subsp. rubus, and hybrids thereof.
186. The method of any one of embodiments 159-180, wherein the cultivated Rosaceae plant is a species of Vaccinium.
187. The method of embodiment 186, wherein the plant is selected from V. corymbosum, V. darrowii, V. angustifolium, V. ashei, and hybrids thereof.
188. The method of any one of embodiments 159-180, wherein the cultivated Rosaceae plant is a species of Malus.
189. The method of embodiment 188, wherein the plant is selected from M. domestica, and hybrids thereof.

While embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

1.-189. (canceled)

190. A cultivated Fragaria sp. plant, plant part, or plant cell having an early flowering trait, wherein said early flowering trait is caused by genetically engineered Terminal Flowering d1 and d2 alleles (TFL1d1 and TFL1d2), and wherein each TFL1d allele has one or more edits that reduce or knockout protein function.

191. The cultivated Fragaria sp. plant, plant part, or plant cell of claim 190, wherein said one or more edits that reduce or knockout protein function are located in exon 2.

192. A cultivated Fragaria sp. plant, plant part, or plant cell of claim 190, wherein each TFL1d allele has one or more edits that disrupt TFL protein interaction with a 14-3-3 protein.

193. The cultivated Fragaria sp. plant, plant part, or plant cell of claim 190, wherein the one or more edits comprise an insertion and/or a deletion in exon 2.

194. The cultivated Fragaria sp. plant, plant part, or plant cell of claim 190, wherein the one or more edits result in a frameshift mutation.

195. The cultivated Fragaria sp. plant, plant part, or plant cell of claim 190, wherein the one or more edits result in an early stop codon.

196. The cultivated Fragaria sp. plant, plant part, or plant cell of claim 190, wherein the one or more edits comprise a complete deletion of exon 2.

197. The cultivated Fragaria sp. plant, plant part, or plant cell of claim 190, wherein the engineered TFL1d alleles comprise at least one sequence selected from SEQ ID NOs: 1-20, SEQ ID NOs: 22-41, or sequences at least 75% identical thereto.

198. The cultivated Fragaria sp. plant, plant part, or plant cell of claim 197, wherein the engineered TFL1d alleles comprise SEQ ID NOs: 13-14, or sequences at least 75% identical thereto.

199. The cultivated Fragaria sp. plant, plant part, or plant cell of claim 190, wherein the engineered TFL1d alleles encode a protein sequence selected from SEQ ID NOs: 42-61, SEQ ID NOs: 63-82, or sequences at least 75% identical thereto.

200. The cultivated Fragaria sp. plant, plant part, or plant cell of claim 190, wherein the engineered TFL1d alleles encode SEQ ID NOs: 54-55, or sequences at least 75% identical thereto.

201. The cultivated Fragaria sp. plant, plant part, or plant cell of claim 190, wherein the plant, plant part, or plant cell further comprises one or more edits in a TFL1a, TFL1b1, TFL1b2, TFL1c, and/or TFL1e allele that reduce protein function.

202. The cultivated Fragaria sp. plant, plant part, or plant cell of claim 190, wherein said plant, plant part, or plant cell comprises TFL1d1, TFL1d2, TFL1a, TFL1b1, TFL1b2, TFL1c, and TFL1e edited alleles.

203. The cultivated Fragaria sp. plant of any one claim 190, wherein the plant flowers at least one week earlier than a cultivated Fragaria sp. plant of the same variety having wildtype TFL1d1 and TFL1d2 alleles when grown under the same environmental conditions.

204. The cultivated Fragaria sp. plant of claim 190, wherein the plant has increased yield compared to a cultivated Fragaria sp. plant of the same variety having wildtype TFL1d1 and TFL1d2 alleles when grown under the same environmental conditions.

205. The cultivated Fragaria sp. plant, plant part, or plant cell of claim 190, wherein the plant, plant part, or plant cell is selected from F. x ananassa, F. x bringhurstii, and F. x vescana.

206. A method for producing a cultivated Fragaria sp. plant having an early flowering trait, the method comprising:
targeting one or more TFL1 alleles in the Fragaria sp. plant, plant part, or plant cell to reduce or knockout TFL1 function, wherein at least one of the targeted TFL1 alleles shares 80% or more sequence identity with SEQ ID NO: 62; and
producing a cultivated Fragaria sp. plant therefrom, wherein the plant flowers earlier compared to another cultivated Fragaria sp. plant of the same variety having wild-type TFL1 alleles and grown under the same conditions.

207. The method of claim 206, wherein the targeting is RNA interference (RNAi), genome editing, or mutation of an endogenous TFL1 gene.

208. The method of claim 206, wherein the method is plasmid-free.

209. The method of claim 206, wherein the targeting comprising editing a region in exon 2 corresponding to between V67 and W87 of SEQ ID NO: 62.


SEQUENCE LISTING (117 sequences)

SEQ ID NO: 1 - DNA, 1090 bp, Fragaria sp.
  source 1..1090: mol_type = other DNA
atggcaagaatgtcggaacctctagctgttggaagagtcataggagatgttcttgattccttcaccccaactacaaaaatgattgtcacttacaacaccaagctcgtctgcaatggacatgagctcttcccttctgcagtcaccgccaaacctagagttgagattcaaggaggcgacatgagatcattcttcactctggtatacatcattttctttctggcctccttttccgccgtttgttttttttatcagatctaccagatcgctaactttagttattttcacaggtaatgacagaccctggccctagtgatccttatttgaaagagcacctgcactggtatatcttacagttaatactgtctatatattatcaaacctagaaacgaagaaacaaagacgataactattcagataattattaataacttcaatgttcaaactagatatatatgttgtcatatactagaaatttgcagtattgaccgaacacattgtccaggttaatttatagaaaagattagttatattaattattgttaatttattctttctcctttacatggtcattcacgtatatattcagaattgtaatcagaaaatctttggatctggaaataaaagctagtaaaaattacatattatgtcacgaactcttagttataatggtggagaatatacatgccactaagcttggatctaacagttttgccttgaaattgaactatacaggattgtgacagacattcctggcaccacagatgctacatttggtaggttaattcacactagttcatgagctaccagtacgttatacagctagtgatgatcagcactgatattgtacgtgtatatactacattaattaacaggaagagaagtggtgagctacgagatgccaaggccaaacataggcatccacaggtttgtgtttgttctcttcatgcaaaaacgaaggcagtcggtgaacccgccttcctcacgggatcacttcaacacccgaaccttcgcagccgaaaacgaccttggtgttcctgttgctgccgtttacttcaatgcacagagagaaacggcagcaagaagacgctag

SEQ ID NO: 2 - DNA, 1100 bp, Fragaria sp.
  source 1..1100: mol_type = other DNA
atggcaagaatgtcggaacctctagctgttggaagagtcataggagatgttcttgattccttcaccccaactacaaaaatgattgtcacttacaacaccaagctcgtctgcaatggacatgagctcttcccttctgcagtcaccgccaaacctagagttgagattcaaggaggcgacatgagatcattcttcactctggtatacatcattttctttctggcctccttttccgccgtttgttttttttatcagatctaccagatcgctaactttagttattttcacaggtaatgacagacccagattgttcctggccctagtgatccttatttgaaagagcacctgcactggtatatcttacagttaatactgtctatatattatcaaacctagaaacgaagaaacaaagacgataactattcagataattattaataacttcaatgttcaaactagatatatatgttgtcatatactagaaatttgcagtattgaccgaacacattgtccaggttaatttatagaaaagattagttatattaattattgttaatttattctttctcctttacatggtcattcacgtatatattcagaattgtaatcagaaaatctttggatctggaaataaaagctagtaaaaattacatattatgtcacgaactcttagttataatggtggagaatatacatgccactaagcttggatctaacagttttgccttgaaattgaactatacaggattgtgacagacattcctggcaccacagatgctacatttggtaggttaattcacactagttcatgagctaccagtacgttatacagctagtgatgatcagcactgatattgtacgtgtatatactacattaattaacaggaagagaagtggtgagctacgagatgccaaggccaaacataggcatccacaggtttgtgtttgttctcttcatgcaaaaacgaaggcagtcggtgaacccgccttcctcacgggatcacttcaacacccgaaccttcgcagccgaaaacgaccttggtgttcctgttgctgccgtttacttcaatgcacagagagaaacggcagcaagaagacgctag

SEQ ID NO: 3 - DNA, 1096 bp, Fragaria sp.
  source 1..1096: mol_type = other DNA
atggcaagaatgtcggaacctctagctgttggaagagtcataggagatgttcttgattccttcaccccaactacaaaaatgattgtcacttacaacaccaagctcgtctgcaatggacatgagctcttcccttctgcagtcaccgccaaacctagagttgagattcaaggaggcgacatgagatcattcttcactctggtatacatcattttctttctggcctccttttccgccgtttgttttttttatcagatctaccagatcgctaactttagttattttcacaggtaatgacagacccagatcctggccctagtgatccttatttgaaagagcacctgcactggtatatcttacagttaatactgtctatatattatcaaacctagaaacgaagaaacaaagacgataactattcagataattattaataacttcaatgttcaaactagatatatatgttgtcatatactagaaatttgcagtattgaccgaacacattgtccaggttaatttatagaaaagattagttatattaattattgttaatttattctttctcctttacatggtcattcacgtatatattcagaattgtaatcagaaaatctttggatctggaaataaaagctagtaaaaattacatattatgtcacgaactcttagttataatggtggagaatatacatgccactaagcttggatctaacagttttgccttgaaattgaactatacaggattgtgacagacattcctggcaccacagatgctacatttggtaggttaattcacactagttcatgagctaccagtacgttatacagctagtgatgatcagcactgatattgtacgtgtatatactacattaattaacaggaagagaagtggtgagctacgagatgccaaggccaaacataggcatccacaggtttgtgtttgttctcttcatgcaaaaacgaaggcagtcggtgaacccgccttcctcacgggatcacttcaacacccgaaccttcgcagccgaaaacgaccttggtgttcctgttgctgccgtttacttcaatgcacagagagaaacggcagcaagaagacgctag

SEQ ID NO: 4 - DNA, 1100 bp, Fragaria sp.; identical to SEQ ID NO: 2
  source 1..1100: mol_type = other DNA

SEQ ID NO: 5 - DNA, 1056 bp, Fragaria sp.
  source 1..1056: mol_type = other DNA
atggcaagaatgtcggaacctctagctgttggaagagtcataggagatgttcttgattccttcaccccaactacaaaaatgattgtcacttacaacaccaagctcgtctgcaatggacatgagctcttcccttctgcagtcaccgccaaacctagagttgagattcaaggaggcgacatgagatcattcttcactctggtatacatcattttctttctggcctccttttccgccgtttgttttttttatcagatctaccagatcgctaactttagttattttcacaggtaaagagcacctgcactggtatatcttacagttaatactgtctatatattatcaaacctagaaacgaagaaacaaagacgataactattcagataattattaataacttcaatgttcaaactagatatatatgttgtcatatactagaaatttgcagtattgaccgaacacattgtccaggttaatttatagaaaagattagttatattaattattgttaatttattctttctcctttacatggtcattcacgtatatattcagaattgtaatcagaaaatctttggatctggaaataaaagctagtaaaaattacatattatgtcacgaactcttagttataatggtggagaatatacatgccactaagcttggatctaacagttttgccttgaaattgaactatacaggattgtgacagacattcctggcaccacagatgctacatttggtaggttaattcacactagttcatgagctaccagtacgttatacagctagtgatgatcagcactgatattgtacgtgtatatactacattaattaacaggaagagaagtggtgagctacgagatgccaaggccaaacataggcatccacaggtttgtgtttgttctcttcatgcaaaaacgaaggcagtcggtgaacccgccttcctcacgggatcacttcaacacccgaaccttcgcagccgaaaacgaccttggtgttcctgttgctgccgtttacttcaatgcacagagagaaacggcagcaagaagacgctag

SEQ ID NO: 6 - DNA, 1096 bp, Fragaria sp.
  source 1..1096: mol_type = other DNA
atggcaagaatgtcggaacctctagctgttggaagagtcataggagatgttcttgattccttcaccccaactacaaaaatgattgtcacttacaacaccaagctcgtctgcaatggacatgagctcttcccttctgcagtcaccgccaaacctagagttgagattcaaggaggcgacatgagatcattcttcactctggtatacatcattttctttctggcctccttttccgccgtttgttttttttatcagatctaccagatcgctaactttagttattttcacaggtaatgacagacccagagcctggccctagtgatccttatttgaaagagcacctgcactggtatatcttacagttaatactgtctatatattatcaaacctagaaacgaagaaacaaagacgataactattcagataattattaataacttcaatgttcaaactagatatatatgttgtcatatactagaaatttgcagtattgaccgaacacattgtccaggttaatttatagaaaagattagttatattaattattgttaatttattctttctcctttacatggtcattcacgtatatattcagaattgtaatcagaaaatctttggatctggaaataaaagctagtaaaaattacatattatgtcacgaactcttagttataatggtggagaatatacatgccactaagcttggatctaacagttttgccttgaaattgaactatacaggattgtgacagacattcctggcaccacagatgctacatttggtaggttaattcacactagttcatgagctaccagtacgttatacagctagtgatgatcagcactgatattgtacgtgtatatactacattaattaacaggaagagaagtggtgagctacgagatgccaaggccaaacataggcatccacaggtttgtgtttgttctcttcatgcaaaaacgaaggcagtcggtgaacccgccttcctcacgggatcacttcaacacccgaaccttcgcagccgaaaacgaccttggtgttcctgttgctgccgtttacttcaatgcacagagagaaacggcagcaagaagacgctag

SEQ ID NO: 7 - DNA, 1097 bp, Fragaria sp.
  source 1..1097: mol_type = other DNA
atggcaagaatgtcggaacctctagctgttggaagagtcataggagatgttcttgattccttcaccccaactacaaaaatgattgtcacttacaacaccaagctcgtctgcaatggacatgagctcttcccttctgcagtcaccgccaaacctagagttgagattcaaggaggcgacatgagatcattcttcactctggtatacatcattttctttctggcctccttttccgccgtttgtttttttttatcagatctaccagatcgctaactttagttattttcacaggtaatgacagacccagttcctggccctagtgatccttatttgaaagagcacctgcactggtatatcttacagttaatactgtctatatattatcaaacctagaaacgaagaaacaaagacgataactattcagataattattaataacttcaatgttcaaactagatatatatgttgtcatatactagaaatttgcagtattgaccgaacacattgtccaggttaatttatagaaaagattagttatattaattattgttaatttattctttctcctttacatggtcattcacgtatatattcagaattgtaatcagaaaatctttggatctggaaataaaagctagtaaaaattacatattatgtcacgaactcttagttataatggtggagaatatacatgccactaagcttggatctaacagttttgccttgaaattgaactatacaggattgtgacagacattcctggcaccacagatgctacatttggtaggttaattcacactagttcatgagctaccagtacgttatacagctagtgatgatcagcactgatattgtacgtgtatatactacattaattaacaggaagagaagtggtgagctacgagatgccaaggccaaacataggcatccacaggtttgtgtttgttctcttcatgcaaaaacgaaggcagtcggtgaacccgccttcctcacgggatcacttcaacacccgaaccttcgcagccgaaaacgaccttggtgttcctgttgctgccgtttacttcaatgcacagagagaaacggcagcaagaagacgctag

SEQ ID NO: 8 - DNA, 1096 bp, Fragaria sp.
  source 1..1096: mol_type = other DNA
atggcaagaatgtcggaacctctagctgttggaagagtcataggagatgttcttgattccttcaccccaactacaaaaatgattgtcacttacaacaccaagctcgtctgcaatggacatgagctcttcccttctgcagtcaccgccaaacctagagttgagattcaaggaggcgacatgagatcattcttcactctggtatacatcattttctttctggcctccttttccgccgtttgtttttttttatcagatctaccagatcgctaactttagttattttcacaggtaatgacagacccagacctggccctagtgatccttatttgaaagagcacctgcactggtatatcttacagttaatactgtctatatattatcaaacctagaaacgaagaaacaaagacgataactattcagataattattaataacttcaatgttcaaactagatatatatgttgtcatatactagaaatttgcagtattgaccgaacacattgtccaggttaatttatagaaaagattagttatattaattattgttaatttattctttctcctttacatggtcattcacgtatatattcagaattgtaatcagaaaatctttggatctggaaataaaagctagtaaaaattacatattatgtcacgaactcttagttataatggtggagaatatacatgccactaagcttggatctaacagttttgccttgaaattgaactatacaggattgtgacagacattcctggcaccacagatgctacatttggtaggttaattcacactagttcatgagctaccagtacgttatacagctagtgatgatcagcactgatattgtacgtgtatatactacattaattaacaggaagagaagtggtgagctacgagatgccaaggccaaacataggcatccacaggtttgtgtttgttctcttcatgcaaaaacgaaggcagtcggtgaacccgccttcctcacgggatcacttcaacacccgaaccttcgcagccgaaaacgaccttggtgttcctgttgctgccgtttacttcaatgcacagagagaaacggcagcaagaagacgctag

SEQ ID NO: 9 - DNA, 1097 bp, Fragaria sp.; identical to SEQ ID NO: 7
  source 1..1097: mol_type = other DNA

SEQ ID NO: 10 - DNA, 1101 bp, Fragaria sp.
  source 1..1101: mol_type = other DNA
atgatggcaagaatgtcggaacctctagctgttggaagagtcataggagatgttcttgattccttcaccccaactacaaaaatgattgtcacttacaacaccaagctcgtctgcaatggacatgagctcttcccttctgcagtcaccgccaaacctagagttgagattcaaggaggcgacatgagatcattcttcactctggtatacatcattttctttctggcctccttttccgccgtttgtttttttttatcagatctaccagatcgctaactttagttattttcacaggtaatgacagacccaggttcctggccctagtgatccttatttgaaagagcacctgcactggtatatcttacagttaatactgtctatatattatcaaacctagaaacgaagaaacaaagacgataactattcagataattattaataacttcaatgttcaaactagatatatatgttgtcatatactagaaatttgcagtattgaccgaacacattgtccaggttaatttatagaaaagattagttatattaattattgttaatttattctttctcctttacatggtcattcacgtatatattcagaattgtaatcagaaaatctttggatctggaaataaaagctagtaaaaattacatattatgtcacgaactcttagttataatggtggagaatatacatgccactaagcttggatctaacagttttgccttgaaattgaactatacaggattgtgacagacattcctggcaccacagatgctacatttggtaggttaattcacactagttcatgagctaccagtacgttatacagctagtgatgatcagcactgatattgtacgtgtatatactacattaattaacaggaagagaagtggtgagctacgagatgccaaggccaaacataggcatccacaggtttgtgtttgttctcttcatgcaaaaacgaaggcagtcggtgaacccgccttcctcacgggatcacttcaacacccgaaccttcgcagccgaaaacgaccttggtgttcctgttgctgccgtttacttcaatgcacagagagaaacggcagcaagaagacgctag

SEQ ID NO: 11 - DNA, 1097 bp, Fragaria sp.; identical to SEQ ID NO: 7
  source 1..1097: mol_type = other DNA

SEQ ID NO: 12 - DNA, 1097 bp, Fragaria sp.
  source 1..1097: mol_type = other DNA
atggcaagaatgtcggaacctctagctgttggaagagtcataggagatgttcttgattccttcaccccaactacaaaaatgattgtcacttacaacaccaagctcgtctgcaatggacatgagctcttcccttctgcagtcaccgccaaacctagagttgagattcaaggaggcgacatgagatcattcttcactctggtatacatcattttctttctggcctccttttccgccgtttgttttttttatcagatctaccagatcgctaactttagttattttcacaggtaatgacagacccagattcctggccctagtgatccttatttgaaagagcacctgcactggtatatcttacagttaatactgtctatatattatcaaacctagaaacgaagaaacaaagacgataactattcagataattattaataacttcaatgttcaaactagatatatatgttgtcatatactagaaatttgcagtattgaccgaacacattgtccaggttaatttatagaaaagattagttatattaattattgttaatttattctttctcctttacatggtcattcacgtatatattcagaattgtaatcagaaaatctttggatctggaaataaaagctagtaaaaattacatattatgtcacgaactcttagttataatggtggagaatatacatgccactaagcttggatctaacagttttgccttgaaattgaactatacaggattgtgacagacattcctggcaccacagatgctacatttggtaggttaattcacactagttcatgagctaccagtacgttatacagctagtgatgatcagcactgatattgtacgtgtatatactacattaattaacaggaagagaagtggtgagctacgagatgccaaggccaaacataggcatccacaggtttgtgtttgttctcttcatgcaaaaacgaaggcagtcggtgaacccgccttcctcacgggatcacttcaacacccgaaccttcgcagccgaaaacgaccttggtgttcctgttgctgccgtttacttcaatgcacagagagaaacggcagcaagaagacgctag

SEQ ID NO: 13 - DNA, 1096 bp, Fragaria sp.; identical to SEQ ID NO: 3
  source 1..1096: mol_type = other DNA

SEQ ID NO: 14 - DNA, 1098 bp, Fragaria sp.
  source 1..1098: mol_type = other DNA
atggcaagaatgtcggaacctctagctgttggaagagtcataggagatgttcttgattccttcaccccaactacaaaaatgattgtcacttacaacaccaagctcgtctgcaatggacatgagctcttcccttctgcagtcaccgccaaacctagagttgagattcaaggaggcgacatgagatcattcttcactctggtatacatcattttctttctggcctccttttccgccgtttgttttttttatcagatctaccagatcgctaactttagttattttcacaggtaatgacagacccagagttcctggccctagtgatccttatttgaaagagcacctgcactggtatatcttacagttaatactgtctatatattatcaaacctagaaacgaagaaacaaagacgataactattcagataattattaataacttcaatgttcaaactagatatatatgttgtcatatactagaaatttgcagtattgaccgaacacattgtccaggttaatttatagaaaagattagttatattaattattgttaatttattctttctcctttacatggtcattcacgtatatattcagaattgtaatcagaaaatctttggatctggaaataaaagctagtaaaaattacatattatgtcacgaactcttagttataatggtggagaatatacatgccactaagcttggatctaacagttttgccttgaaattgaactatacaggattgtgacagacattcctggcaccacagatgctacatttggtaggttaattcacactagttcatgagctaccagtacgttatacagctagtgatgatcagcactgatattgtacgtgtatatactacattaattaacaggaagagaagtggtgagctacgagatgccaaggccaaacataggcatccacaggtttgtgtttgttctcttcatgcaaaaacgaaggcagtcggtgaacccgccttcctcacgggatcacttcaacacccgaaccttcgcagccgaaaacgaccttggtgttcctgttgctgccgtttacttcaatgcacagagagaaacggcagcaagaagacgctag

SEQ ID NO: 15 - DNA, 1091 bp, Fragaria sp.
  source 1..1091: mol_type = other DNA
atggcaagaatgtcggaacctctagctgttggaagagtcataggagatgttcttgattccttcaccccaactacaaaaatgattgtcacttacaacaccaagctcgtctgcaatggacatgagctcttcccttctgcagtcaccgccaaacctagagttgagattcaaggaggcgacatgagatcattcttcactctggtatacatcattttctttctggcctccttttccgccgtttgtttttttttatcagatctaccagatcgctaactttagttattttcacaggtaatgacagaccctggccctagtgatccttatttgaaagagcacctgcactggtatatcttacagttaatactgtctatatattatcaaacctagaaacgaagaaacaaagacgataactattcagataattattaataacttcaatgttcaaactagatatatatgttgtcatatactagaaatttgcagtattgaccgaacacattgtccaggttaatttatagaaaagattagttatattaattattgttaatttattctttctcctttacatggtcattcacgtatatattcagaattgtaatcagaaaatctttggatctggaaataaaagctagtaaaaattacatattatgtcacgaactcttagttataatggtggagaatatacatgccactaagcttggatctaacagttttgccttgaaattgaactatacaggattgtgacagacattcctggcaccacagatgctacatttggtaggttaattcacactagttcatgagctaccagtacgttatacagctagtgatgatcagcactgatattgtacgtgtatatactacattaattaacaggaagagaagtggtgagctacgagatgccaaggccaaacataggcatccacaggtttgtgtttgttctcttcatgcaaaaacgaaggcagtcggtgaacccgccttcctcacgggatcacttcaacacccgaaccttcgcagccgaaaacgaccttggtgttcctgttgctgccgtttacttcaatgcacagagagaaacggcagcaagaagacgctag

SEQ ID NO: 16 - DNA, 1096 bp, Fragaria sp.; identical to SEQ ID NO: 8
  source 1..1096: mol_type = other DNA

SEQ ID NO: 17 - DNA, 1097 bp, Fragaria sp.
  source 1..1097: mol_type = other DNA
atggcaagaatgtcggaacctctagctgttggaagagtcataggagatgttcttgattccttcaccccaactacaaaaatgattgtcacttacaacaccaagctcgtctgcaatggacatgagctcttcccttctgcagtcaccgccaaacctagagttgagattcaaggaggcgacatgagatcattcttcactctggtatacatcattttctttctggcctccttttccgccgtttgtttttttttatcagatctaccagatcgctaactttagttattttcacaggtaatgacagacccagatcctggccctagtgatccttatttgaaagagcacctgcactggtatatcttacagttaatactgtctatatattatcaaacctagaaacgaagaaacaaagacgataactattcagataattattaataacttcaatgttcaaactagatatatatgttgtcatatactagaaatttgcagtattgaccgaacacattgtccaggttaatttatagaaaagattagttatattaattattgttaatttattctttctcctttacatggtcattcacgtatatattcagaattgtaatcagaaaatctttggatctggaaataaaagctagtaaaaattacatattatgtcacgaactcttagttataatggtggagaatatacatgccactaagcttggatctaacagttttgccttgaaattgaactatacaggattgtgacagacattcctggcaccacagatgctacatttggtaggttaattcacactagttcatgagctaccagtacgttatacagctagtgatgatcagcactgatattgtacgtgtatatactacattaattaacaggaagagaagtggtgagctacgagatgccaaggccaaacataggcatccacaggtttgtgtttgttctcttcatgcaaaaacgaaggcagtcggtgaacccgccttcctcacgggatcacttcaacacccgaaccttcgcagccgaaaacgaccttggtgttcctgttgctgccgtttacttcaatgcacagagagaaacggcagcaagaagacgctag

SEQ ID NO: 18 - DNA, 1096 bp, Fragaria sp.
  source 1..1096: mol_type = other DNA
atggcaagaatgtcggaacctctagctgttggaagagtcataggagatgttcttgattccttcaccccaactacaaaaatgattgtcacttacaacaccaagctcgtctgcaatggacatgagctcttcccttctgcagtcaccgccaaacctagagttgagattcaaggaggcgacatgagatcattcttcactctggtatacatcattttctttctggcctccttttccgccgtttgtttttttttatcagatctaccagatcgctaactttagttattttcacaggtaatgacagacccagtcctggccctagtgatccttatttgaaagagcacctgcactggtatatcttacagttaatactgtctatatattatcaaacctagaaacgaagaaacaaagacgataactattcagataattattaataacttcaatgttcaaactagatatatatgttgtcatatactagaaatttgcagtattgaccgaacacattgtccaggttaatttatagaaaagattagttatattaattattgttaatttattctttctcctttacatggtcattcacgtatatattcagaattgtaatcagaaaatctttggatctggaaataaaagctagtaaaaattacatattatgtcacgaactcttagttataatggtggagaatatacatgccactaagcttggatctaacagttttgccttgaaattgaactatacaggattgtgacagacattcctggcaccacagatgctacatttggtaggttaattcacactagttcatgagctaccagtacgttatacagctagtgatgatcagcactgatattgtacgtgtatatactacattaattaacaggaagagaagtggtgagctacgagatgccaaggccaaacataggcatccacaggtttgtgtttgttctcttcatgcaaaaacgaaggcagtcggtgaacccgccttcctcacgggatcacttcaacacccgaaccttcgcagccgaaaacgaccttggtgttcctgttgctgccgtttacttcaatgcacagagagaaacggcagcaagaagacgctag

SEQ ID NO: 19 - DNA, 1088 bp, Fragaria sp.
  source 1..1088: mol_type = other DNA
atggcaagaatgtcggaacctctagctgttggaagagtcataggagatgttcttgattccttcaccccaactacaaaaatgattgtcacttacaacaccaagctcgtctgcaatggacatgagctcttcccttctgcagtcaccgccaaacctagagttgagattcaaggaggcgacatgagatcattcttcactctggtatacatcattttctttctggcctccttttccgccgtttgtttttttttatcagatctaccagatcgctaactttagttattttcacaggtaatgacacctggccctagtgatccttatttgaaagagcacctgcactggtatatcttacagttaatactgtctatatattatcaaacctagaaacgaagaaacaaagacgataactattcagataattattaataacttcaatgttcaaactagatatatatgttgtcatatactagaaatttgcagtattgaccgaacacattgtccaggttaatttatagaaaagattagttatattaattattgttaatttattctttctcctttacatggtcattcacgtatatattcagaattgtaatcagaaaatctttggatctggaaataaaagctagtaaaaattacatattatgtcacgaactcttagttataatggtggagaatatacatgccactaagcttggatctaacagttttgccttgaaattgaactatacaggattgtgacagacattcctggcaccacagatgctacatttggtaggttaattcacactagttcatgagctaccagtacgttatacagctagtgatgatcagcactgatattgtacgtgtatatactacattaattaacaggaagagaagtggtgagctacgagatgccaaggccaaacataggcatccacaggtttgtgtttgttctcttcatgcaaaaacgaaggcagtcggtgaacccgccttcctcacgggatcacttcaacacccgaaccttcgcagccgaaaacgaccttggtgttcctgttgctgccgtttacttcaatgcacagagagaaacggcagcaagaagacgctag

SEQ ID NO: 20 - DNA, 1097 bp, Fragaria sp.; identical to SEQ ID NO: 17
  source 1..1097: mol_type = other DNA

SEQ ID NO: 21 - DNA, 1099 bp, Fragaria sp.
  source 1..1099: mol_type = other DNA
atggcaagaatgtcggaacctctagctgttggaagagtcataggagatgttcttgattccttcaccccaactacaaaaatgattgtcacttacaacaccaagctcgtctgcaatggacatgagctcttcccttctgcagtcaccgccaaacctagagttgagattcaaggaggcgacatgagatcattcttcactctggtatacatcattttctttctggcctccttttccgccgtttgttttttttatcagatctaccagatcgctaactttagttattttcacaggtaatgacagacccagatgttcctggccctagtgatccttatttgaaagagcacctgcactggtatatcttacagttaatactgtctatatattatcaaacctagaaacgaagaaacaaagacgataactattcagataattattaataacttcaatgttcaaactagatatatatgttgtcatatactagaaatttgcagtattgaccgaacacattgtccaggttaatttatagaaaagattagttatattaattattgttaatttattctttctcctttacatggtcattcacgtatatattcagaattgtaatcagaaaatctttggatctggaaataaaagctagtaaaaattacatattatgtcacgaactcttagttataatggtggagaatatacatgccactaagcttggatctaacagttttgccttgaaattgaactatacaggattgtgacagacattcctggcaccacagatgctacatttggtaggttaattcacactagttcatgagctaccagtacgttatacagctagtgatgatcagcactgatattgtacgtgtatatactacattaattaacaggaagagaagtggtgagctacgagatgccaaggccaaacataggcatccacaggtttgtgtttgttctcttcatgcaaaaacgaaggcagtcggtgaacccgccttcctcacgggatcacttcaacacccgaaccttcgcagccgaaaacgaccttggtgttcctgttgctgccgtttacttcaatgcacagagagaaacggcagcaagaagacgctag

SEQ ID NO: 22 - DNA, 1099 bp, Fragaria sp.
  source 1..1099: mol_type = other DNA
atggcaagaatgtcggaacctctagctgttggaagagtcataggagatgttcttgattccttcaccccaactacaaaaatgattgtcacttacaacaccaagctcgtctgcaatggacatgagctcttcccttctgcagtcaccgccaaacctagagttgagattcaaggaggcgacatgagatcattcttcactctggtatacatcattttctttctggcctccttttccgccgtttgtttttttttatcagatctaccagatcgctaactttagttattttcacaggtaatgacagacccagagttcctggccctagtgatccttatttgaaagagcacctgcactggtatatcttacagttaatactgtctatatattatcaaacctagaaacgaagaaacaaagacgataactattcagataattattaataacttcaatgttcaaactagatatatatgttgtcatatactagaaatttgcagtattgaccgaacacattgtccaggttaatttatagaaaagattagttatattaattattgttaatttattctttctcctttacatggtcattcacgtatatattcagaattgtaatcagaaaatctttggatctggaaataaaagctagtaaaaattacatattatgtcacgaactcttagttataatggtggagaatatacatgccactaagcttggatctaacagttttgccttgaaattgaactatacaggattgtgacagacattcctggcaccacagatgctacatttggtaggttaattcacactagttcatgagctaccagtacgttatacagctagtgatgatcagcactgatattgtacgtgtatatactacattaattaacaggaagagaagtggtgagctacgagatgccaaggccaaacataggcatccacaggtttgtgtttgttctcttcatgcaaaaacgaaggcagtcggtgaacccgccttcctcacgggatcacttcaacacccgaaccttcgcagccgaaaacgaccttggtgttcctgttgctgccgtttacttcaatgcacagagagaaacggcagcaagaagacgctag

SEQ ID NO: 23 - DNA, 1098 bp, Fragaria sp.
  source 1..1098: mol_type = other DNA
atggcaagaatgtcggaacctctagctgttggaagagtcataggagatgttcttgattccttcaccccaactacaaaaatgattgtcacttacaacaccaagctcgtctgcaatggacatgagctcttcccttctgcagtcaccgccaaacctagagttgagattcaaggaggcgacatgagatcattcttcactctggtatacatcattttctttctggcctccttttccgccgtttgtttttttttatcagatctaccagatcgctaactttagttattttcacaggtaatgacagacccagacccctggccctagtgatccttatttgaaagagcacctgcactggtatatcttacagttaatactgtctatatattatcaaacctagaaacgaagaaacaaagacgataactattcagataattattaataacttcaatgttcaaactagatatatatgttgtcatatactagaaatttgcagtattgaccgaacacattgtccaggttaatttatagaaaagattagttatattaattattgttaatttattctttctcctttacatggtcattcacgtatatattcagaattgtaatcagaaaatctttggatctggaaataaaagctagtaaaaattacatattatgtcacgaactcttagttataatggtggagaatatacatgccactaagcttggatctaacagttttgccttgaaattgaactatacaggattgtgacagacattcctggcaccacagatgctacatttggtaggttaattcacactagttcatgagctaccagtacgttatacagctagtgatgatcagcactgatattgtacgtgtatatactacattaattaacaggaagagaagtggtgagctacgagatgccaaggccaaacataggcatccacaggtttgtgtttgttctcttcatgcaaaaacgaaggcagtcggtgaacccgccttcctcacgggatcacttcaacacccgaaccttcgcagccgaaaacgaccttggtgttcctgttgctgccgtttacttcaatgcacagagagaaacggcagcaagaagacgctag

SEQ ID NO: 24 - DNA, 1090 bp, Fragaria sp.
  source 1..1090: mol_type = other DNA
atggcaagaatgtcggaacctctagctgttggaagagtcataggagatgttcttgattccttcaccccaactacaaaaatgattgtcacttacaacaccaagctcgtctgcaatggacatgagctcttcccttctgcagtcaccgccaaacctagagttgagattcaaggaggcgacatgagatcattcttcactctggtatacatcattttctttctggcctccttttccgccgtttgtttttttttatcagatctaccagatcgctaactttagttattttcacaggtaatgacagacccagccctagtgatccttatttgaaagagcacctgcactggtatatcttacagttaatactgtctatatattatcaaacctagaaacgaagaaacaaagacgataactattcagataattattaataacttcaatgttcaaactagatatatatgttgtcatatactagaaatttgcagtattgaccgaacacattgtccaggttaatttatagaaaagattagttatattaattattgttaatttattctttctcctttacatggtcattcacgtatatattcagaattgtaatcagaaaatctttggatctggaaataaaagctagtaaaaattacatattatgtcacgaactcttagttataatggtggagaatatacatgccactaagcttggatctaacagttttgccttgaaattgaactatacaggattgtgacagacattcctggcaccacagatgctacatttggtaggttaattcacactagttcatgagctaccagtacgttatacagctagtgatgatcagcactgatattgtacgtgtatatactacattaattaacaggaagagaagtggtgagctacgagatgccaaggccaaacataggcatccacaggtttgtgtttgttctcttcatgcaaaaacgaaggcagtcggtgaacccgccttcctcacgggatcacttcaacacccgaaccttcgcagccgaaaacgaccttggtgttcctgttgctgccgtttacttcaatgcacagagagaaacggcagcaagaagacgctag

SEQ ID NO: 25 - DNA, 1095 bp, Fragaria sp.
  source 1..1095: mol_type = other DNA
atggcaagaatgtcggaacctctagctgttggaagagtcataggagatgttcttgattccttcaccccaactacaaaaatgattgtcacttacaacaccaagctcgtctgcaatggacatgagctcttcccttctgcagtcaccgccaaacctagagttgagattcaaggaggcgacatgagatcattcttcactctggtatacatcattttctttctggcctccttttccgccgtttgtttttttttatcagatctaccagatcgctaactttagttattttcacaggtaatgacagacccagcctggccctagtgatccttatttgaaagagcacctgcactggtatatcttacagttaatactgtctatatattatcaaacctagaaacgaagaaacaaagacgataactattcagataattattaataacttcaatgttcaaactagatatatatgttgtcatatactagaaatttgcagtattgaccgaacacattgtccaggttaatttatagaaaagattagttatattaattattgttaatttattctttctcctttacatggtcattcacgtatatattcagaattgtaatcagaaaatctttggatctggaaataaaagctagtaaaaattacatattatgtcacgaactcttagttataatggtggagaatatacatgccactaagcttggatctaacagttttgccttgaaattgaactatacaggattgtgacagacattcctggcaccacagatgctacatttggtaggttaattcacactagttcatgagctaccagtacgttatacagctagtgatgatcagcactgatattgtacgtgtatatactacattaattaacaggaagagaagtggtgagctacgagatgccaaggccaaacataggcatccacaggtttgtgtttgttctcttcatgcaaaaacgaaggcagtcggtgaacccgccttcctcacgggatcacttcaacacccgaaccttcgcagccgaaaacgaccttggtgttcctgttgctgccgtttacttcaatgcacagagagaaacggcagcaagaagacgctag

SEQ ID NO: 26 - DNA, 1098 bp, Fragaria sp.; identical to SEQ ID NO: 14
  source 1..1098: mol_type = other DNA

SEQ ID NO: 27 - DNA, 1091 bp, Fragaria sp.
  source 1..1091: mol_type = other DNA
atggcaagaatgtcggaacctctagctgttggaagagtcataggagatgttcttgattccttcaccccaactacaaaaatgattgtcacttacaacaccaagctcgtctgcaatggacatgagctcttcccttctgcagtcaccgccaaacctagagttgagattcaaggaggcgacatgagatcattcttcactctggtatacatcattttctttctggcctccttttccgccgtttgttttttttatcagatctaccagatcgctaactttagttattttcacaggtaatgacagacccctggccctagtgatccttatttgaaagagcacctgcactggtatatcttacagttaatactgtctatatattatcaaacctagaaacgaagaaacaaagacgataactattcagataattattaataacttcaatgttcaaactagatatatatgttgtcatatactagaaatttgcagtattgaccgaacacattgtccaggttaatttatagaaaagattagttatattaattattgttaatttattctttctcctttacatggtcattcacgtatatattcagaattgtaatcagaaaatctttggatctggaaataaaagctagtaaaaattacatattatgtcacgaactcttagttataatggtggagaatatacatgccactaagcttggatctaacagttttgccttgaaattgaactatacaggattgtgacagacattcctggcaccacagatgctacatttggtaggttaattcacactagttcatgagctaccagtacgttatacagctagtgatgatcagcactgatattgtacgtgtatatactacattaattaacaggaagagaagtggtgagctacgagatgccaaggccaaacataggcatccacaggtttgtgtttgttctcttcatgcaaaaacgaaggcagtcggtgaacccgccttcctcacgggatcacttcaacacccgaaccttcgcagccgaaaacgaccttggtgttcctgttgctgccgtttacttcaatgcacagagagaaacggcagcaagaagacgctag

SEQ ID NO: 28 - DNA, 1074 bp, Fragaria sp.
  source 1..1074: mol_type = other DNA
atggcaagaatgtcggaacctctagctgttggaagagtcataggagatgttcttgattccttcaccccaactacaaaaatgattgtcacttacaacaccaagctcgtctgcaatggacatgagctcttcccttctgcagtcaccgccaaacctagagttgagattcaaggaggcgacatgagatcattcttcactctggtatacatcattttctttctggcctccttttccgccgtttgttttttttatcagatctaccagatcgctaactttagttattttcacaggtaatgacagacccagattgaaagagcacctgcactggtatatcttacagttaatactgtctatatattatcaaacctagaaacgaagaaacaaagacgataactattcagataattattaataacttcaatgttcaaactagatatatatgttgtcatatactagaaatttgcagtattgaccgaacacattgtccaggttaatttatagaaaagattagttatattaattattgttaatttattctttctcctttacatggtcattcacgtatatattcagaattgtaatcagaaaatctttggatctggaaataaaagctagtaaaaattacatattatgtcacgaactcttagttataatggtggagaatatacatgccactaagcttggatctaacagttttgccttgaaattgaactatacaggattgtgacagacattcctggcaccacagatgctacatttggtaggttaattcacactagttcatgagctaccagtacgttatacagctagtgatgatcagcactgatattgtacgtgtatatactacattaattaacaggaagagaagtggtgagctacgagatgccaaggccaaacataggcatccacaggtttgtgtttgttctcttcatgcaaaaacgaaggcagtcggtgaacccgccttcctcacgggatcacttcaacacccgaaccttcgcagccgaaaacgaccttggtgttcctgttgctgccgtttacttcaatgcacagagagaaacggcagcaagaagacgctag

SEQ ID NO: 29 - DNA, 1098 bp, Fragaria sp.; identical to SEQ ID NO: 14
  source 1..1098: mol_type = other DNA

SEQ ID NO: 30 - DNA, 1081 bp, Fragaria sp.
  source 1..1081: mol_type = other DNA
atggcaagaatgtcggaacctctagctgttggaagagtcataggagatgttcttgattccttcaccccaactacaaaaatgattgtcacttacaacaccaagctcgtctgcaatggacatgagctcttcccttctgcagtcaccgccaaacctagagttgagattcaaggaggcgacatgagatcattcttcactctggtatacatcattttctttctggcctccttttccgccgtttgttttttttatcagatctaccagatcgctaactttagttattttcacaggtaatgacagacccagatccttatttgaaagagcacctgcactggtatatcttacagttaatactgtctatatattatcaaacctagaaacgaagaaacaaagacgataactattcagataattattaataacttcaatgttcaaactagatatatatgttgtcatatactagaaatttgcagtattgaccgaacacattgtccaggttaatttatagaaaagattagttatattaattattgttaatttattctttctcctttacatggtcattcacgtatatattcagaattgtaatcagaaaatctttggatctggaaataaaagctagtaaaaattacatattatgtcacgaactcttagttataatggtggagaatatacatgccactaagcttggatctaacagttttgccttgaaattgaactatacaggattgtgacagacattcctggcaccacagatgctacatttggtaggttaattcacactagttcatgagctaccagtacgttatacagctagtgatgatcagcactgatattgtacgtgtatatactacattaattaacaggaagagaagtggtgagctacgagatgccaaggccaaacataggcatccacaggtttgtgtttgttctcttcatgcaaaaacgaaggcagtcggtgaacccgccttcctcacgggatcacttcaacacccgaaccttcgcagccgaaaacgaccttggtgttcctgttgctgccgtttacttcaatgcacagagagaaacggcagcaagaagacgctag

SEQ ID NO: 31 - DNA, 1101 bp, Fragaria sp.
  source 1..1101: mol_type = other DNA
atggcaagaatgtcggaacctctagctgttggaagagtcataggagatgttcttgattccttcaccccaactacaaaaatgattgtcacttacaacaccaagctcgtctgcaatggacatgagctcttcccttctgcagtcaccgccaaacctagagttgagattcaaggaggcgacatgagatcattcttcactctggtatacatcattttctttctggcctccttttccgccgtttgttttttttatcagatctaccagatcgctaactttagttattttcacaggtaatgacagacccagaggtgttcctggccctagtgatccttatttgaaagagcacctgcactggtatatcttacagttaatactgtctatatattatcaaacctagaaacgaagaaacaaagacgataactattcagataattattaataacttcaatgttcaaactagatatatatgttgtcatatactagaaatttgcagtattgaccgaacacattgtccaggttaatttatagaaaagattagttatattaattattgttaatttattctttctcctttacatggtcattcacgtatatattcagaattgtaatcagaaaatctttggatctggaaataaaagctagtaaaaattacatattatgtcacgaactcttagttataatggtggagaatatacatgccactaagcttggatctaacagttttgccttgaaattgaactatacaggattgtgacagacattcctggcaccacagatgctacatttggtaggttaattcacactagttcatgagctaccagtacgttatacagctagtgatgatcagcactgatattgtacgtgtatatactacattaattaacaggaagagaagtggtgagctacgagatgccaaggccaaacataggcatccacaggtttgtgtttgttctcttcatgcaaaaacgaaggcagtcggtgaacccgccttcctcacgggatcacttcaacacccgaaccttcgcagccgaaaacgaccttggtgttcctgttgctgccgtttacttcaatgcacagagagaaacggcagcaagaagacgctag

SEQ ID NO: 32 - DNA, 1010 bp, Fragaria sp.
  source 1..1010: mol_type = other DNA
atggcaagaatgtcggaacctctagctgttggaagagtcataggagatgttcttgattccttcaccccaactacaaaaatgattgtcacttacaacaccaagctcgtctgcaatggacatgagctcttcccttctgcagtcaccgccaaacctagagttgagattcaaggaggcgacatgagatcattcttcactctggtatacatcattttctttctggcctccttttccgccgtttgttttttttatcagatctaccagatcgctaactttagttattttcacaggtaatgacagacccagaaacgaagaaacaaagacgataactattcagataattattaataacttcaatgttcaaactagatatatatgttgtcatatactagaaatttgcagtattgaccgaacacattgtccaggttaatttatagaaaagattagttatattaattattgttaatttattctttctcctttacatggtcattcacgtatatattcagaattgtaatcagaaaatctttggatctggaaataaaagctagtaaaaattacatattatgtcacgaactcttagttataatggtggagaatatacatgccactaagcttggatctaacagttttgccttgaaattgaactatacaggattgtgacagacattcctggcaccacagatgctacatttggtaggttaattcacactagttcatgagctaccagtacgttatacagctagtgatgatcagcactgatattgtacgtgtatatactacattaattaacaggaagagaagtggtgagctacgagatgccaaggccaaacataggcatccacaggtttgtgtttgttctcttcatgcaaaaacgaaggcagtcggtgaacccgccttcctcacgggatcacttcaacacccgaaccttcgcagccgaaaacgaccttggtgttcctgttgctgccgtttacttcaatgcacagagagaaacggcagcaagaagacgctag

SEQ ID NO: 33 - DNA, 1151 bp, Fragaria sp.
  source 1..1151: mol_type = other DNA
atggcaagaatgtcggaacctctagctgttggaagagtcataggagatgttcttgattccttcaccccaactacaaaaatgattgtcacttacaacaccaagctcgtctgcaatggacatgagctcttcccttctgcagtcaccgccaaacctagagttgagattcaaggaggcgacatgagatcattcttcactctggtatacatcattttctttctggcctccttttccgccgtttgttttttttatcagatctaccagatcgctaactttagttattttcacaggtaatgacagacccagatcatggccctagtgagatatactagttattttcacaggtttgttccgccgtttgttcctggccctagtgatccttatttgaaagagcacctgcactggtatatcttacagttaatactgtctatatattatcaaacctagaaacgaagaaacaaagacgataactattcagataattattaataacttcaatgttcaaactagatatatatgttgtcatatactagaaatttgcagtattgaccgaacacattgtccaggttaatttatagaaaagattagttatattaattattgttaatttattctttctcctttacatggtcattcacgtatatattcagaattgtaatcagaaaatctttggatctggaaataaaagctagtaaaaattacatattatgtcacgaactcttagttataatggtggagaatatacatgccactaagcttggatctaacagttttgccttgaaattgaactatacaggattgtgacagacattcctggcaccacagatgctacatttggtaggttaattcacactagttcatgagctaccagtacgttatacagctagtgatgatcagcactgatattgtacgtgtatatactacattaattaacaggaagagaagtggtgagctacgagatgccaaggccaaacataggcatccacaggtttgtgtttgttctcttcatgcaaaaacgaaggcagtcggtgaacccgccttcctcacgggatcacttcaacacccgaaccttcgcagccgaaaacgaccttggtgttcctgttgctgccgtttacttcaatgcacagagagaaacggcagcaagaagacgctag

SEQ ID NO: 34 - DNA, 1095 bp, Fragaria sp.
  source 1..1095: mol_type = other DNA
atggcaagaatgtcggaacctctagctgttggaagagtcataggagatgttcttgattccttcaccccaactacaaaaatgattgtcacttacaacaccaagctcgtctgcaatggacatgagctcttcccttctgcagtcaccgccaaacctagagttgagattcaaggaggcgacatgagatcattcttcactctggtatacatcattttctttctggcctccttttccgccgtttgttttttttatcagatctaccagatcgctaactttagttattttcacaggtaatgacagacccagacctggccctagtgatccttatttgaaagagcacctgcactggtatatcttacagttaatactgtctatatattatcaaacctagaaacgaagaaacaaagacgataactattcagataattattaataacttcaatgttcaaactagatatatatgttgtcatatactagaaatttgcagtattgaccgaacacattgtccaggttaatttatagaaaagattagttatattaattattgttaatttattctttctcctttacatggtcattcacgtatatattcagaattgtaatcagaaaatctttggatctggaaataaaagctagtaaaaattacatattatgtcacgaactcttagttataatggtggagaatatacatgccactaagcttggatctaacagttttgccttgaaattgaactatacaggattgtgacagacattcctggcaccacagatgctacatttggtaggttaattcacactagttcatgagctaccagtacgttatacagctagtgatgatcagcactgatattgtacgtgtatatactacattaattaacaggaagagaagtggtgagctacgagatgccaaggccaaacataggcatccacaggtttgtgtttgttctcttcatgcaaaaacgaaggcagtcggtgaacccgccttcctcacgggatcacttcaacacccgaaccttcgcagccgaaaacgaccttggtgttcctgttgctgccgtttacttcaatgcacagagagaaacggcagcaagaagacgctag

SEQ ID NO: 35 - DNA, 1110 bp, Fragaria sp.
  source 1..1110: mol_type = other DNA
atggcaagaatgtcggaacctctagctgttggaagagtcataggagatgttcttgattccttcaccccaactacaaaaatgattgtcacttacaacaccaagctcgtctgcaatggacatgagctcttcccttctgcagtcaccgccaaacctagagttgagattcaaggaggcgacatgagatcattcttcactctggtatacatcattttctttctggcctccttttccgccgtttgttttttttatcagatctaccagatcgctaactttagttattttcacaggtaatgacagacccagatcgctaactttagttcctggccctagtgatccttatttgaaagagcacctgcactggtatatcttacagttaatactgtctatatattatcaaacctagaaacgaagaaacaaagacgataactattcagataattattaataacttcaatgttcaaactagatatatatgttgtcatatactagaaatttgcagtattgaccgaacacattgtccaggttaatttatagaaaagattagttatattaattattgttaatttattctttctcctttacatggtcattcacgtatatattcagaattgtaatcagaaaatctttggatctggaaataaaagctagtaaaaattacatattatgtcacgaactcttagttataatggtggagaatatacatgccactaagcttggatctaacagttttgccttgaaattgaactatacaggattgtgacagacattcctggcaccacagatgctacatttggtaggttaattcacactagttcatgagctaccagtacgttatacagctagtgatgatcagcactgatattgtacgtgtatatactacattaattaacaggaagagaagtggtgagctacgagatgccaaggccaaacataggcatccacaggtttgtgtttgttctcttcatgcaaaaacgaaggcagtcggtgaacccgccttcctcacgggatcacttcaacacccgaaccttcgcagccgaaaacgaccttggtgttcctgttgctgccgtttacttcaatgcacagagagaaacggcagcaagaagacgctag

SEQ ID NO: 36 - DNA, 1085 bp, Fragaria sp.
  source 1..1085: mol_type = other DNA
atggcaagaatgtcggaacctctagctgttggaagagtcataggagatgttcttgattccttcaccccaactacaaaaatgattgtcacttacaacaccaagctcgtctgcaatggacatgagctcttcccttctgcagtcaccgccaaacctagagttgagattcaaggaggcgacatgagatcattcttcactctggtatacatcattttctttctggcctccttttccgccgtttgttttttttatcagatctaccagatcgctaactttagttattttcacaggtaatgacagacccatagtgatccttatttgaaagagcacctgcactggtatatcttacagttaatactgtctatatattatcaaacctagaaacgaagaaacaaagacgataactattcagataattattaataacttcaatgttcaaactagatatatatgttgtcatatactagaaatttgcagtattgaccgaacacattgtccaggttaatttatagaaaagattagttatattaattattgttaatttattctttctcctttacatggtcattcacgtatatattcagaattgtaatcagaaaatctttggatctggaaataaaagctagtaaaaattacatattatgtcacgaactcttagttataatggtggagaatatacatgccactaagcttggatctaacagttttgccttgaaattgaactatacaggattgtgacagacattcctggcaccacagatgctacatttggtaggttaattcacactagttcatgagctaccagtacgttatacagctagtgatgatcagcactgatattgtacgtgtatatactacattaattaacaggaagagaagtggtgagctacgagatgccaaggccaaacataggcatccacaggtttgtgtttgttctcttcatgcaaaaacgaaggcagtcggtgaacccgccttcctcacgggatcacttcaacacccgaaccttcgcagccgaaaacgaccttggtgttcctgttgctgccgtttacttcaatgcacagagagaaacggcagcaagaagacgctag

SEQ ID NO: 37 - DNA, 1095 bp, Fragaria sp.; identical to SEQ ID NO: 34
  source 1..1095: mol_type = other DNA

SEQ ID NO: 38 - DNA, 1102 bp, Fragaria sp.
  source 1..1102: mol_type = other DNA
atggcaagaatgtcggaacctctagctgttggaagagtcataggagatgttcttgattccttcaccccaactacaaaaatgattgtcacttacaacaccaagctcgtctgcaatggacatgagctcttcccttctgcagtcaccgccaaacctagagttgagattcaaggaggcgacatgagatcattcttcactctggtatacatcattttctttctggcctccttttccgccgtttgtttttttttatcagatctaccagatcgctaactttagttattttcacaggtaatgacagagcacctgcactggtatatcttagtgatccttatttgaaagagcacctgcactggtatatcttacagttaatactgtctatatattatcaaacctagaaacgaagaaacaaagacgataactattcagataattattaataacttcaatgttcaaactagatatatatgttgtcatatactagaaatttgcagtattgaccgaacacattgtccaggttaatttatagaaaagattagttatattaattattgttaatttattctttctcctttacatggtcattcacgtatatattcagaattgtaatcagaaaatctttggatctggaaataaaagctagtaaaaattacatattatgtcacgaactcttagttataatggtggagaatatacatgccactaagcttggatctaacagttttgccttgaaattgaactatacaggattgtgacagacattcctggcaccacagatgctacatttggtaggttaattcacactagttcatgagctaccagtacgttatacagctagtgatgatcagcactgatattgtacgtgtatatactacattaattaacaggaagagaagtggtgagctacgagatgccaaggccaaacataggcatccacaggtttgtgtttgttctcttcatgcaaaaacgaaggcagtcggtgaacccgccttcctcacgggatcacttcaacacccgaaccttcgcagccgaaaacgaccttggtgttcctgttgctgccgtttacttcaatgcacagagagaaacggcagcaagaagacgctag

SEQ ID NO: 39 - DNA, 1095 bp, Fragaria sp.
  source 1..1095: mol_type = other DNA
atggcaagaatgtcggaacctctagctgttggaagagtcataggagatgttcttgattccttcaccccaactacaaaaatgattgtcacttacaacaccaagctcgtctgcaatggacatgagctcttcccttctgcagtcaccgccaaacctagagttgagattcaaggaggcgacatgagatcattcttcactctggtatacatcattttctttctggcctccttttccgccgtttgtttttttttatcagatctaccagatcgctaactttagttattttcacaggtaatgacagacccaggctggccctagtgatccttatttgaaagagcacctgcactggtatatcttacagttaatactgtctatatattatcaaacctagaaacgaagaaacaaagacgataactattcagataattattaataacttcaatgttcaaactagatatatatgttgtcatatactagaaatttgcagtattgaccgaacacattgtccaggttaatttatagaaaagattagttatattaattattgttaatttattctttctcctttacatggtcattcacgtatatattcagaattgtaatcagaaaatctttggatctggaaataaaagctagtaaaaattacatattatgtcacgaactcttagttataatggtggagaatatacatgccactaagcttggatctaacagttttgccttgaaattgaactatacaggattgtgacagacattcctggcaccacagatgctacatttggtaggttaattcacactagttcatgagctaccagtacgttatacagctagtgatgatcagcactgatattgtacgtgtatatactacattaattaacaggaagagaagtggtgagctacgagatgccaaggccaaacataggcatccacaggtttgtgtttgttctcttcatgcaaaaacgaaggcagtcggtgaacccgccttcctcacgggatcacttcaacacccgaaccttcgcagccgaaaacgaccttggtgttcctgttgctgccgtttacttcaatgcacagagagaaacggcagcaagaagacgctag

SEQ ID NO: 40 - DNA, 1095 bp, Fragaria sp.; identical to SEQ ID NO: 25
  source 1..1095: mol_type = other DNA

SEQ ID NO: 41 - DNA, 1096 bp, Fragaria sp.; identical to SEQ ID NO: 8
  source 1..1096: mol_type = other DNA

SEQ ID NO: 42 - protein, 169 aa, Fragaria sp.
MARMSEPLAVGRVIGDVLDSFTPTTKMIVTYNTKLVCNGHELFPSAVTAKPRVEIQGGDMRSFFTLVMTDPGPSDPYLKEHLHWIVTDIPGTTDATFGREVVSYEMPRPNIGIHRFVFVLFMQKRRQSVNPPSSRDHFNTRTFAAENDLGVPVAAVYFNAQRETAARRR

SEQ ID NO: 43 - protein, 76 aa, Fragaria sp.
MARMSEPLAVGRVIGDVLDSFTPTTKMIVTYNTKLVCNGHELFPSAVTAKPRVEIQGGDMRSFFTLVMTDPDCSWP

SEQ ID NO: 44 - protein, 171 aa, Fragaria sp.
MARMSEPLAVGRVIGDVLDSFTPTTKMIVTYNTKLVCNGHELFPSAVTAKPRVEIQGGDMRSFFTLVMTDPDPGPSDPYLKEHLHWIVTDIPGTTDATFGREVVSYEMPRPNIGIHRFVFVLFMQKRRQSVNPPSSRDHFNTRTFAAENDLGVPVAAVYFNAQRETAARRR

SEQ ID NO: 45 - protein, 76 aa, Fragaria sp.; identical to SEQ ID NO: 43

SEQ ID NO: 46 - protein, 74 aa, Fragaria sp.
MARMSEPLAVGRVIGDVLDSFTPTTKMIVTYNTKLVCNGHELFPSAVTAKPRVEIQGGDMRSFFTLVKSTCTGL

SEQ ID NO: 47 - protein, 171 aa, Fragaria sp.
MARMSEPLAVGRVIGDVLDSFTPTTKMIVTYNTKLVCNGHELFPSAVTAKPRVEIQGGDMRSFFTLVMTDPEPGPSDPYLKEHLHWIVTDIPGTTDATFGREVVSYEMPRPNIGIHRFVFVLFMQKRRQSVNPPSSRDHFNTRTFAAENDLGVPVAAVYFNAQRETAARRR

SEQ ID NO: 48 - protein, 171 aa, Fragaria sp.
MARMSEPLAVGRVIGDVLDSFTPTTKMIVTYNTKLVCNGHELFPSAVTAKPRVEIQGGDMRSFFTLVMTDPVPGPSDPYLKEHLHWIVTDIPGTTDATFGREVVSYEMPRPNIGIHRFVFVLFMQKRRQSVNPPSSRDHFNTRTFAAENDLGVPVAAVYFNAQRETAARRR

SEQ ID NO: 49 - protein, 79 aa, Fragaria sp.
MARMSEPLAVGRVIGDVLDSFTPTTKMIVTYNTKLVCNGHELFPSAVTAKPRVEIQGGDMRSFFTLVMTDPDLALVILI

SEQ ID NO: 50 - protein, 171 aa, Fragaria sp.; identical to SEQ ID NO: 48

SEQ ID NO: 51 - protein, 75 aa, Fragaria sp.
MARMSEPLAVGRVIGDVLDSFTPTTKMIVTYNTKLVCNGHELFPSAVTAKPRVEIQGGDMRSFFTLVMTDPGSWP

SEQ ID NO: 52 - protein, 171 aa, Fragaria sp.; identical to SEQ ID NO: 48

SEQ ID NO: 53 - protein, 75 aa, Fragaria sp.
MARMSEPLAVGRVIGDVLDSFTPTTKMIVTYNTKLVCNGHELFPSAVTAKPRVEIQGGDMRSFFTLVMTDPDSWP

SEQ ID NO: 54 - protein, 171 aa, Fragaria sp.; identical to SEQ ID NO: 44

SEQ ID NO: 55 - protein, 80 aa, Fragaria sp.
MARMSEPLAVGRVIGDVLDSFTPTTKMIVTYNTKLVCNGHELFPSAVTAKPRVEIQGGDMRSFFTLVMTDPEFLALVILI

SEQ ID NO: 56 - protein, 169 aa, Fragaria sp.; identical to SEQ ID NO: 42

SEQ ID NO: 57 - protein, 79 aa, Fragaria sp.; identical to SEQ ID NO: 49

SEQ ID NO: 58 - protein, 171 aa, Fragaria sp.; identical to SEQ ID NO: 44

SEQ ID NO: 59 - protein, 79 aa, Fragaria sp.
MARMSEPLAVGRVIGDVLDSFTPTTKMIVTYNTKLVCNGHELFPSAVTAKPRVEIQGGDMRSFFTLVMTDPVLALVILI

SEQ ID NO: 60 - protein, 168 aa, Fragaria sp.
MARMSEPLAVGRVIGDVLDSFTPTTKMIVTYNTKLVCNGHELFPSAVTAKPRVEIQGGDMRSFFTLVMTPGPSDPYLKEHLHWIVTDIPGTTDATFGREVVSYEMPRPNIGIHRFVFVLFMQKRRQSVNPPSSRDHFNTRTFAAENDLGVPVAAVYFNAQRETAARRR

SEQ ID NO: 61 - protein, 171 aa, Fragaria sp.; identical to SEQ ID NO: 44

SEQ ID NO: 62 - protein, 172 aa, Fragaria sp.
MARMSEPLAVGRVIGDVLDSFTPTTKMIVTYNTKLVCNGHELFPSAVTAKPRVEIQGGDMRSFFTLVMTDPDVPGPSDPYLKEHLHWIVTDIPGTTDATFGREVVSYEMPRPNIGIHRFVFVLFMQKRRQSVNPPSSRDHFNTRTFAAENDLGVPVAAVYFNAQRETAARRR

SEQ ID NO: 63 - protein, 80 aa, Fragaria sp.; identical to SEQ ID NO: 55

SEQ ID NO: 64 - protein, 75 aa, Fragaria sp.
MARMSEPLAVGRVIGDVLDSFTPTTKMIVTYNTKLVCNGHELFPSAVTAKPRVEIQGGDMRSFFTLVMTDPDPWP

SEQ ID NO: 65 - protein, 77 aa, Fragaria sp.
MARMSEPLAVGRVIGDVLDSFTPTTKMIVTYNTKLVCNGHELFPSAVTAKPRVEIQGGDMRSFFTLVMTDPALVILI

SEQ ID NO: 66 - protein, 74 aa, Fragaria sp.
MARMSEPLAVGRVIGDVLDSFTPTTKMIVTYNTKLVCNGHELFPSAVTAKPRVEIQGGDMRSFFTLVMTDPAWP

SEQ ID NO: 67 - protein, 80 aa, Fragaria sp.; identical to SEQ ID NO: 55

SEQ ID NO: 68 - protein, 73 aa, Fragaria sp.
MARMSEPLAVGRVIGDVLDSFTPTTKMIVTYNTKLVCNGHELFPSAVTAKPRVEIQGGDMRSFFTLVMTDPWP

SEQ ID NO: 69 - protein, 72 aa, Fragaria sp.
MARMSEPLAVGRVIGDVLDSFTPTTKMIVTYNTKLVCNGHELFPSAVTAKPRVEIQGGDMRSFFTLVMTDPD

SEQ ID NO: 70 - protein, 80 aa, Fragaria sp.; identical to SEQ ID NO: 55

SEQ ID NO: 71 - protein, 166 aa, Fragaria sp.
MARMSEPLAVGRVIGDVLDSFTPTTKMIVTYNTKLVCNGHELFPSAVTAKPRVEIQGGDMRSFFTLVMTDPDPYLKEHLHWIVTDIPGTTDATFGREVVSYEMPRPNIGIHRFVFVLFMQKRRQSVNPPSSRDHFNTRTFAAENDLGVPVAAVYFNAQRETAARRR

SEQ ID NO: 72 - protein, 81 aa, Fragaria sp.
MARMSEPLAVGRVIGDVLDSFTPTTKMIVTYNTKLVCNGHELFPSAVTAKPRVEIQGGDMRSFFTLVMTDPEVFLALVILI

SEQ ID NO: 73 - protein, 156 aa, Fragaria sp.
MARMSEPLAVGRVIGDVLDSFTPTTKMIVTYNTKLVCNGHELFPSAVTAKPRVEIQGGDMRSFFTLVMTDPIVTDIPGTTDATFGREVVSYEMPRPNIGIHRFVFVLFMQKRRQSVNPPSSRDHFNTRTFAAENDLGVPVAAVYFNAQRETAARRR

SEQ ID NO: 74 - protein, 79 aa, Fragaria sp.
MARMSEPLAVGRVIGDVLDSFTPTTKMIVTYNTKLVCNGHELFPSAVTAKPRVEIQGGDMRSFFTLVMTDPDHGPSEIY

SEQ ID NO: 75 - protein, 79 aa, Fragaria sp.; identical to SEQ ID NO: 49

SEQ ID NO: 76 - protein, 73 aa, Fragaria sp.
MARMSEPLAVGRVIGDVLDSFTPTTKMIVTYNTKLVCNGHELFPSAVTAKPRVEIQGGDMRSFFTLVMTDPDR

SEQ ID NO: 77 - protein, 71 aa, Fragaria sp.
MARMSEPLAVGRVIGDVLDSFTPTTKMIVTYNTKLVCNGHELFPSAVTAKPRVEIQGGDMRSFFTLVMTDP

SEQ ID NO: 78 - protein, 79 aa, Fragaria sp.; identical to SEQ ID NO: 49

SEQ ID NO: 79 - protein, 81 aa, Fragaria sp.
MARMSEPLAVGRVIGDVLDSFTPTTKMIVTYNTKLVCNGHELFPSAVTAKPRVEIQGGDMRSFFTLVMTEHLHWYILVILI

SEQ ID NO: 80 - protein, 74 aa, Fragaria sp.
MARMSEPLAVGRVIGDVLDSFTPTTKMIVTYNTKLVCNGHELFPSAVTAKPRVEIQGGDMRSFFTLVMTDPGWP

SEQ ID NO: 81 - protein, 74 aa, Fragaria sp.; identical to SEQ ID NO: 66

SEQ ID NO: 82 - protein, 79 aa, Fragaria sp.; identical to SEQ ID NO: 49

SEQ ID NO: 83 - protein, 159 aa, Fragaria X ananassa
LAVGRVIGDVLDSFTPTTKMIVTYNTKLVCNGHELFPSAVTAKPKVEIQGGDMRSFFTLVMTDPDVPGPSDPYLKEHLHWIVTDIPGTTDATFGREVVSYEMPRPNIGIHRFVFVLFMQKRRQSVNPPSSRDHFNTRTFAAENDLGIPVAAVYFNAQRE

SEQ ID NO: 84 - protein, 159 aa, Fragaria X ananassa
LAVGRVIGDVLDSFTPTTKMIVTYNTKLVCNGHELFPSAVTAKPRVEIQGGDMRSFFTLVMTDPDVPGPSDPYLKEHLHWIVTDIPGTTDATFGREVVSYEMPRPNIGIHRFVFVLFMQKRRQSVNPPSSRDHFNTRTFAAENDLGVPVAAVYFNAQRE

SEQ ID NO: 85 - protein, 159 aa, Fragaria X ananassa
LAVGRVIGDVLDSFTPTTKMIVSYNSKLVCNGHELFPSAVTAKPRVEIQGGDMRSFFTLVMTDPDVPGPSDPYLKEHLHWIVTDIPGTTDATFGREVVSYEMPRPNIGIHRFVFVLFMQKRRQSVNPPSSRDHFNTRTFAAENDLGVPVAAVYFNAQRE

SEQ ID NO: 86 - protein, 173 aa, Fragaria vesca
MAKISDPLVVGRVIGDVIDCFTPSVKMTVTYNSSKKVYNGHELYPSSVTVKPKVEVHGGDMRSFFTLVMTDPDVPGPSDPYLKEHLHWIVTDIPGTTDNTFGKEVVKYEMPRPNIGIHRFVFLLFKQKGRQTVIPPPSKDHFDSRKFAETNEFGLPVAAVFFNAQRETAARRR

SEQ ID NO: 87 - protein, 173 aa, Fragaria X ananassa
MAKISDPLVVGRVIGDVIDCFTPSVKMTVTYNSSKKVYNGHELYPSSVTVKPKVEVHGGDMRSFFTLVMTDPDVPGPSDPYLKEHLHWIVTDIPGTTDNTFGREVVKYEMPRPNIGIHRFVFLLFKQKGRQTVIPPPSKDHFNSRKFAEANEFGLPVAAVFFNAQRETAARRR

SEQ ID NO: 88 - protein, 173 aa, Fragaria X ananassa
MAKISDPLVVGRVIGDVIDCFTPSVKMTVTYNSSKKVYNGHELYPSSVTVKPKVEVHGGDMRSFFTLVMTDPDVPGPSDPYLKEHLHWIVTDIPGTTDNTFGREVVKYEMPRPNIGIHRFVFLLFKQKGRQTVIPPPSKDHFNSRKFAEANEFGLPVAAVYFNSQRETAARRR

SEQ ID NO: 89 - protein, 173 aa, Fragaria X ananassa
MAKISDPLVVGRVIGDVIDCFTPSVKMTVTYNSSKKVYNGHELYPSSVTVKPKVEVHGGDMRSFFTLVMTDPDVPGPSDPYLKEHLHWIVTDIPGTTDNTFGREVVTYEMPRPNIGIHRFVFLLFKQKGRQTVIPPPSKDHFDSRKFAETNEFGLPVAAVFFNAQRETAARRR

SEQ ID NO: 90 - protein, 171 aa, Fragaria vesca
METLTLGRVVGEVVDMFTPSVKMNVVYSSCNKQVSNGHEIMPSVIAAKPRVDIGGEDMRAAYTLIMTDPDYPSPSDPCLREHLHWMVTDIPGTTDVSFGREVVEYETPKPVVGIHRYVFLLFQQTRGRQTVRVPASRDNFNTRKFSQENSLGLPVAAVYFNAQRETAARRR

SEQ ID NO: 91 - protein, 171 aa, Fragaria X ananassa
METLTLGRVVGEVVDMFTPSVKMNVVYSSCNKQVSNGHEIMPSVIAAKPRVDIGGEDMRAAYTLIMTDPDYPSPSDPYLREHLHWMVTDIPGTTDVSFGREVLEYETPKPVVGIHRYVFLLFQQTRGRQTVRVPASRDNFNTRKFSQENSLGLPVAAVYFNAQRETAARRR

SEQ ID NO: 92 - protein, 171 aa, Fragaria X ananassa
METLILGRVVGEVVDMFTPSVKMNVVYSSCNKQVSNGHEIMPSVIAAKPRVDIGGEDMRAAYTLIMTDPDYPGPSDPYLREHLHWMVTDIPGTTDVSFGREVLEYETPKPVVGIHRYVFLLFQQTRGRQTVRVPASRDNFNTRKFSQENSLGLPVAAVYFNAQRETAARRR

SEQ ID NO: 93 - protein, 171 aa, Fragaria X ananassa; identical to SEQ ID NO: 90

SEQ ID NO: 94 - protein, 171 aa, Fragaria X ananassa
METLILGRVVGEVVDMFTPSVKMNVVYSSCNKQVSNGHEIMPSVIAAKPRVEIGGEDMRAAYTLIMTDPDYPGPSDPYLREHLHWMVTDIPGTTDVSFGREVLEYETPKPVVGIHRYVFLLFQQTRGRQTVRVPASRDNFNTRKFSQENSLGLPVAAVYFNAQRETAARRR

SEQ ID NO: 95 - protein, 103 aa, Fragaria X ananassa
METLTLGRVVGEVVDMFTPSVKMNVVYSSCNKQVSNGHEIMPSVIAAKPRVDIGGEDMRAAYTLIMTDPDYPSPSDPYLREHLHWMVTDIPGTTDVSFGREVL

SEQ ID NO: 96 - protein, 177 aa, Fragaria X ananassa
MARARDQEPLVVGRVIGDVIEPFTKSVSLRMTYSNNREVTSGCELKPSHVINRPRVQIGGDDLRNFYTLVMVDPDAPSPSDPNLKEYLHWLVTDIPATTGASFGQEIVSYESPRPSIGIHRFVSVLFRQLGRQTVYAPGWRQNFNTREFAENYNLGSPVAAVFFNCQRESGSGGRRM

SEQ ID NO: 97 - protein, 177 aa, Fragaria X ananassa
MARARDQEPLVVGRVIGDVIEPFTKSVSLRMTYSNNREVTSGCELKPSHVINRPRVQIGGDDLRIFYTLVMVDPDAPSPSDPNLKEYLHWLVTDIPATTGASFGQEILSYESPRPSMGIHRFVLVLFHQLGRQTVYAPGWRQNFNTREFAENCNLGSPVAAVFCNCQRESGSGGRRM

SEQ ID NO: 98 - protein, 177 aa, Fragaria X ananassa
MARARDQEPLVVGRVIGDVIEPFTKSVSLRMTYSNNREVTSGCELKPSHVINRPRVQIGGDDLRNFYTLVMVDPDAPSPSDPNLKEYLHWLVTDIPATTGASFGQEIVSYESPRPSMGIHRFVSVLFRQLGRQTVYAPGWRQNFNTREFAENYNLGSPVAAVYFNCQRESGSGGRRM

SEQ ID NO: 99 - protein, 177 aa, Fragaria X ananassa
MARARDQEPLVIGRVIGDVIEPFTKSVSLRMTYSNNREVTSGCELKPSHVINRPRVQIGGDDLRIFYTLVMVDPDAPSPSDPNLKEYLHWLVTDIPATTGASFGQEILSYESPRPSMGIHRFVLVLFHQLGRQTVYAPGWRQNFNTREFAENCNLGSPVAAVFCNCQRESGSGGRRM

SEQ ID NO: 100 - protein, 176 aa, Fragaria X ananassa
MPRDRDPLVVGRVIGDVLDPFTKSVSLRVTYTSKEVNNGCELKPSQVVRQPRVDIGGEDLRTFYTLVMVDPDAPSPSDPNLKEYLHWLVTDIPATAGAVFGQETVCYESPRPTAGIHRFLFVLFRQLGRQTVYAPGWRQNFNTRDFAELYNLGSPVAAVYFNCQRESGSGGRRRSS

SEQ ID NO: 101 - protein, 176 aa, Fragaria X ananassa
MPRDRDPLVVGRVIGDVLDPFTKSVSLRVTYTSKEVNNGCELKPSQVVSQPRVDIGGEDLRTFYTLVMVDPDAPSPSDPNLKEYLHWLVTDIPATAGAVFGQEIVCYESPRPTAGIHRFLFVLFRQLGRQTVYAPGWRQNFNTRDFAELYNLGSPAAAVYFNCQRESGSGGRRRSS

SEQ ID NO: 102 - protein, 176 aa, Fragaria X ananassa
MPRDRDPLVVGRVIGDVLDPFTKSVSLRVTYTSKEVNNGCELKPSQVVSQPRVDIGGEDLRTFYTLVMVDPDAPSPSDPNLKEYLHWLVTDIPATAGAVFGQEIVCYESPRPTAGIHRFLFVLFRQLGRQTVYAPGWRQNFNTRDFAELYNLGSPVAAVYFNCQRESGSGGRRRSS

SEQ ID NO: 103 - protein, 596 aa, Fragaria vesca
MALRVSLVAAALLLLSAASTTTTATAAAAQALPGCSDKCGNLTITYPFGMEEGCYLREEFFINCVRTTAYLTTGDHIVDISVDDGELRMLNDPEQNCYDAQGNLMSSFHRSLFTLPFPYTISDTKNKFYAVGCDTSAMLQGFRGEEELSTGCISICIRNSTGSINQDYSCSGVGCCQTNIPYGMNNQTVTLDKYENNATFVQFYPCSYAFIAEQGHFNSSLACLIELFNFKAFPMVVNWAIGNDTDSCDEAQDRYDFACKANSICINRPRGYLCQCLPGYEGNPYHPDGCQDIDECKAANPCLHGRCSNIPGKYSCSCHKGYKHDPINDKICIKDDSKRQLEINLLHIFLEEGAVGTHEISTSEDCWDVVSTSSGPFTDGGTTAADASKYGVKQIDSPKVPRVALQPLEQRHLILKSMAKARDQDALVVSRVIGDIIEPFTKSVSLRMTYINNREFTNGSELKPSHVVHRPRVDIGGDDLRNFYTLIMVDPDAPNPSEPNLKEYLHWLVTDIPATTGASFGQEIVSYESPRPAMGIHRFVSVLYRQLGRKTVYAPEWRQNFNTRKFAENYNLGSPVAAVYFNCQRETGCGGRRTIM

SEQ ID NO: 104 - protein, 595 aa, Fragaria X ananassa
MALRVSLVAAALLLLSAASTTTTATAAAAQALPGYSDKCGNLTITYPFGMEEGCYLREEFFINCVRTTAYLTTGDHIVDISVDDGELRMLNDPEQNCYDAQGNLMSSFHRSLFTLPFPYTISDTKNKFYAVGCDTSAMLQGFRGEEELSTGCISICIRNSTGSINQDYSCSGVGCCQTNIPYGMNNQTLTLDKYENNATFVQFYPCSYAFIAEQGHFNSSLACLIELFNFKAFPMVVNWAIGNDTDSCDEAQDRYDFACKANSICINRPRGYLCQCLPGYEGNPYHPDGCQDIDECKAANPCLHGRCSNIPGKYSCSCHKGYKHDPINDKICIKDDSKRQLEINLLHIFLEEGTVGTHEISTEDCWDVVSTSSGPFTDGGTAAADASKYGVKQIDSPKVPRVALQPLEQRHLILKSMAKARDQDALVVSRVIGDVIEPFTKSVSLRMTYINNREFANGSELKPSHVVHRPRVDIGGDDLRNFYTLIMVDPDAPNPSEPNLKEYLHWLVTDIPATTGASFGQEIVSYESPRPAMGIHRFVSVLYRQLGRKTVYAPEWRQNFNTRKFAENYNLGSPVAAVYFNCQRETGCGGRRTIM

SEQ ID NO: 105 - protein, 324 aa, Fragaria vesca
MRCRPLISQPGAPELLSSRVFSYEDGGRVLVQMDLHTLKTLRDGSCVFLQGSQSVTQVLPLECFMVLIAKKLWGENDSHIGSFVDRCLAVTDGCDRAIWMEETEGWWFYLIDGGPRVESFACLLSCFLLFLFFFFFFFFFFFFSLFSFFPITMAVSVDPLVVGRVIGDVVDMFVPTVNMSVYFGSKHVTNGCDIKPSIAVSPPKVTFSGHPGELYTLVMTDPDAPSPSEPSMREWVHWILADIPGGTNPIRGKEILPYVGPRPPVGIHRYILVLFQQKAPMGLVEQPPTRAHFNTRYFAAQLDLGLPVSTVYFNAQKEPANRRR

SEQ ID NO: 106 - protein, 172 aa, Fragaria X ananassa; identical to SEQ ID NO: 62

SEQ ID NO: 107 - protein, 172 aa, Rubus idaeus
MARMSEPLVVGRVIGDVLDSFTPTTKMIVTYTTRHVSNGYELLPSAVTTKPRVEIQGGDMRSFFTLVMTDPDAPGPSDPYLKEHLHWVVTDIPGTTDATFGREVVSYEMPRPNIGIHRFVFVLFRQKRRQSVNPPSSRDHFNTRSFAAENDLGLPVAAVYFNAQRETAARRR

SEQ ID NO: 108 - protein, 172 aa, Rubus occidentalis; identical to SEQ ID NO: 107

SEQ ID NO: 109 - protein, 172 aa, Rubus argutus; identical to SEQ ID NO: 107

SEQ ID NO: 110 - protein, 172 aa, Malus domestica
MARVPEPLVVGRVIGDVLDSFTPTTHMSVTYNAKLVCNGLELFPSVVTAKPRVEIQGGELRSFFTLVMTDPDCPGPSDPYLREHLHWIVTDIPGTTDAAFGREALSYEMPRPNIGIHRFVFVLFKQKRRQSINIPSSRDCFSTRSFAAENDLGLPVAAVYFNAQRESAARRR

SEQ ID NO: 111 - protein, 172 aa, Malus domestica
MKRASEPLVVGRVIGDVLDSFTATTKMSVTYNTKLVCNGLELFPSVVTAKPRVEIQGGDMRSFFTLVMTDPDFPGPSDPYLREHLHWIVTDIPGTTDATFGREVVSYEMPKPNIGIHRFVFVLFKQNQRQSINTPSSRDHFSTRSFAAENDLGLPVAAVYFNAQRETAARRR

SEQ ID NO: 112 - protein, 173 aa, Vaccinium corymbosum
MARMSAEPLIVGRVIGDVLDPFTPTVKMLATYNNKQVSNGHEFYPSQVTTKPRVEIHGGDMRTFFTLVMTDPDVPGPSDPYLREHLHWIVTDIPGTTDATFGREVVSYEIPRPNIGIHRFVFVLFKQKSRQAISPPSSRDHFTTRCFAEENDLGLPVAAVFFNAQRETAARRR

SEQ ID NO: 113 - protein, 173 aa, Vaccinium corymbosum; identical to SEQ ID NO: 112

SEQ ID NO: 114 - protein, 173 aa, Vaccinium corymbosum
MARMSAEPLIVGRVIGDVLDPFTPTVKMLATYNNRQVSNGHEFYPSQVTTKPRVEIHGGDMRTFFTLVMTDPDVPGPSDPYLREHLHWIVTDIPGTTDATFGREVVSYEIPRPNIGIHRFVFVLFKQKSRQAISPPSSRDHFTTRCFAEENDLGLPVAAVFFNAQRETAARRR

SEQ ID NO: 115 - protein, 173 aa, Vaccinium darrowii
MARMSAEPLIVGRVIGDVLDPFTPTVKMLATYNNRQVSNGHEFYPSQVTTKPRVEIHGGDMRTFFTLVMTDPDVPGPSDPYLREHLHWIVTDIPGTTDATFGREVVTYEIPRPNIGIHRFVFVLFKQKSRQAISPPSSRDHFTTRCFAEENDLGLPVAAVFFNAQRETAARRR

SEQ ID NO: 116 - protein, 146 aa, Vaccinium corymbosum
MLATYNNRQVSNGHEFYPSQVTTKPRVEIHGGDMRTFFTLVMTDPDVPGPSDPYLREHLHWIVTDIPGTTDATFGREVVSYEIPRPNIGIHRFVFVLFKQKSRQAISPPSSRDHFTTRCFAEENDLGLPVAAVFFNAQRETAARRR

SEQ ID NO: 117 - protein, 177 aa, Fragaria X ananassa; identical to SEQ ID NO: 96